(19) United States
(12) Patent Application Publication
(10) Pub. No.: US 2026/0108689 A1
(43) Pub. Date: Apr. 23, 2026

(54) SYSTEMS AND METHODS FOR SENSOR KITS

(71) Applicant: RESMED PTY LTD, Bella Vista, New South Wales (AU)

(72) Inventors: Michael James DENT, Sydney (AU); Andrew William GILLETT, Sydney (AU); Dmitri Anatolievich DOUDKINE, Sydney (AU); David CREUSOT, Sydney (AU); Andrew CHAN, Sydney (AU); Ting Lee TEH, Sydney (AU); Evan Latimer HOLLINS, Sydney (AU)

(21) Appl. No.: 19/112,884

(22) PCT Filed: Sep. 18, 2023
(86) PCT No.: PCT/IB2023/059247

§ 371 Date: Mar. 18, 2025

Related U.S. Application Data
(60) Provisional application No. 63/376,296, filed on Sep. 20, 2022.
(60) Provisional application No. 63/376,340, filed on Sep. 20, 2022.

(51) Int. Cl. A61M 16/00; A61M 16/06; A61M 16/08; A61M 16/10; A61M 16/16

(52) U.S. Cl. CPC A61M 16/0003; A61M 16/024; A61M 16/0672; A61M 16/0816; A61M 16/1075; A61M 16/161; A61M 2016/0027; A61M 2202/0208; A61M 2205/3334; A61M 2205/3368; A61M 2205/502; A61M 2209/086

(57) ABSTRACT

A system for sensing parameters associated with a respiratory therapy (“RPT”) system may comprise a circuit board; and at least one sensor mounted on the circuit board. The circuit board may be configured to be coupled to a patient interface of the RPT system, such that the at least one sensor is configured to sense a parameter within a plenum chamber of the patient interface and a parameter of an atmosphere outside of the plenum chamber.

### 1 CROSS-REFERENCE TO RELATED APPLICATIONS

This application claims the benefit of priority from U.S. Provisional Application Nos. 63/376,296 and 63/376,340, filed on Sep. 20, 2022, each of which is incorporated by reference herein in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 2.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

Air pressure generators are known in a range of applications, e.g. industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, pertaining to one or more of: comfort, noise, ease of use, efficacy, size, weight, manufacturability, cost, and reliability.

An example of the special requirements of certain RPT devices is acoustic noise.

| | A-weighted | |
|---|---|---|
| | sound pressure | Year |
| RPT Device name | level dB(A) | (approx.) |
| C-Series TangoTM | 31.9 | 2007 |
| C-Series TangoTM with Humidifier | 33.1 | 2007 |
| S8 EscapeTM II | 30.5 | 2005 |
| S8 EscapeTM II with H4iTM Humidifier | 31.1 | 2005 |
| S9 AutoSetTM | 26.5 | 2010 |
| S9 AutoSetTM with H5i Humidifier | 28.6 | 2010 |

One known RPT device used for treating sleep disordered breathing is the S9 Sleep Therapy System, manufactured by ResMed Limited. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

A range of artificial humidification devices and systems are known, however they may not fulfil the specialised requirements of a medical humidifier.

Medical humidifiers are used to increase humidity and/or temperature of the flow of air in relation to ambient air when required, typically where the patient may be asleep or resting (e.g. at a hospital). A medical humidifier for bedside placement may be small. A medical humidifier may be configured to only humidify and/or heat the flow of air delivered to the patient without humidifying and/or heating the patient's surroundings. Room-based systems (e.g. a sauna, an air conditioner, or an evaporative cooler), for example, may also humidify air that is breathed in by the patient, however those systems would also humidify and/or heat the entire room, which may cause discomfort to the occupants. Furthermore, medical humidifiers may have more stringent safety constraints than industrial humidifiers

While a number of medical humidifiers are known, they can suffer from one or more shortcomings. Some medical humidifiers may provide inadequate humidification, some are difficult or inconvenient to use by patients.

### 2.2.3.5 Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched gas are condensed. POCs seek to utilize their produced oxygen as efficiently as possible, in order to minimise weight, size, and power consumption. This may be achieved by delivering the oxygen as series of pulses, each pulse or “bolus” timed to coincide with the onset of inhalation. This therapy mode is known as pulsed oxygen delivery (POD) or demand mode, in contrast with traditional continuous flow delivery more suited to stationary oxygen concentrators.

### 2.2.3.6 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.7 Mandibular Repositioning

A mandibular repositioning device (MRD) or mandibular advancement device (MAD) is one of the treatment options for sleep apnea and snoring. It is an adjustable oral appliance available from a dentist or other supplier that holds the lower jaw (mandible) in a forward position during sleep. The MRD is a removable device that a patient inserts into their mouth prior to going to sleep and removes following sleep. Thus, the MRD is not designed to be worn all of the time. The MRD may be custom made or produced in a standard form and includes a bite impression portion designed to allow fitting to a patient's teeth. This mechanical protrusion of the lower jaw expands the space behind the tongue, puts tension on the pharyngeal walls to reduce collapse of the airway and diminishes palate vibration.

In certain examples a mandibular advancement device may comprise an upper splint that is intended to engage with or fit over teeth on the upper jaw or maxilla and a lower splint that is intended to engage with or fit over teeth on the upper jaw or mandible. The upper and lower splints are connected together laterally via a pair of connecting rods. The pair of connecting rods are fixed symmetrically on the upper splint and on the lower splint.

In such a design the length of the connecting rods is selected such that when the MRD is placed in a patient's mouth the mandible is held in an advanced position. The length of the connecting rods may be adjusted to change the level of protrusion of the mandible. A dentist may determine a level of protrusion for the mandible that will determine the length of the connecting rods.

Some MRDs are structured to push the mandible forward relative to the maxilla while other MADs, such as the ResMed Narval CC™ MRD are designed to retain the mandible in a forward position. This device also reduces or minimises dental and temporo-mandibular joint (TMJ) side effects. Thus, it is configured to minimises or prevent any movement of one or more of the teeth.

### 2.2.3.8 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

| | | A-weighted | A-weighted | |
|---|---|---|---|---|
| | | sound power | sound pressure | |
| | Mask | level dB(A) | dB(A) | Year |
| Mask name | type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| MirageTM (*) | | | | |
| ResMed | nasal | 36 (3) | 28 (3) | 2000 |
| UltraMirageTM | | | | |
| ResMed | nasal | 32 (3) | 24 (3) | 2002 |
| Mirage | | | | |
| ActivaTM | | | | |
| ResMed | nasal | 30 (3) | 22 (3) | 2008 |
| Mirage | | | | |
| MicroTM | | | | |
| ResMed | nasal | 29 (3) | 22 (3) | 2008 |
| MirageTM | | | | |
| SoftGel | | | | |
| ResMed | nasal | 26 (3) | 18 (3) | 2010 |
| MirageTM FX | | | | |
| ResMed | nasal | 37 | 29 | 2004 |
| Mirage | pillows | | | |
| SwiftTM (*) | | | | |
| ResMed | nasal | 28 (3) | 20 (3) | 2005 |
| Mirage | pillows | | | |
| SwiftTM II | | | | |
| ResMed | nasal | 25 (3) | 17 (3) | 2008 |
| Mirage | pillows | | | |
| SwiftTM LT | | | | |
| ResMed AirFit | nasal | 21 (3) | 13 (3) | 2014 |
| P10 | pillows | | | |

((*) one specimen only, measured using test method specified in ISO 3744 in CPAP mode at 10 cmH2O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at |
| Walter Broadly Litter Hog: | | 1 m distance |
| B+ Grade | | |
| Conversational speech | 60 | 1 m distance |
| Average home | 50 | |
| Quiet library | 40 | |
| Quiet bedroom at night | 30 | |
| Background in TV studio | 20 | |

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a system for sensing parameters associated with a respiratory therapy (“RPT”) system, the system comprising a first circuit board assembly having at least one control element, a second circuit board assembly having at least one sensor, wherein the second circuit board assembly is configured to be coupled to a patient interface of the RPT system, such that the sensor is configured to sense a parameter within a plenum chamber of the patient interface, and a connector that electrically connects the first circuit board assembly to the second circuit board assembly.

In examples of the form of the present technology:
a. in a configuration in which the second circuit board is coupled to the patient interface, the sensor is further configured to sense a parameter of an atmosphere outside of the plenum chamber;
b. the sensor includes a pressure sensor, a humidity sensor, a temperature sensor, or a CO₂ sensor;
c. a grommet houses the second circuit board assembly;
d. a portion of the patient interface defining the plenum chamber includes an opening in communication with the plenum chamber and accommodating the grommet;
e. the grommet forms a seal with the plenum chamber when disposed in the opening;
f. the grommet defines a lumen;
g. in a configuration in which the grommet is disposed within the opening, the lumen is in communication with an atmosphere outside of the plenum chamber;
h. the sensor is at least partially disposed within the lumen;
i. the first circuit board assembly is configured to be attached to a strap of the patient interface;
j. when the second circuit board is coupled to the patient interface, the first circuit board is located outside of the plenum chamber;
k. the system further includes a docking station for removably receiving the first circuit board assembly;
l. the docking station includes circuitry for charging a battery of the first circuit board assembly;
m. the docking station includes circuitry for communicating with an external device;
n. the circuitry for communicating with an external device includes a USB module;
o. the first circuit board assembly includes a housing having at least one hole;
p. the hole is configured to receive a pin for connecting to a first circuit board of the first circuit board assembly;
q. the connector includes an I²C bus;
r. the second circuit board assembly is configured to be removable from the connector, the system further comprising a third circuit board assembly configured to be coupled to the connector following removal of the second circuit board assembly; and/or
s. at least one of the first circuit board assembly or the second circuit board assembly includes circuitry for wirelessly communicating with an external device.

One form of the present technology comprises a system for sensing parameters associated with a respiratory therapy (“RPT”) system, the system comprising a circuit board and at least one sensor mounted on the circuit board, wherein the circuit board is configured to be coupled to a patient interface of the RPT system, such that the at least one sensor is configured to sense a parameter within a plenum chamber of the patient interface and a parameter of an atmosphere outside of the plenum chamber.

In an example, a portion of the patient interface defining the plenum chamber includes an opening in communication with the plenum chamber, wherein the circuit board is coupled to a grommet, and wherein the opening is configured to accommodate the grommet.

In another example, the circuit board is configured to be mounted within the plenum chamber, and wherein the sensor is configured to sense the parameter of the atmosphere via an opening of a vent in fluid communication with the plenum chamber.

Another form of the present technology comprises a system for sensing parameters associated with a respiratory therapy (“RPT”) system, the system comprising a circuit board disposed on a grommet and a sensor mounted to the circuit board, wherein a portion of the patient interface defining a plenum chamber includes an opening in communication with the plenum chamber and accommodating the grommet.

Another form of the present technology comprises a respiratory therapy system comprising:
a respiratory therapy (“RPT”) device for providing a positive pressure flow of gas to an airway of a patient, the RPT device including:
   a patient interface to interface the RPT device to the patient, and
   respiratory equipment to supply the positive pressure flow at flow parameters to the patient interface; and
a circuit board assembly having at least one sensor, wherein the circuit board assembly is coupled to the patient interface, such that the sensor is configured to sense a parameter within a plenum chamber of the patient interface,
wherein at least one of the flow parameters is adjusted based on the parameter sensed by the sensor.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

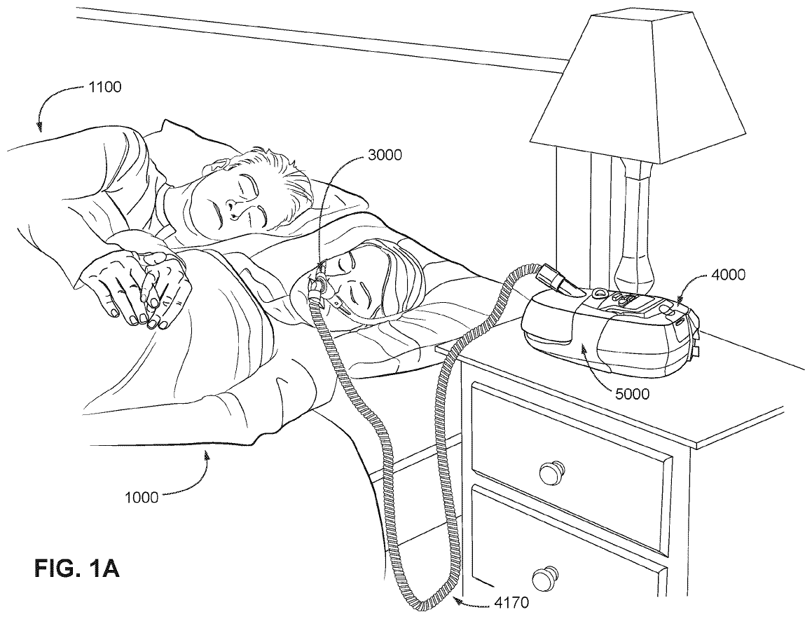

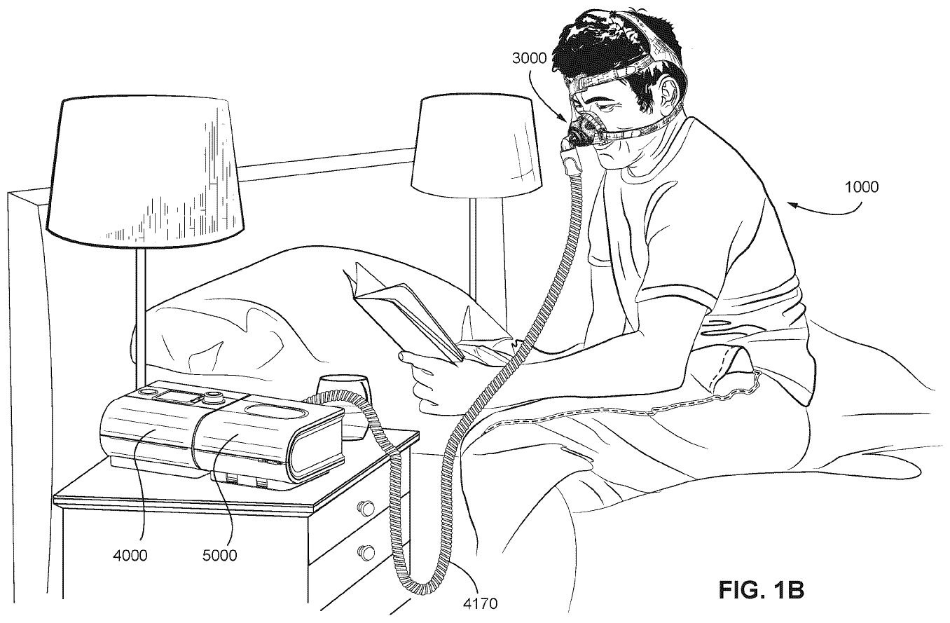

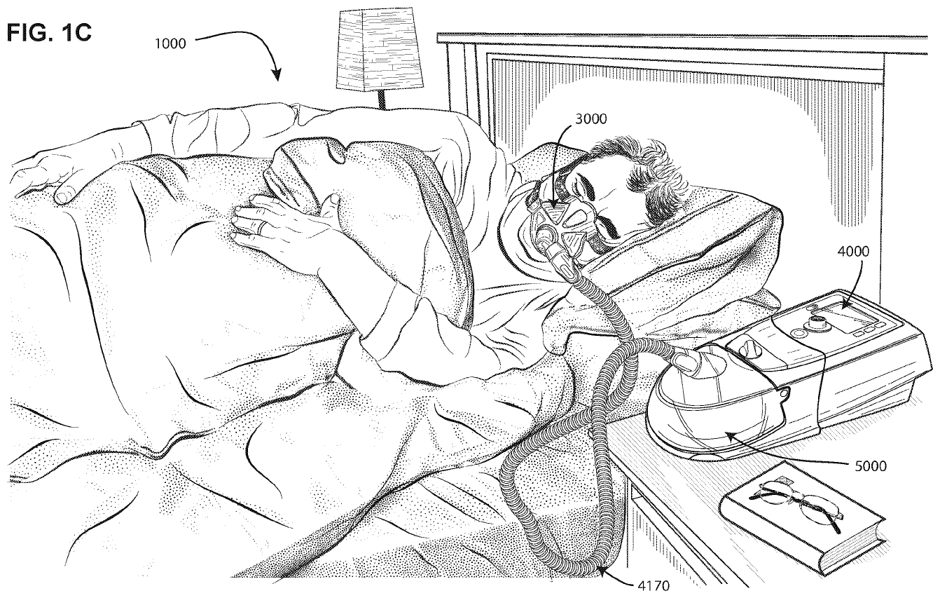

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

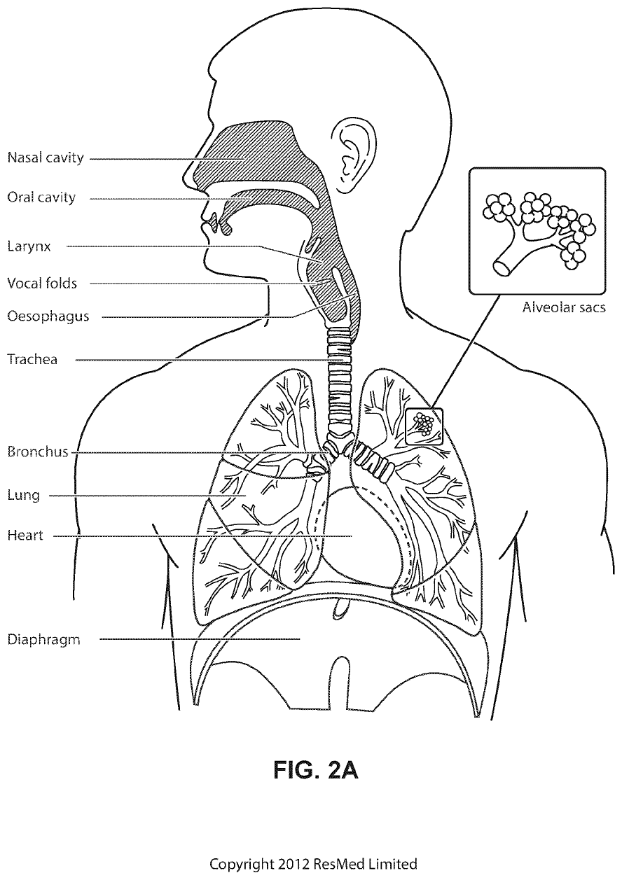

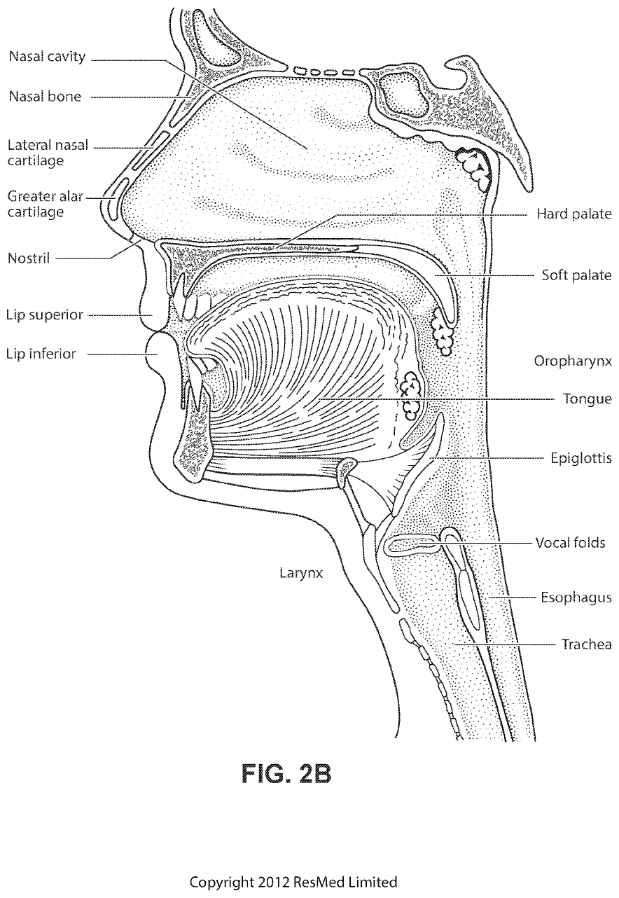

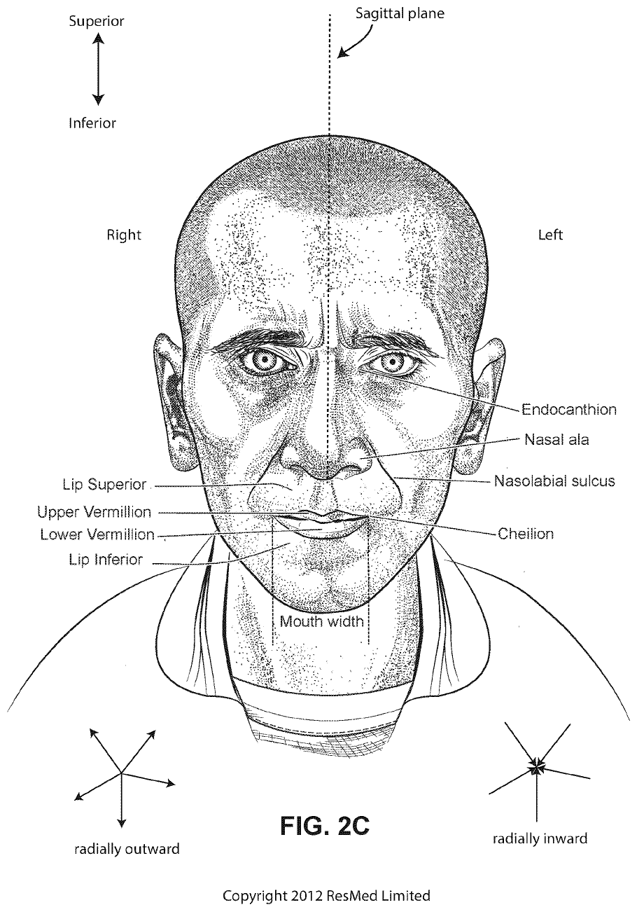

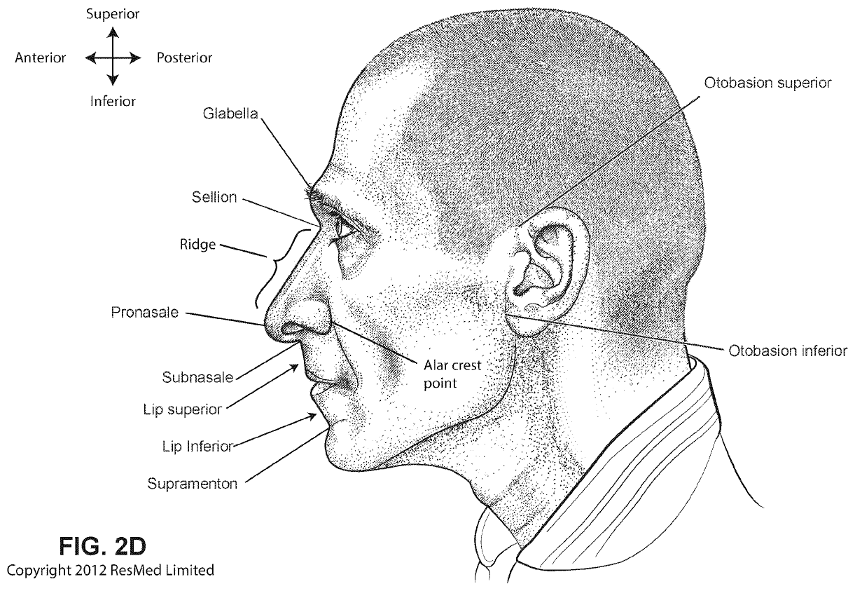

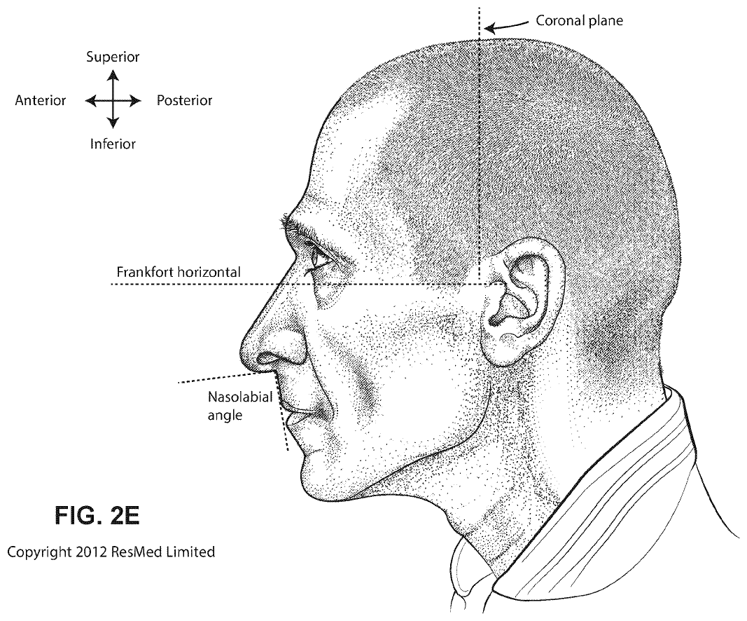

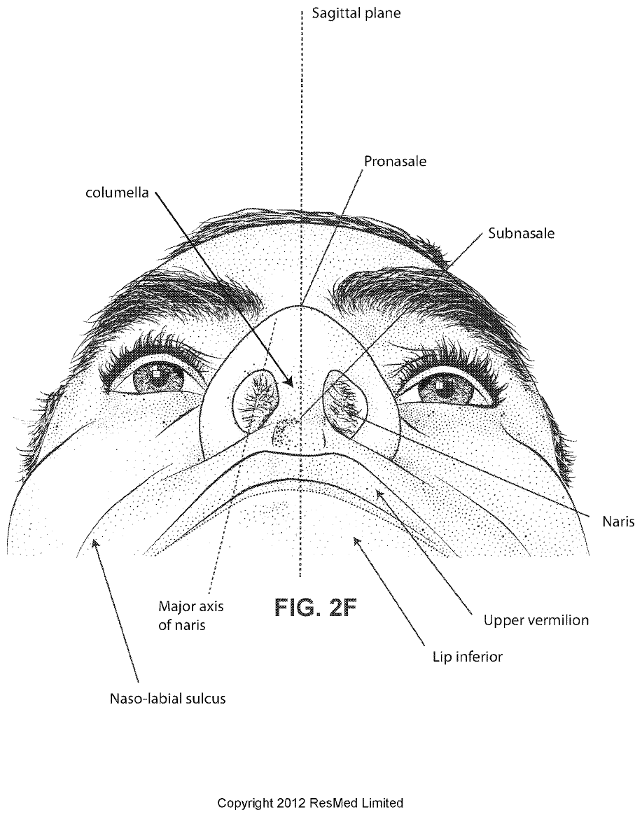

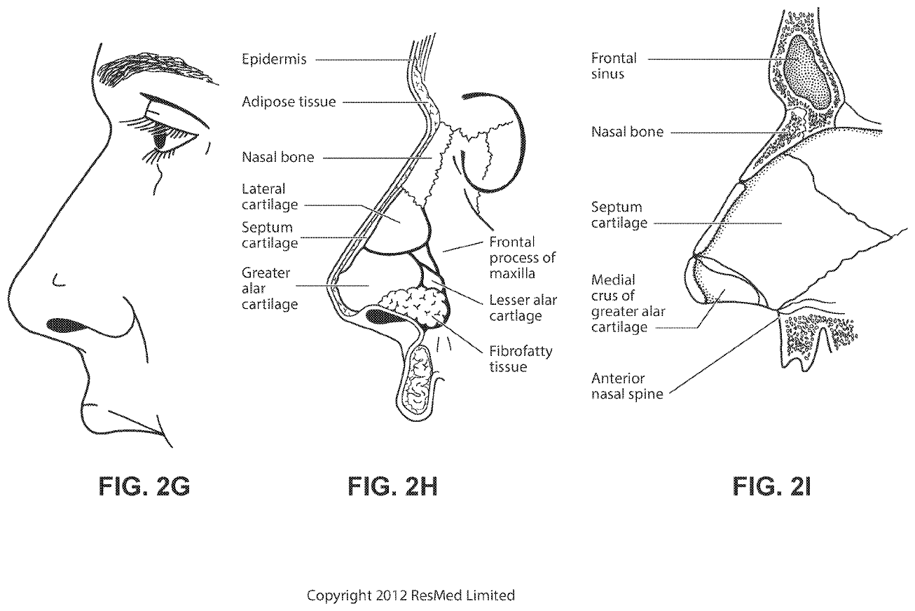

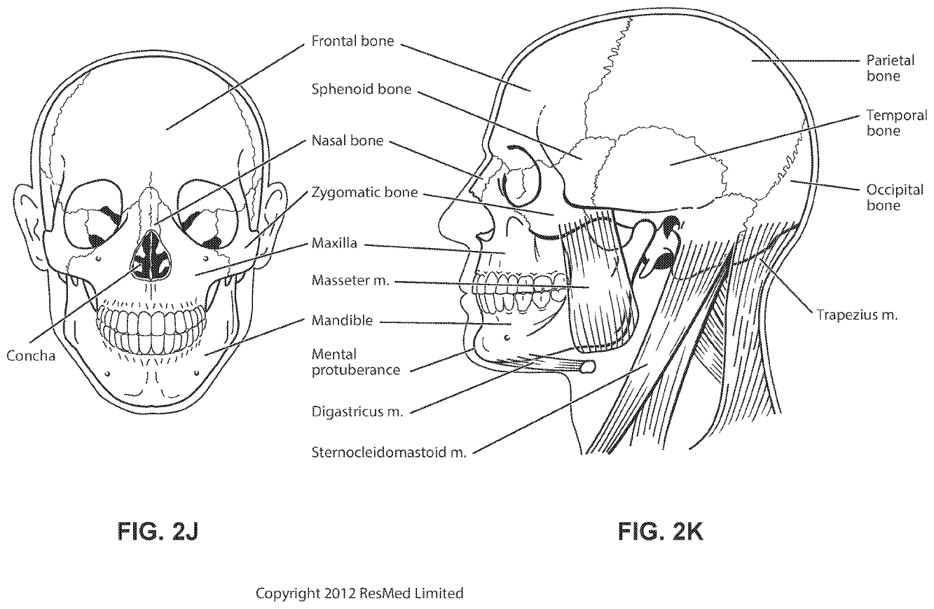

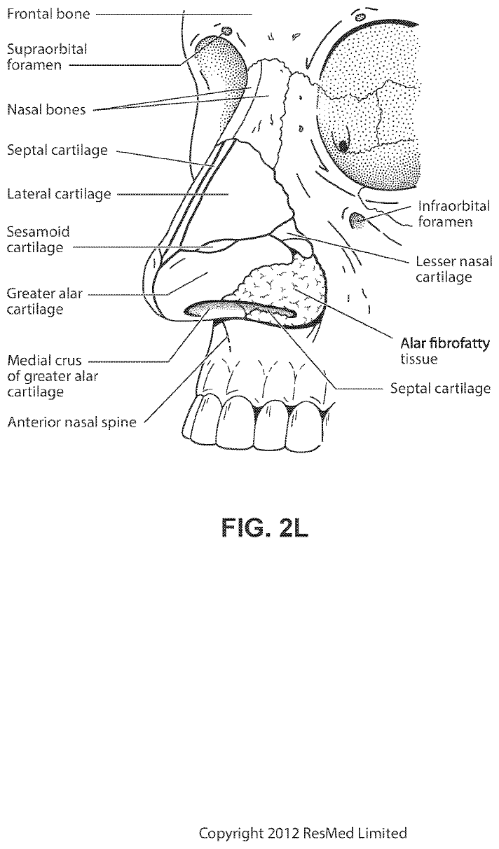

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideopezius.

FIG. 2L shows an anterolateral view of a nose.

### 4.3 Patient Interface

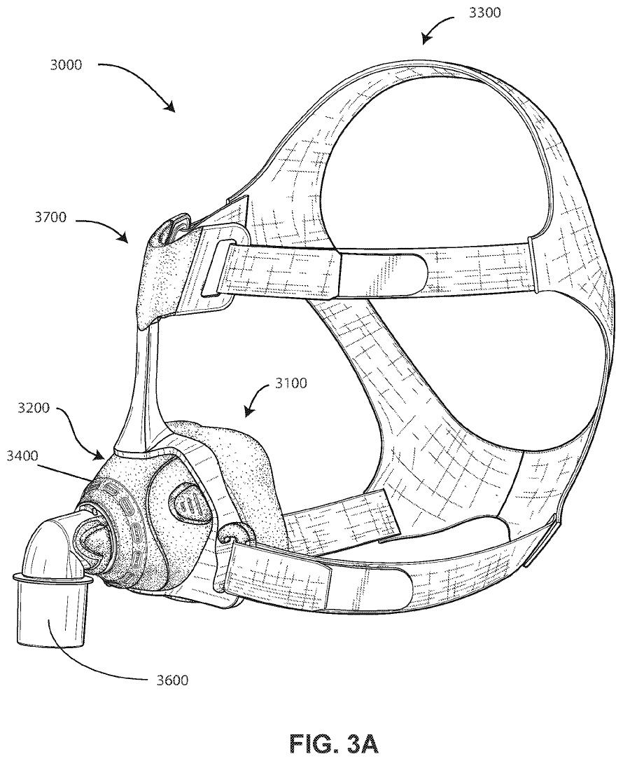

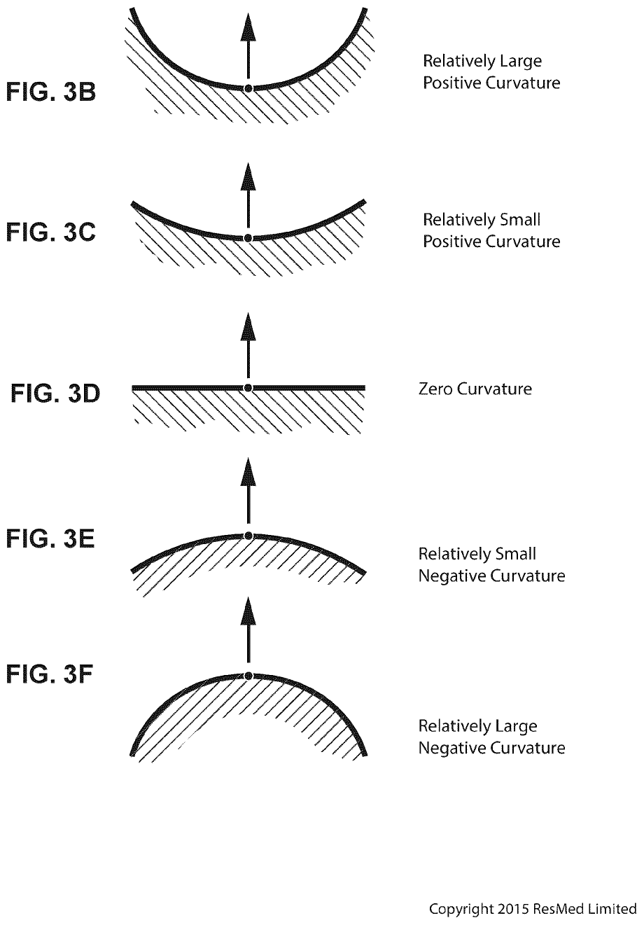

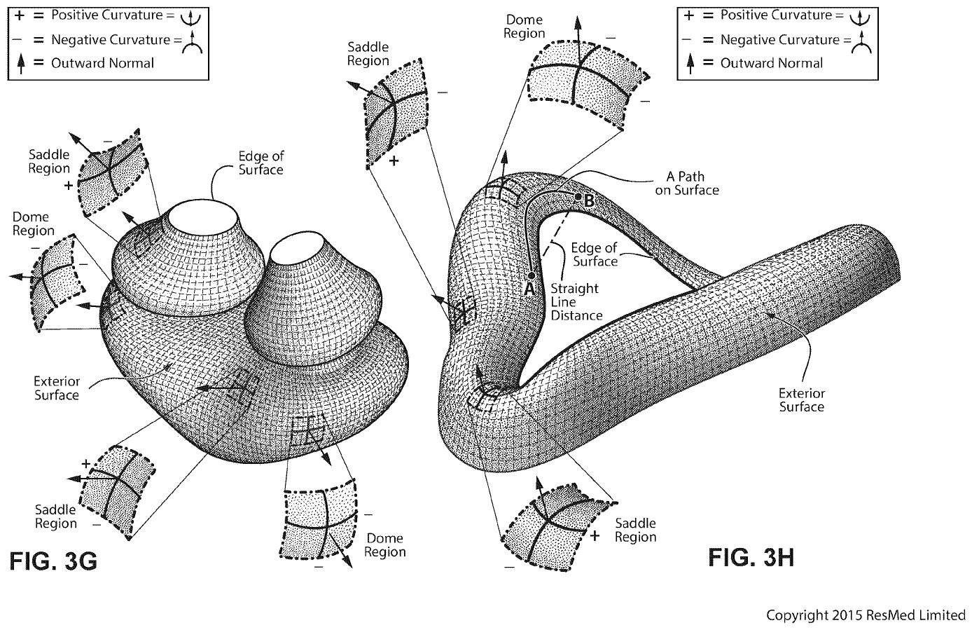

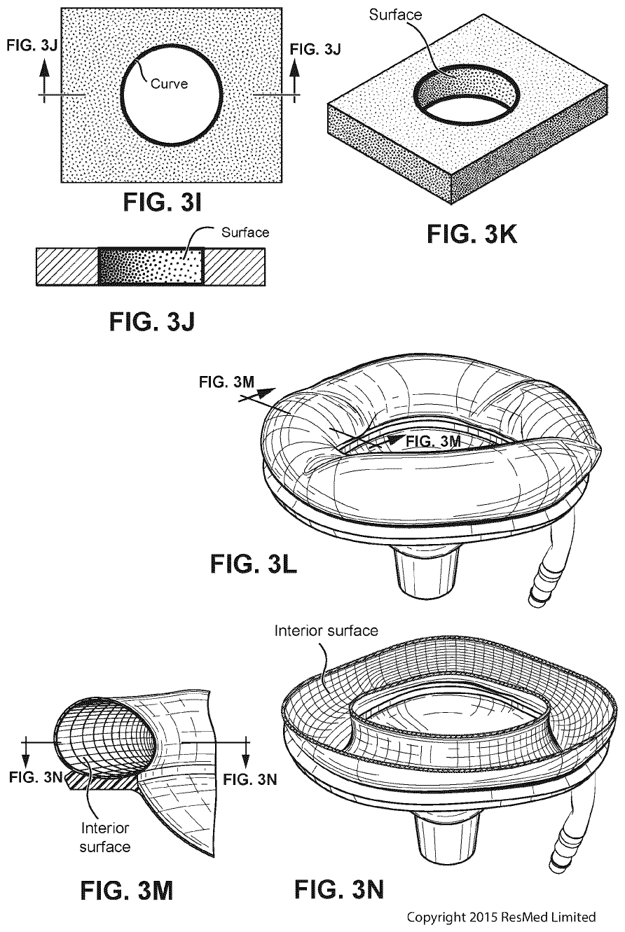

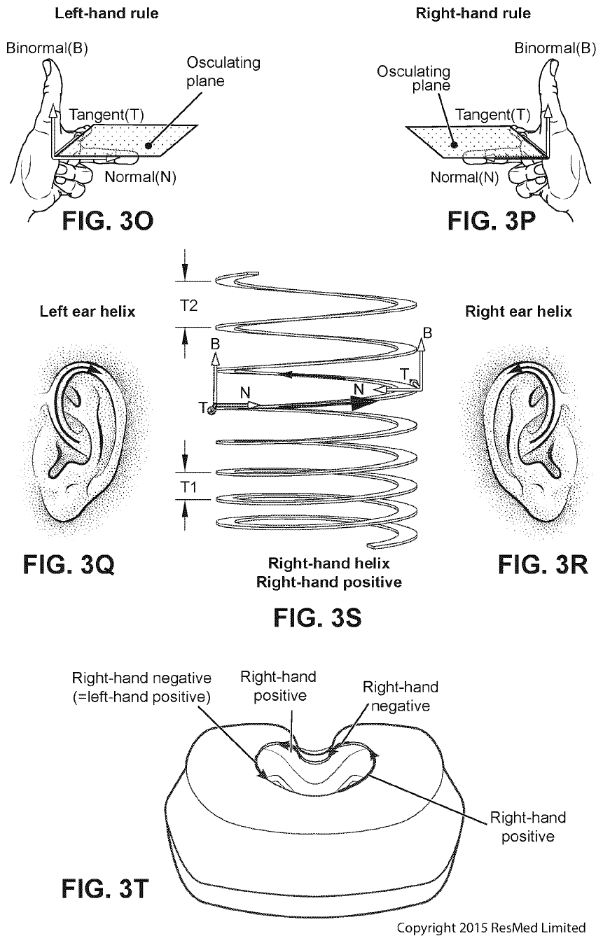

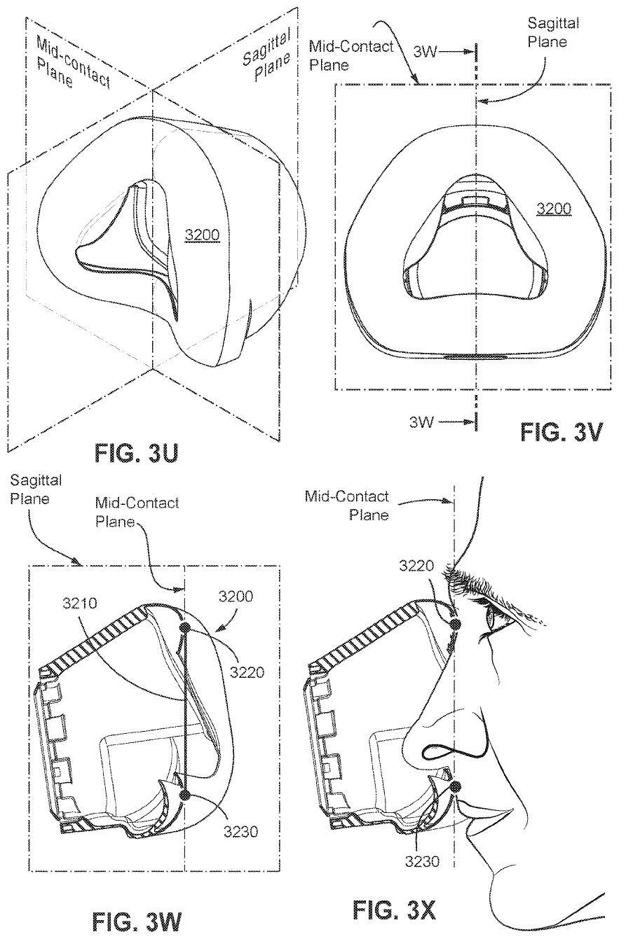

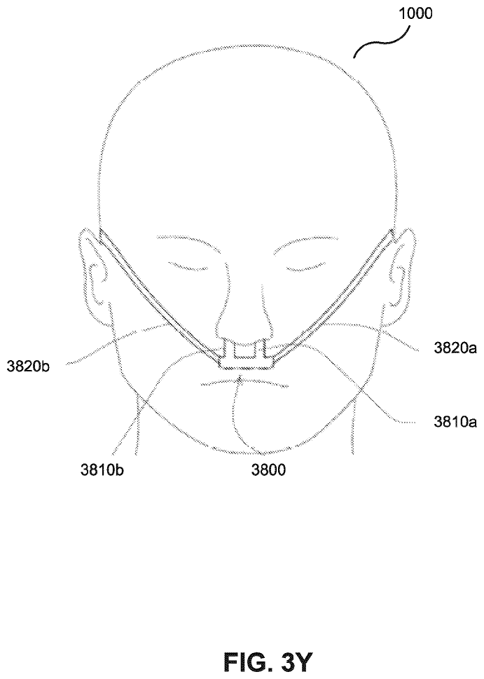

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

FIG. 3Y shows a patient interface in the form of a nasal cannula in accordance with one form of the present technology.

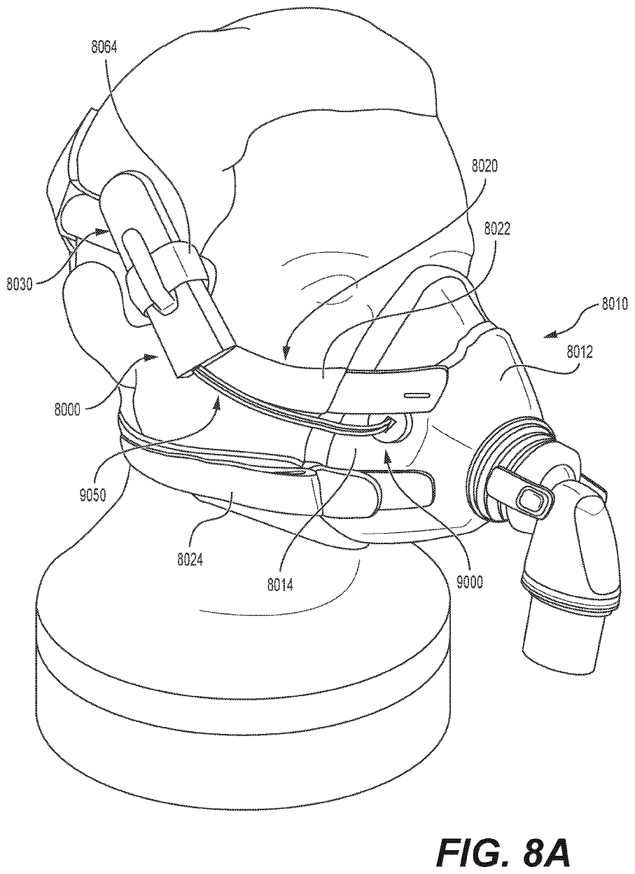

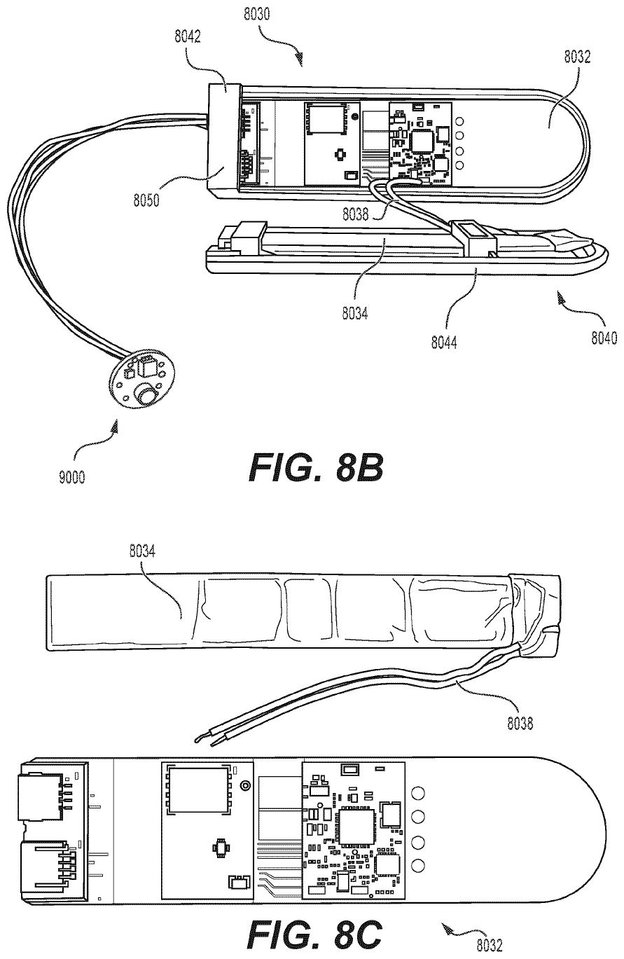

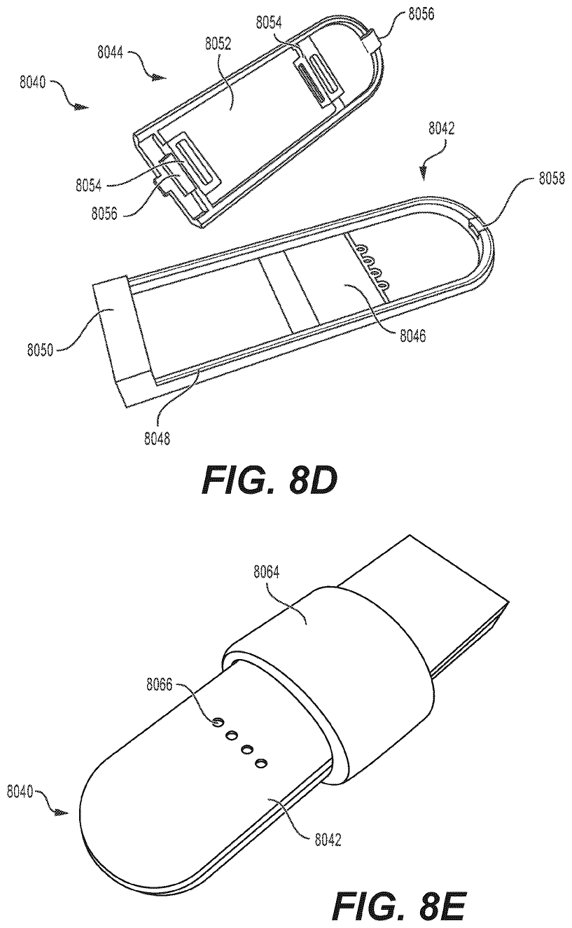

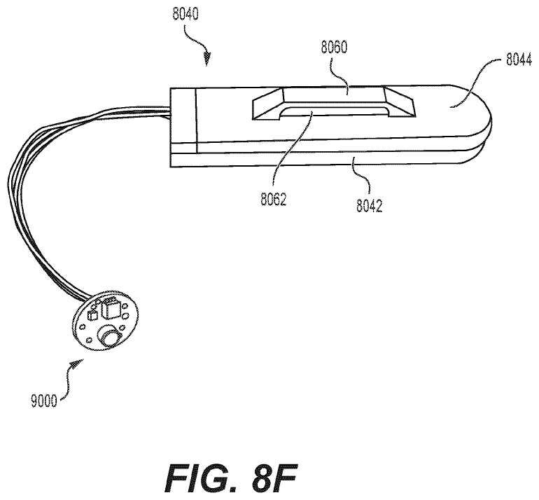

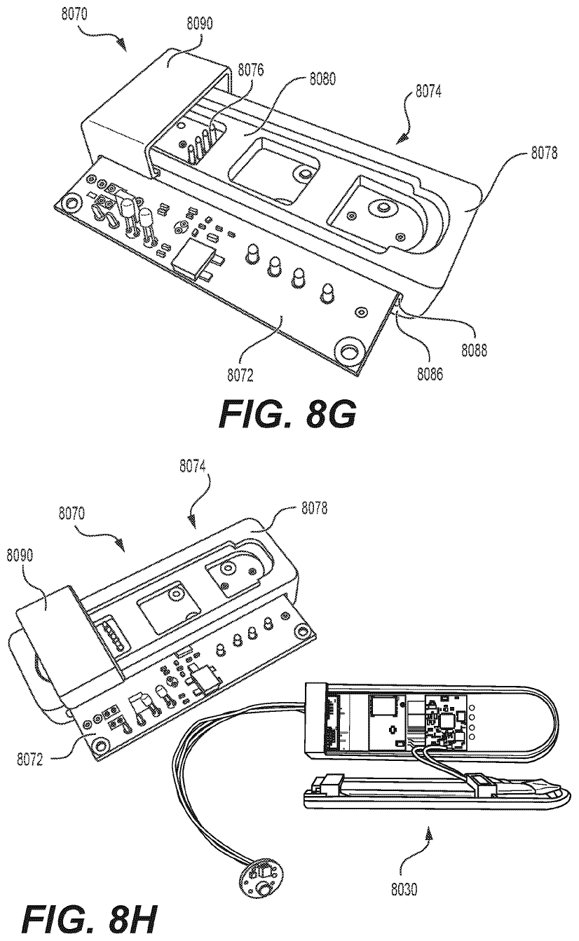

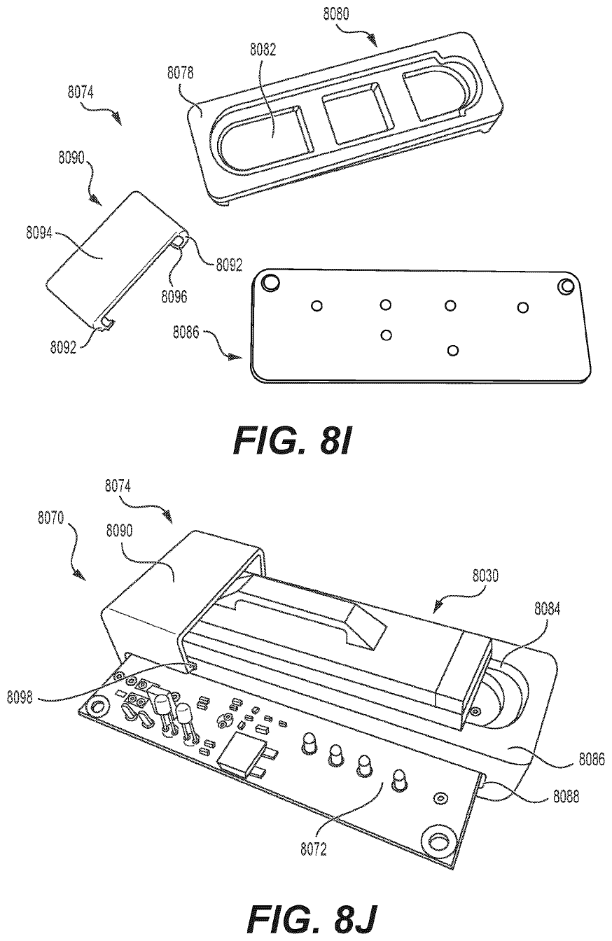

FIG. 8A shows a patient interface 8010 having a sensing kit 8000 thereon, in accordance with one form of the present technology.

FIG. 8B depicts the sensing kit 8000.

FIG. 8C depicts elements of a first circuit board assembly 8030 of the sensing kit 8000.

FIG. 8D shows a housing 8040 of the first circuit board assembly 8030.

FIG. 8E shows the housing 8040 of the first circuit board assembly 8030.

FIG. 8F shows another view of the sensing kit 8000.

FIG. 8G shows a docking station 8070 for the sensing kit 8000.

FIG. 8H depicts the docking station 8070 and the sensing kit 8000.

FIG. 8I depicts a housing 8074 of the docking station 8070.

FIG. 8J depicts the first circuit board assembly 8030 docked in the docking station 8070.

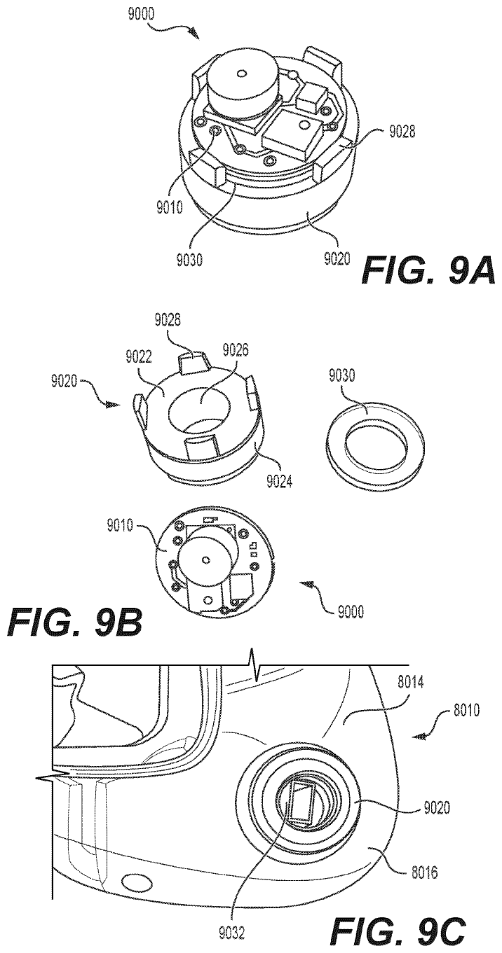

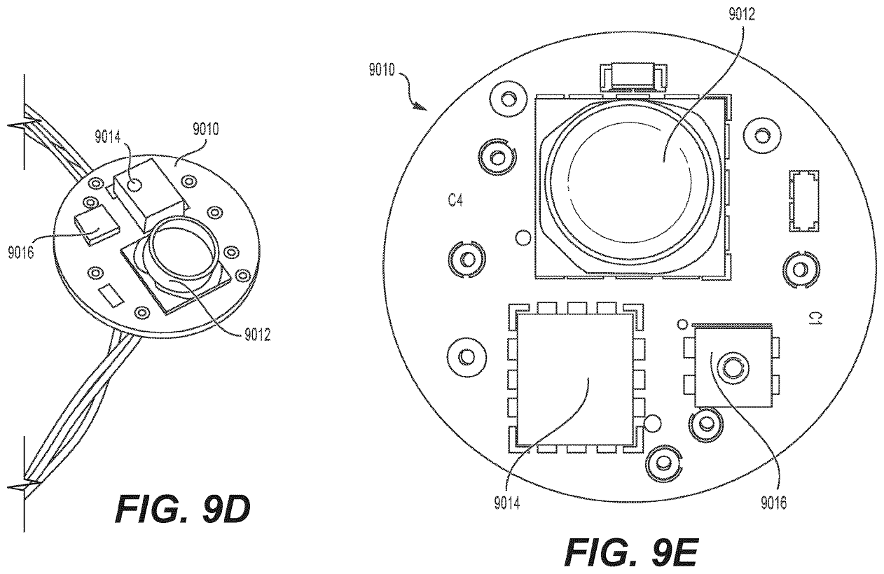

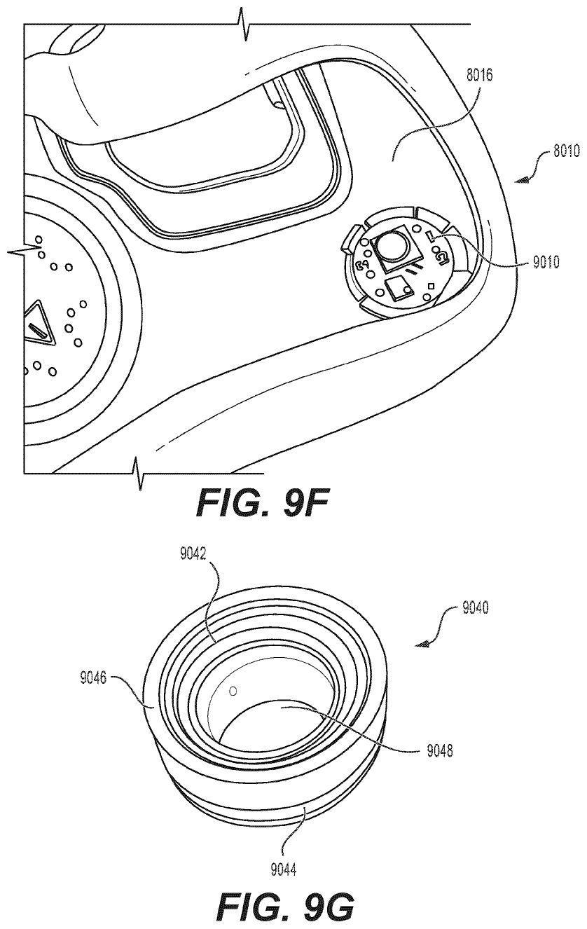

FIG. 9A depicts a second circuit board assembly 9000 of the sensing kit 8000.

FIG. 9B depicts the components of the second circuit board assembly 9000 in a disassembled state.

FIG. 9C depicts a patient interface 8010 having the second circuit board assembly 9000 thereon.

FIG. 9D depicts a second circuit board 9010 of the second circuit board assembly 9000.

FIG. 9E is a schematic diagram of the second circuit board 9010.

FIG. 9F shows a patient interface 8010 having the second circuit board assembly 9000 thereon.

FIG. 9G depicts a grommet 9040 for installing the second circuit board assembly 9000 on the patient interface 8010.

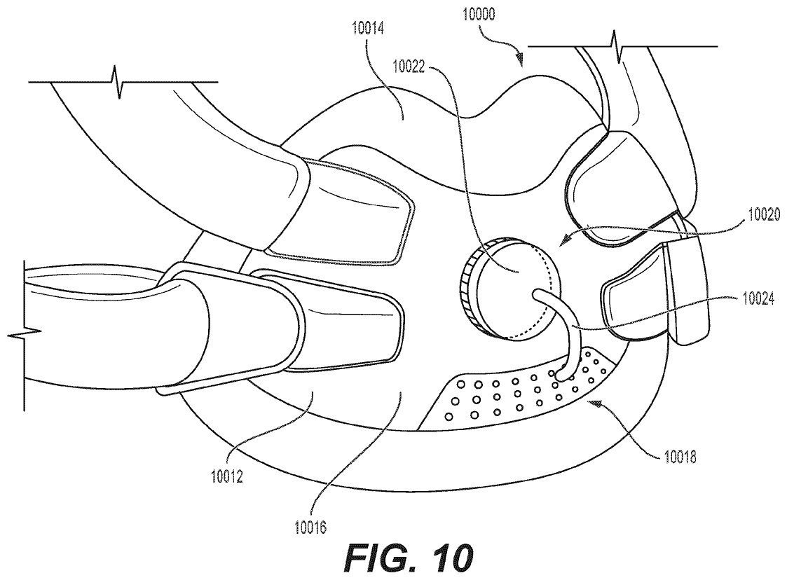

FIG. 10 depicts an alternative patient interface 10000 having a second circuit board assembly 10020 thereon.

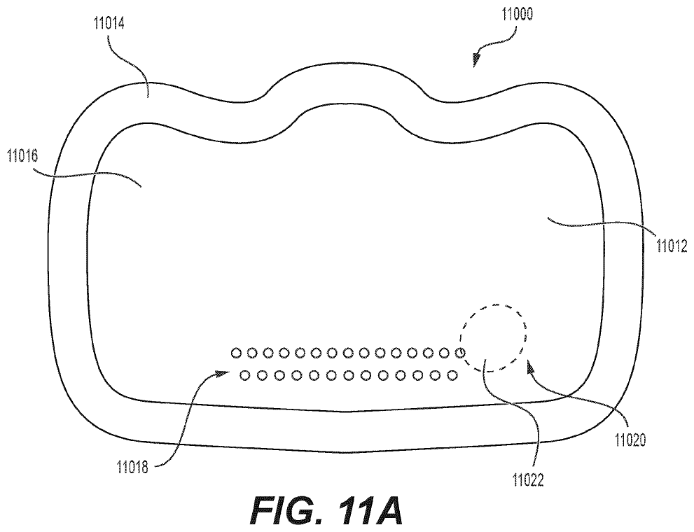

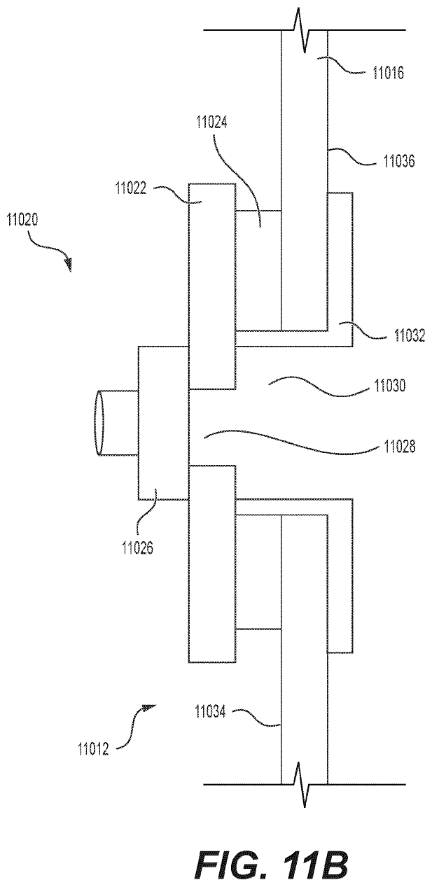

FIG. 11A depicts an alternative patient interface 11000 having a second circuit board assembly 11020 thereon.

FIG. 11B depicts a detail of a portion of the alternative patient interface 11020.

### 4.4 RPT Device

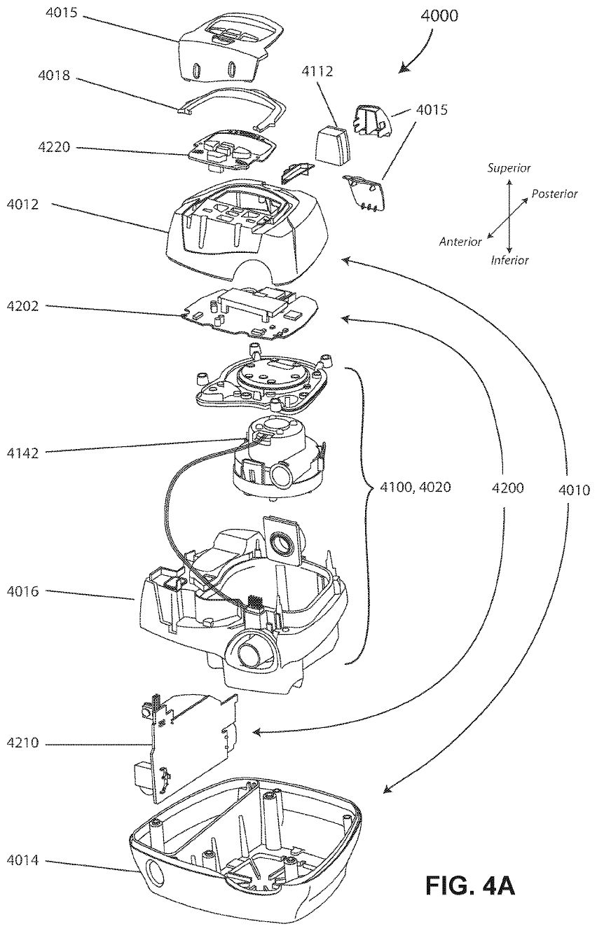

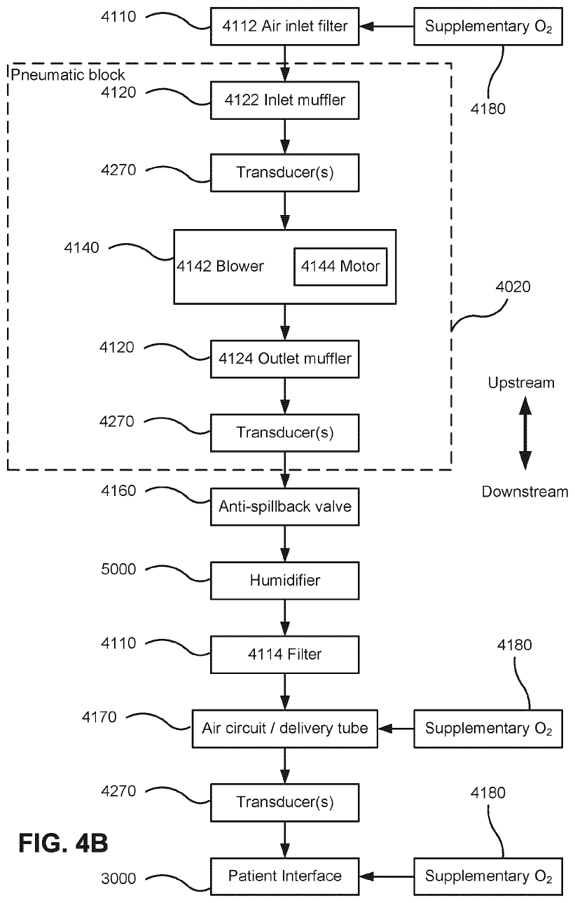

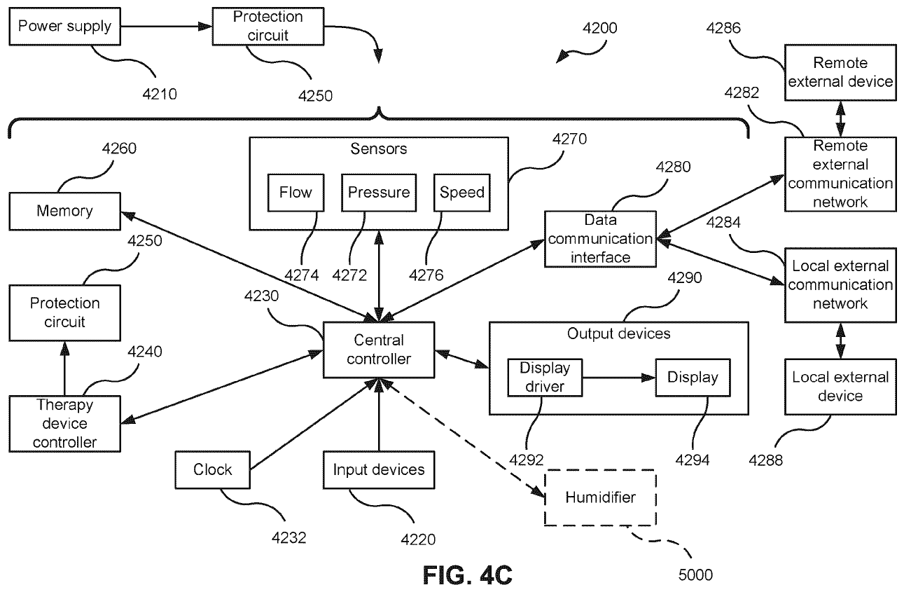

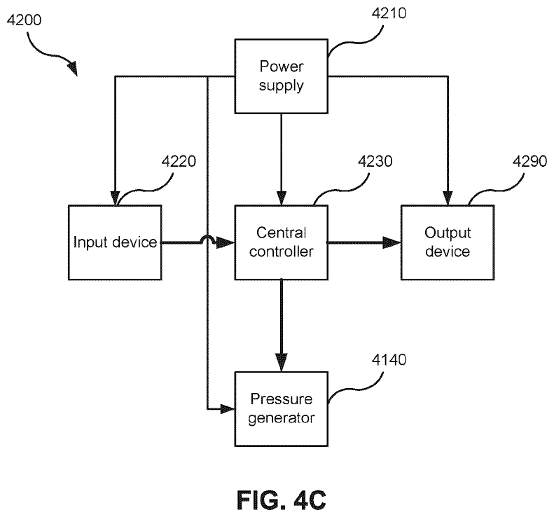

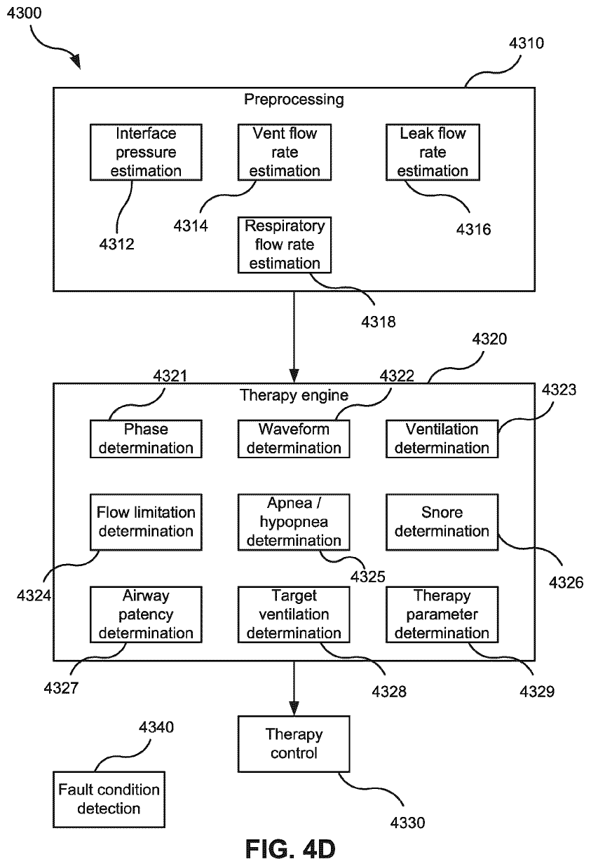

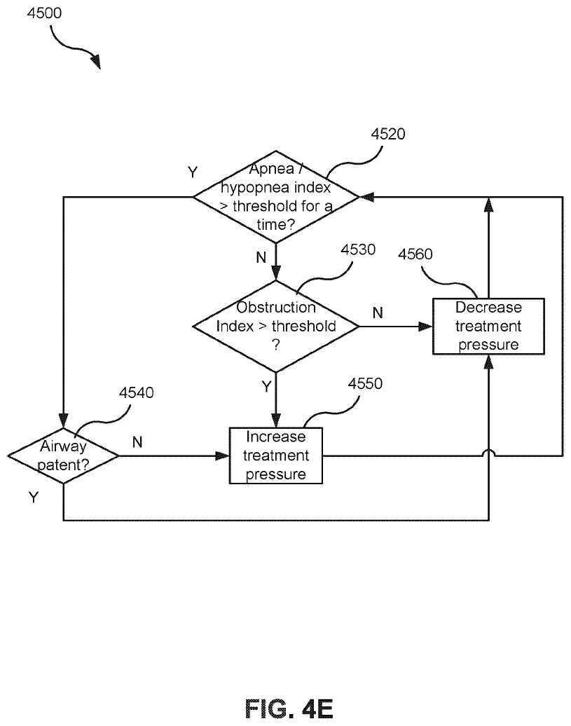

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RPT device in accordance with one form of the present technology.

FIG. 4D is a schematic diagram of the algorithms implemented in an RPT device in accordance with one form of the present technology.

FIG. 4E is a flow chart illustrating a method carried out by the therapy engine module of FIG. 4D in accordance with one form of the present technology.

### 4.5 Humidifier

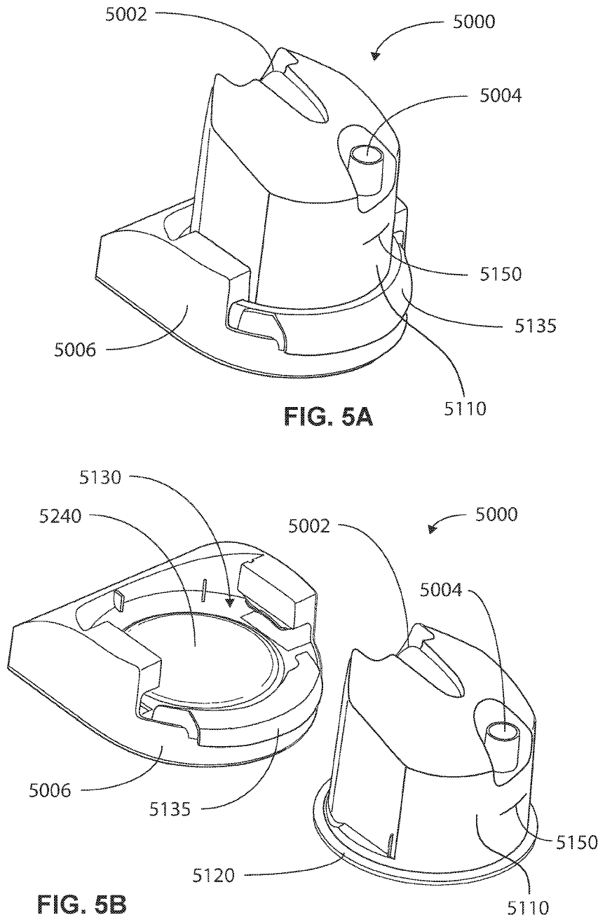

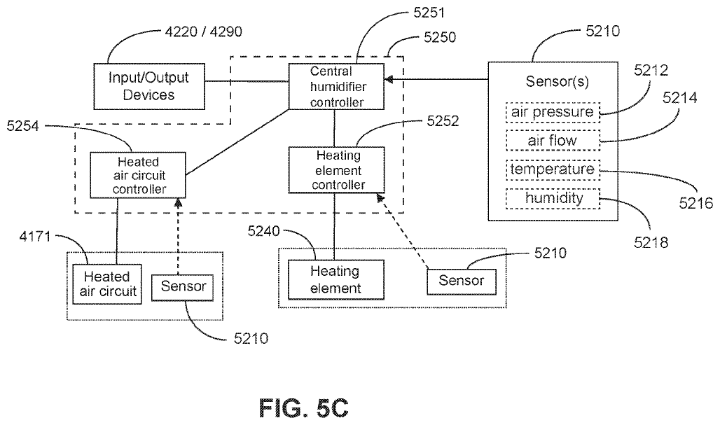

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

FIG. 5C shows a schematic of a humidifier in accordance with one form of the present technology.

### 4.6 Breathing Waveforms

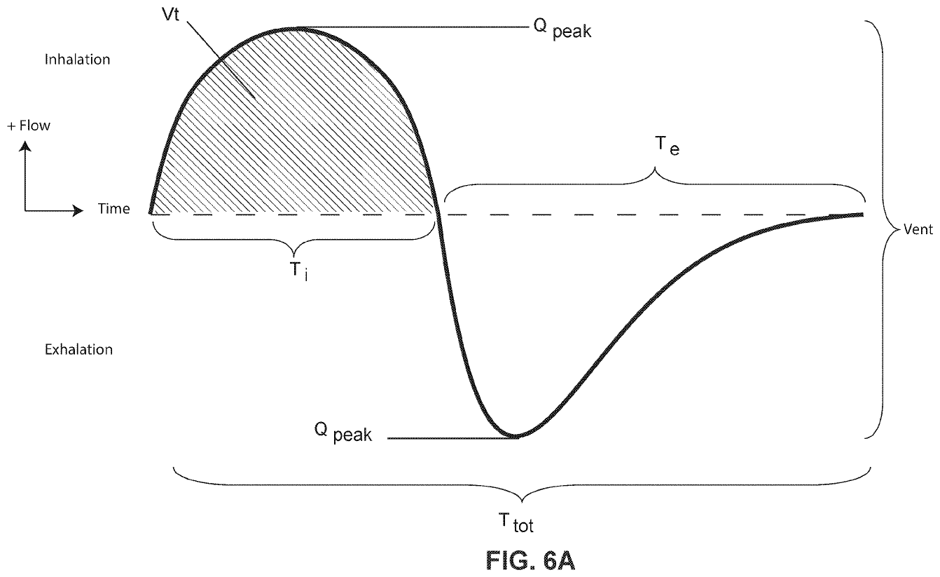

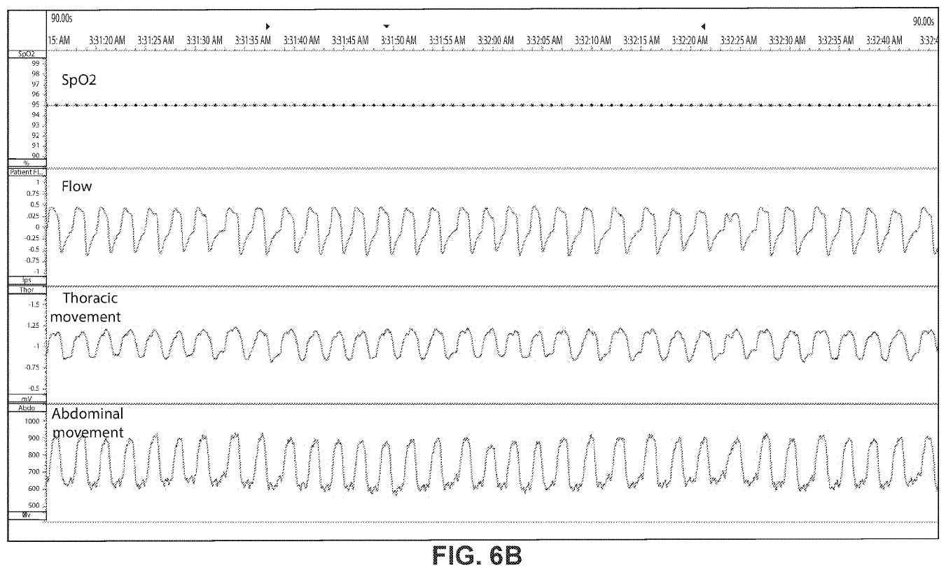

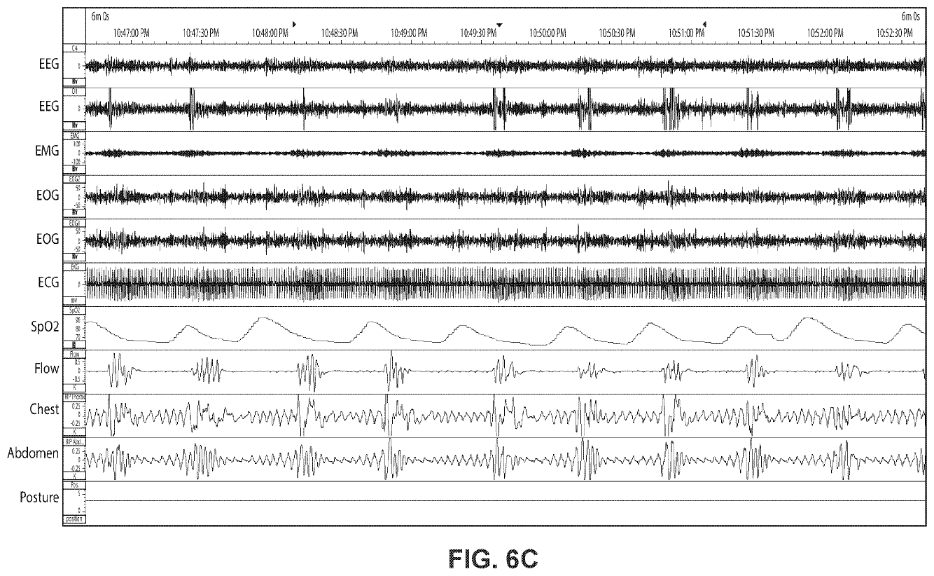

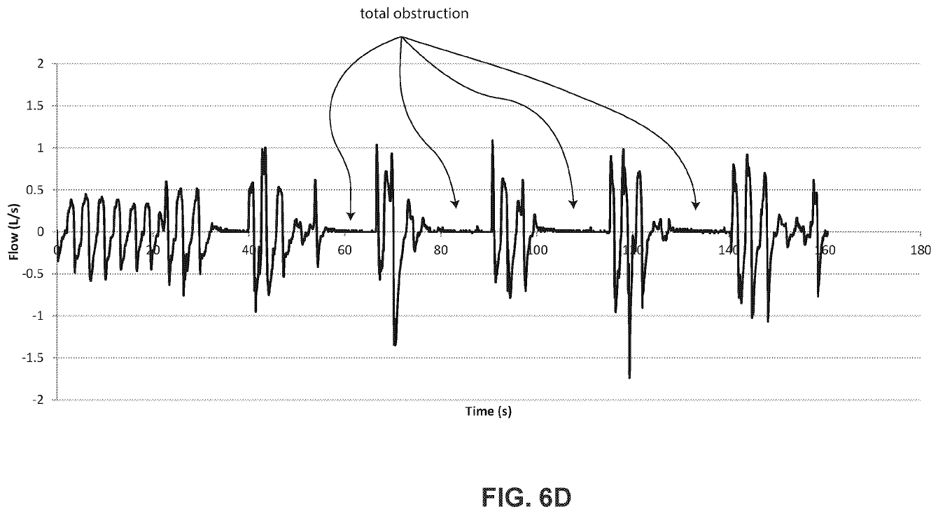

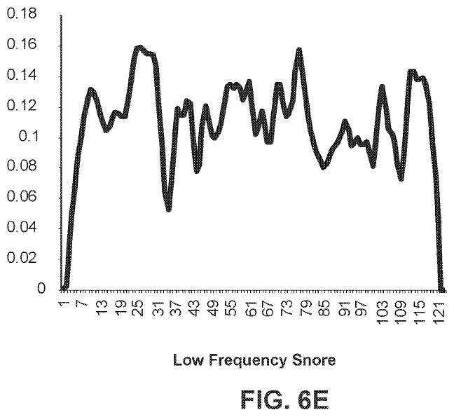

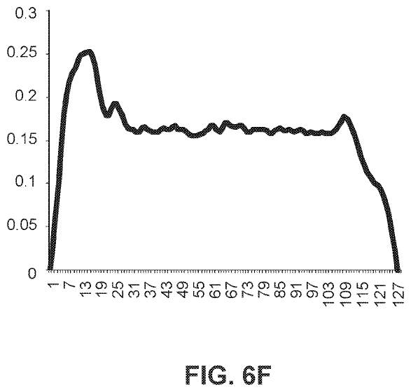

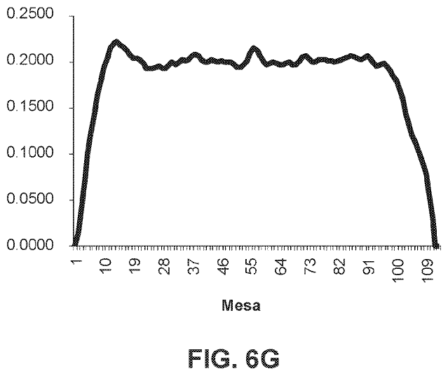

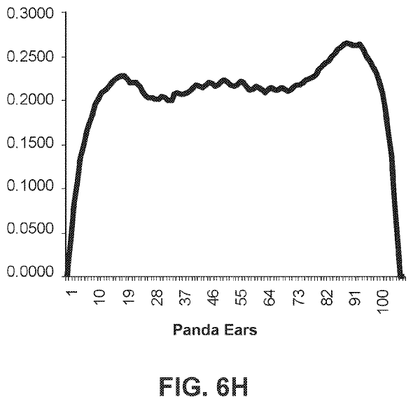

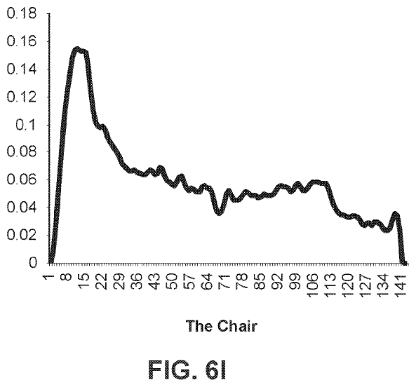

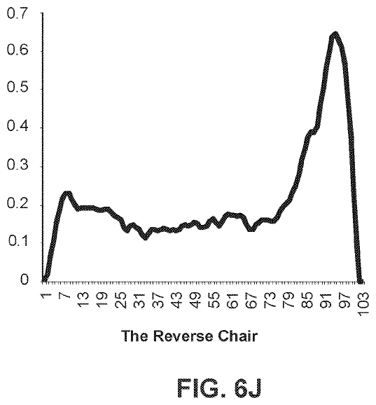

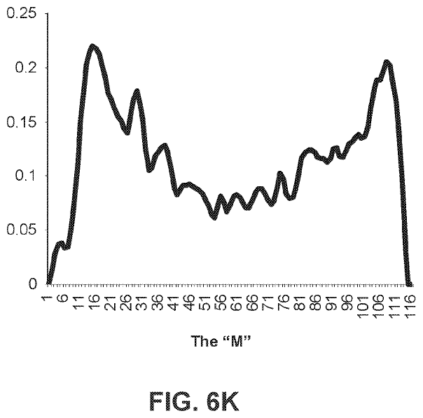

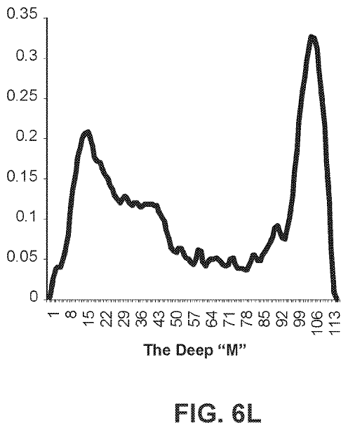

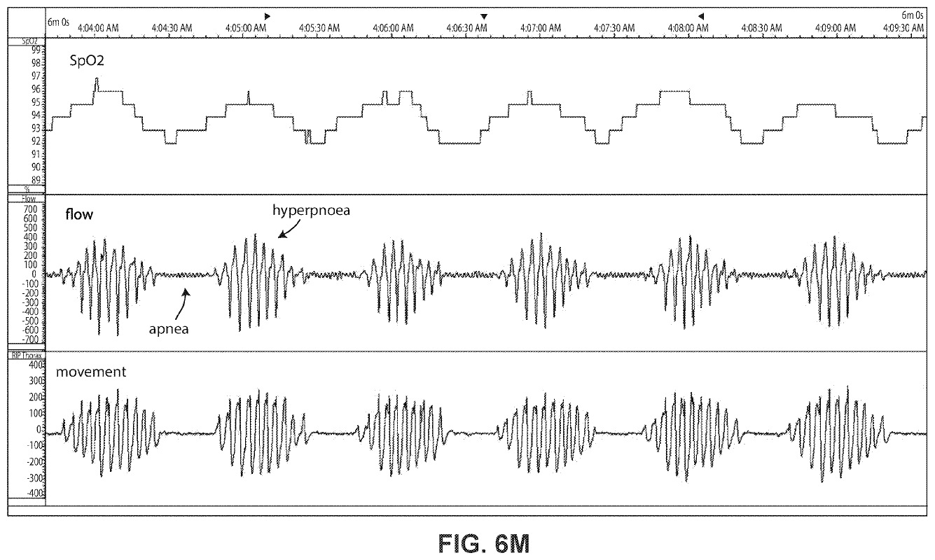

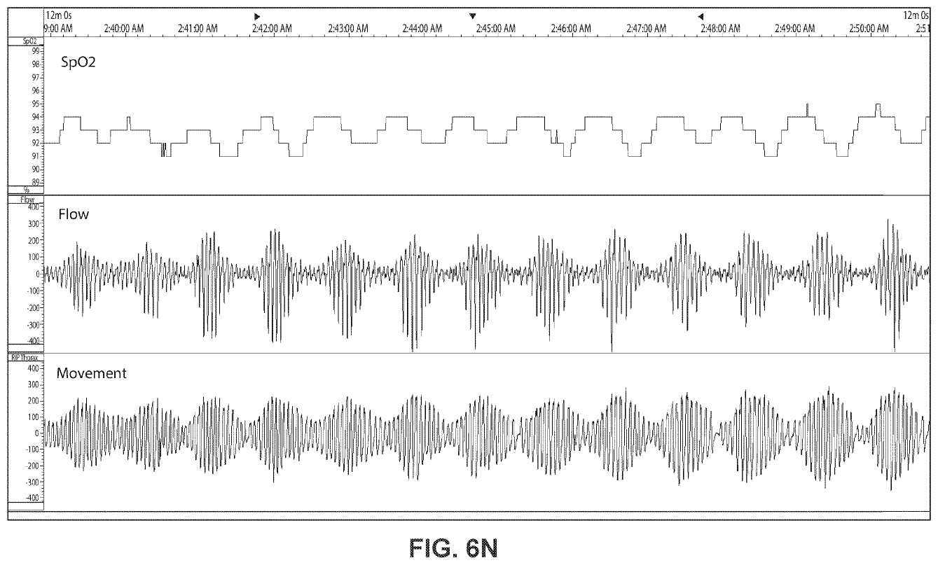

FIG. 6A shows a model typical breath waveform of a person while sleeping.

FIG. 6B shows selected polysomnography channels (pulse oximetry, flow rate, thoracic movement, and abdominal movement) of a patient during non-REM sleep breathing normally over a period of about ninety seconds.

FIG. 6C shows polysomnography of a patient before treatment.

FIG. 6D shows patient flow rate data where the patient is experiencing a series of total obstructive apneas.

FIG. 6E shows a scaled inspiratory portion of a breath where the patient is experiencing low frequency inspiratory snore.

FIG. 6F shows a scaled inspiratory portion of a breath where the patient is experiencing an example of flattened inspiratory flow limitation.

FIG. 6G shows a scaled inspiratory portion of a breath where the patient is experiencing an example of “mesa” flattened inspiratory flow limitation.

FIG. 6H shows a scaled inspiratory portion of a breath where the patient is experiencing an example of “panda ears” inspiratory flow limitation.

FIG. 6I shows a scaled inspiratory portion of a breath where the patient is experiencing an example of “chair” inspiratory flow limitation.

FIG. 6J shows a scaled inspiratory portion of a breath where the patient is experiencing an example of “reverse chair” inspiratory flow limitation.

FIG. 6K shows a scaled inspiratory portion of a breath where the patient is experiencing an example of “M-shaped” inspiratory flow limitation.

FIG. 6L shows a scaled inspiratory portion of a breath where the patient is experiencing an example of severely “M-shaped” inspiratory flow limitation.

FIG. 6M shows patient data from a patient with Cheyne-Stokes respiration.

FIG. 6N shows patient data from a patient with another example of Cheyne-Stokes respiration, using the same three channels as in FIG. 6M.

### 4.7 Screening, Diagnosis and Monitoring Systems

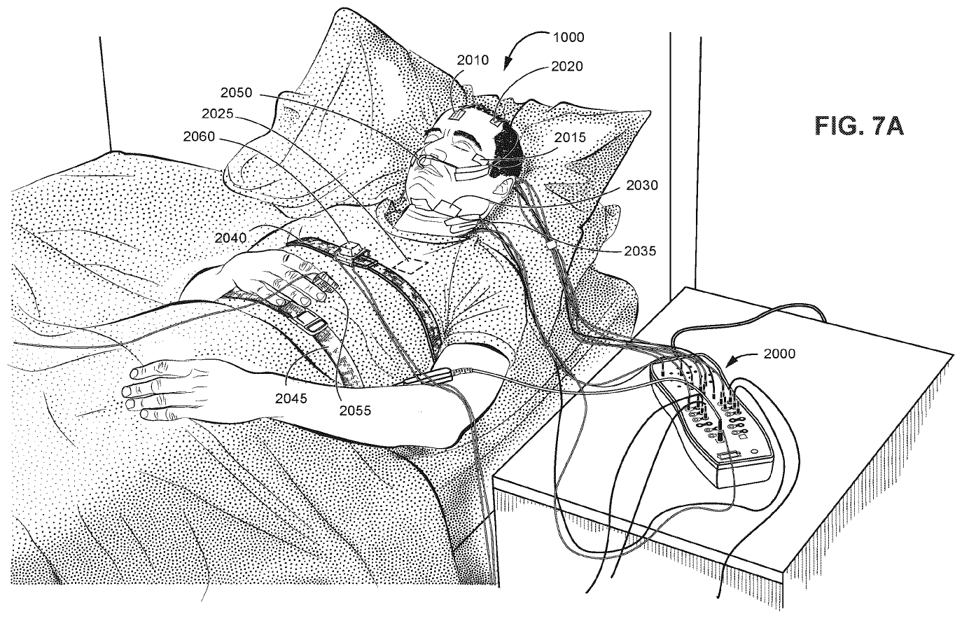

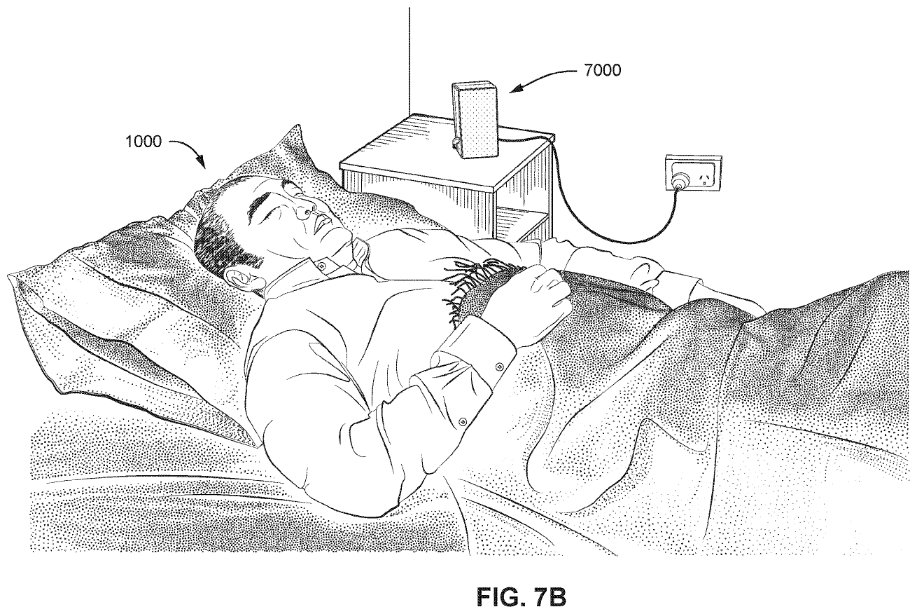

FIG. 7A shows a patient undergoing polysomnography (PSG). The patient is sleeping in a supine sleeping position.

FIG. 7B shows a monitoring apparatus for monitoring the condition of a patient. The patient is sleeping in a supine sleeping position.

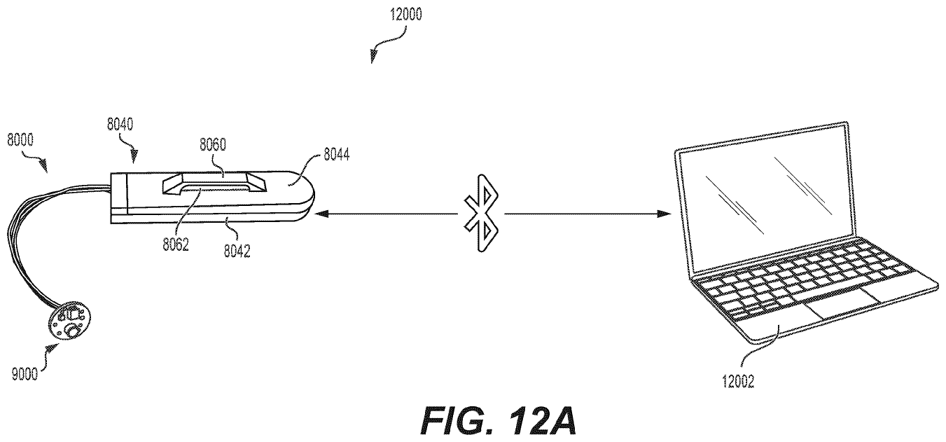

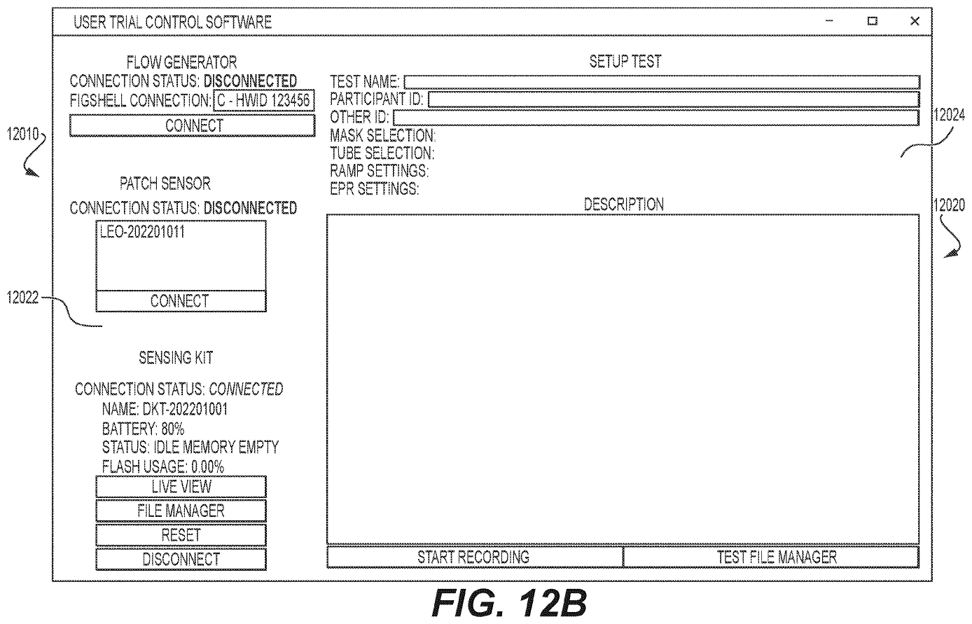

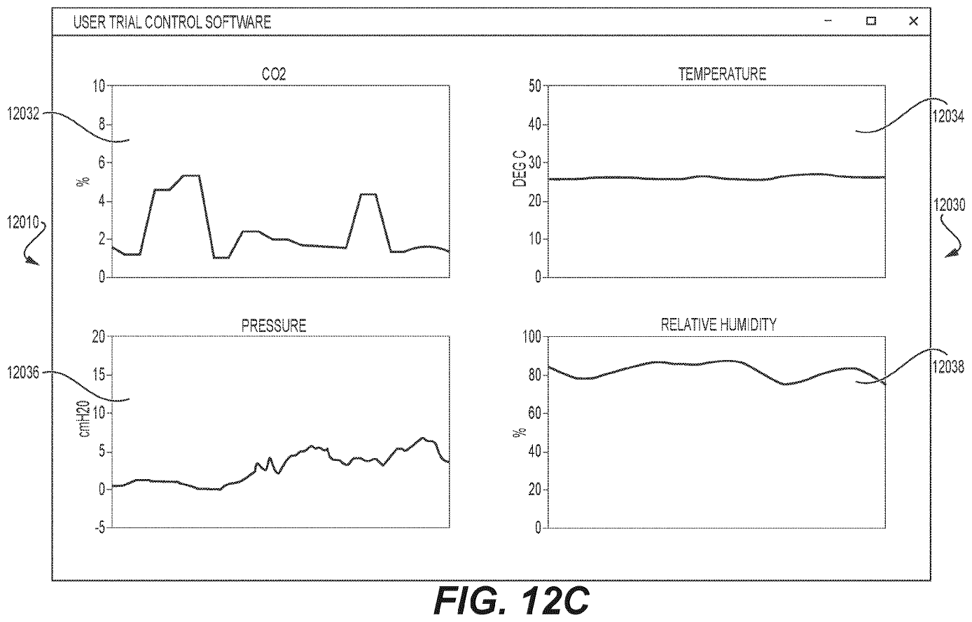

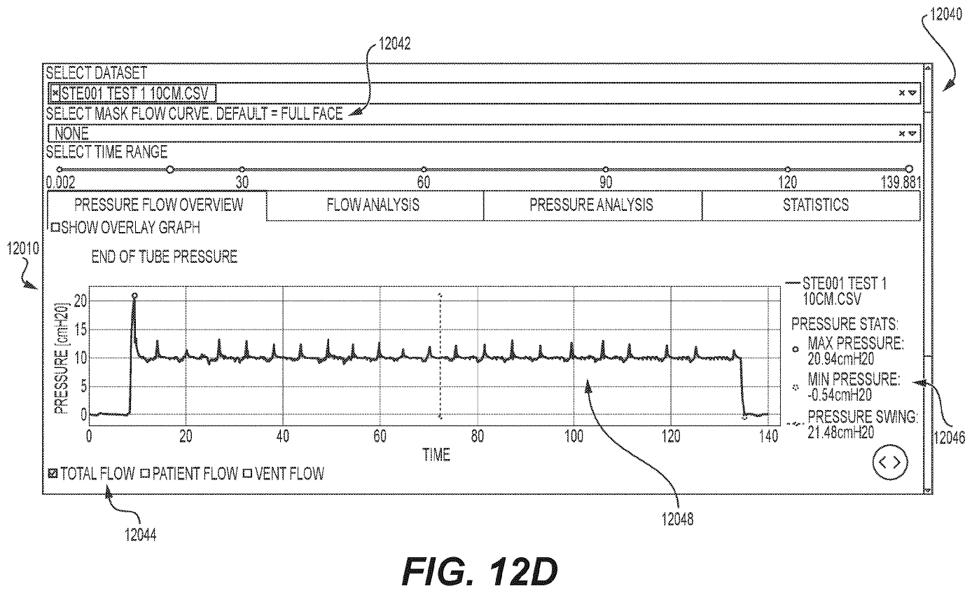

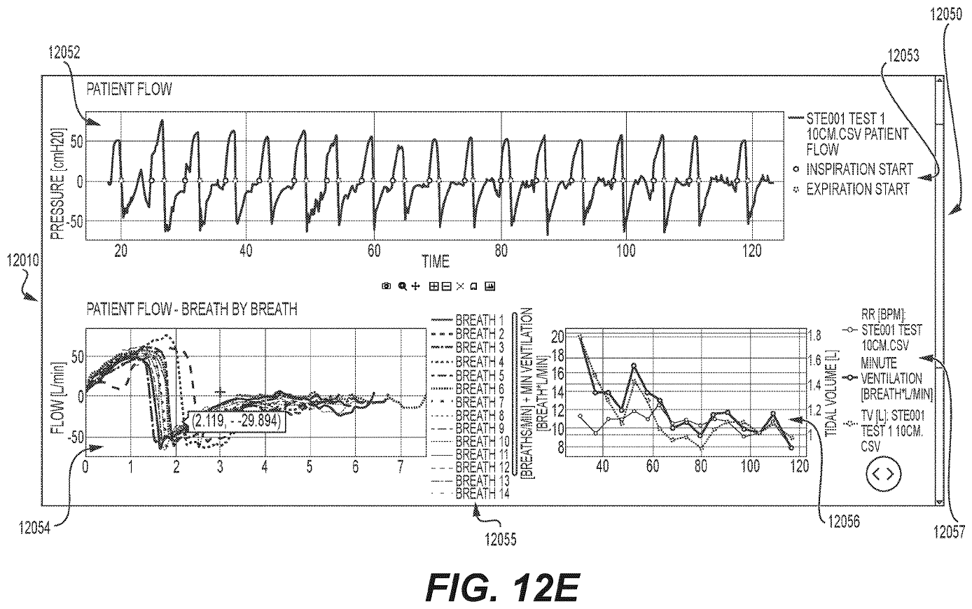

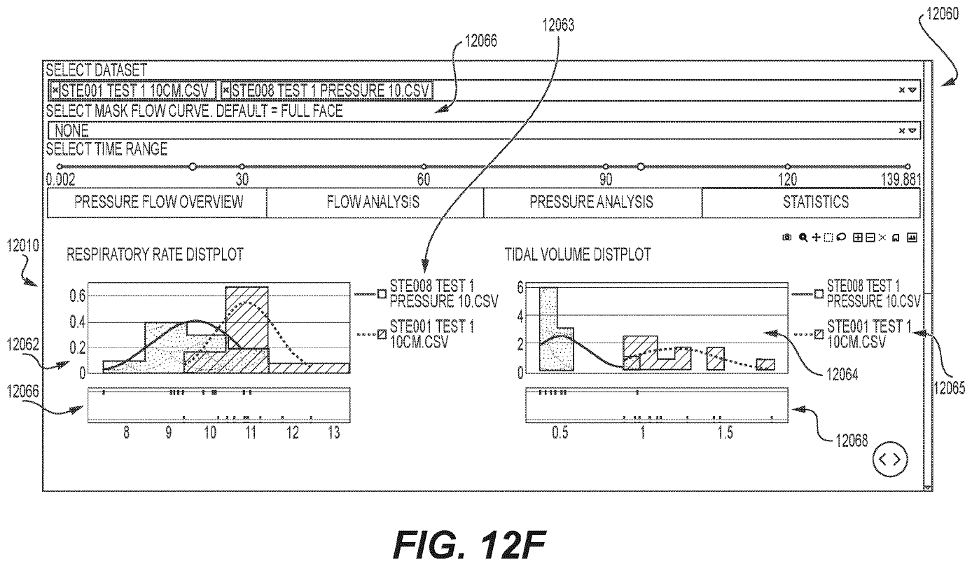

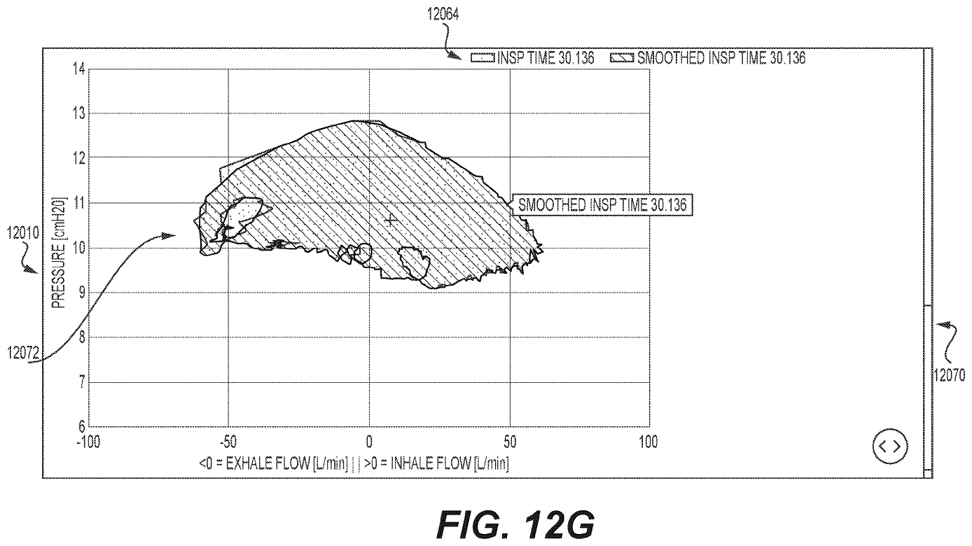

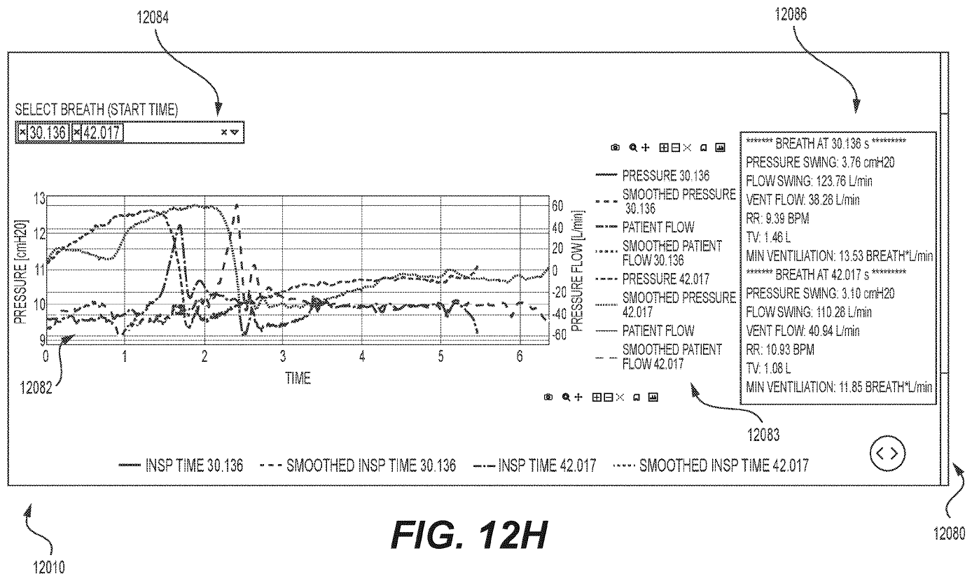

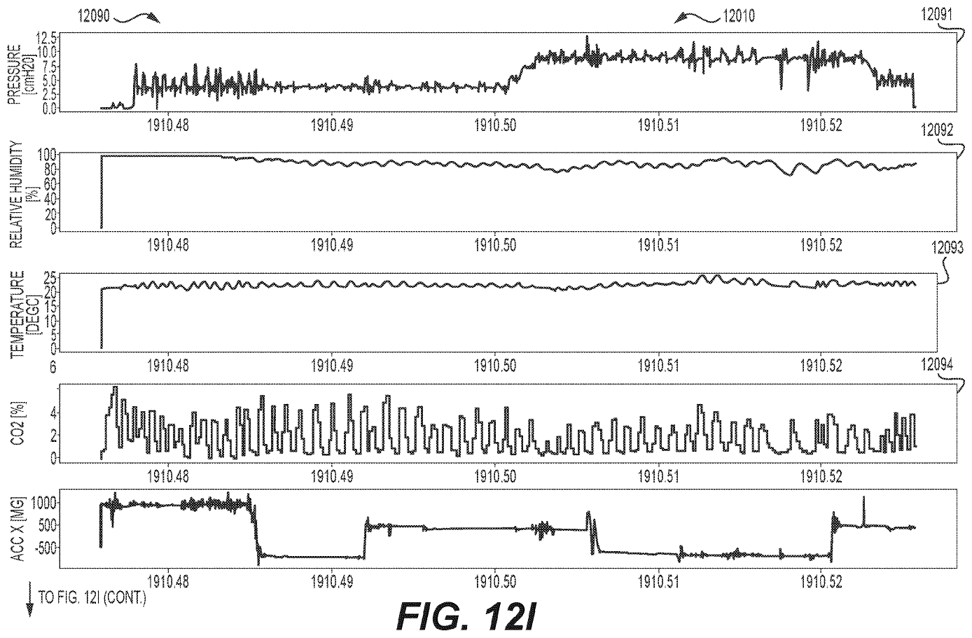

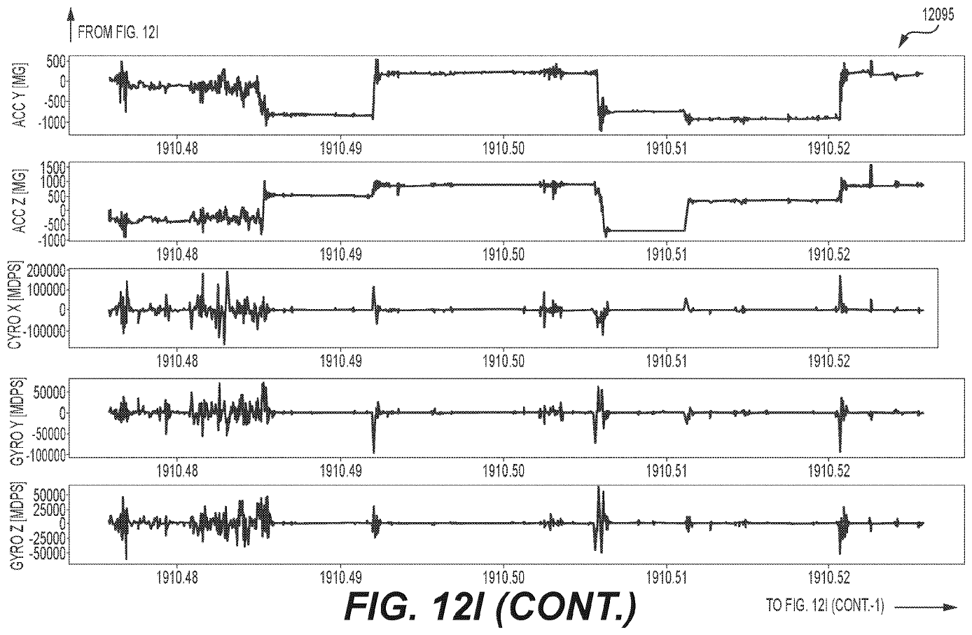

FIG. 12A depicts a system 12000 including an external device 12002 and the sensing kit 8000.

FIG. 12B is a screen 12020 of a user interface 12010 for interacting with devices and viewing device status.

FIG. 12C depicts a screen 12030 of the user interface 12010 for viewing data outputs from the sensing kit 8000.

FIG. 12D depicts a screen 12040 of the user interface 12010 for viewing data from one or more sources.

FIG. 12E depicts a screen 12050 of the user interface 12010 for viewing data from one or more sources.

FIG. 12F depicts a screen 12060 of the user interface 12010 for viewing data from one or more sources.

FIG. 12G depicts a screen 12070 of the user interface 12010 for viewing data from one or more sources.

FIG. 12H depicts a screen 12080 of the user interface 12010 for viewing data from one or more sources.

FIG. 12I depicts a screen 12090 of the user interface 12010 for viewing data from one or more sources.

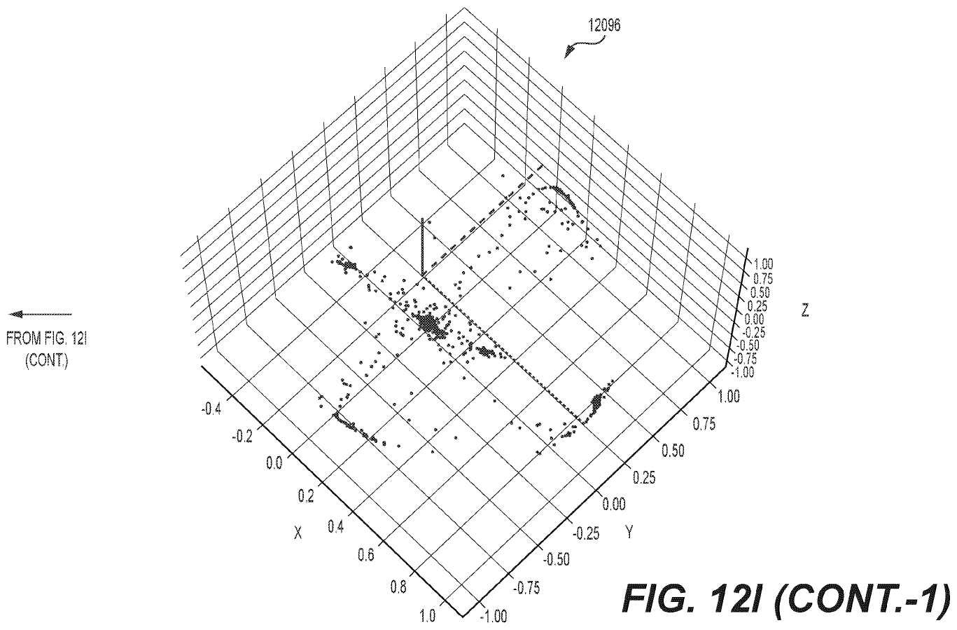

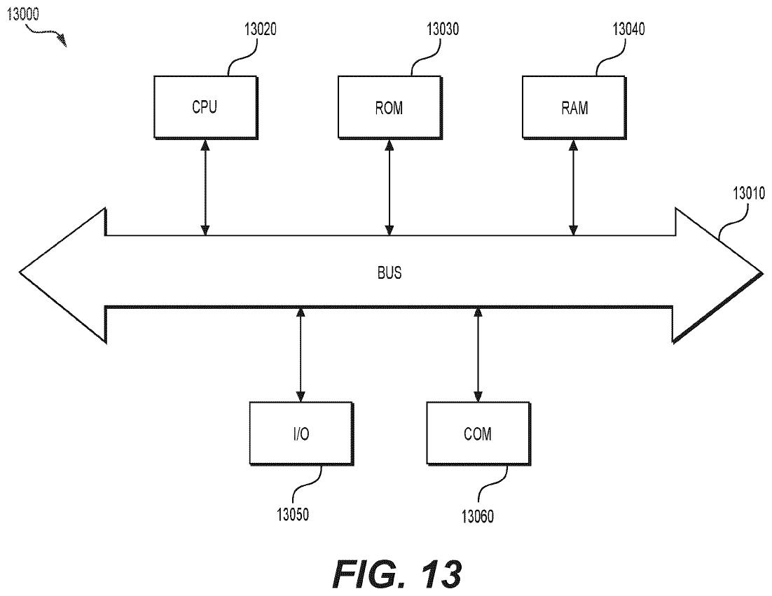

FIG. 13 depicts an example computer system 13000.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 THERAPY

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 RESPIRATORY THERAPY SYSTEMS

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 PATIENT INTERFACE

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

If a patient interface is unable to comfortably deliver a minimum level of positive pressure to the airways, the patient interface may be unsuitable for respiratory pressure therapy

The patient interface 3000 in accordance with one form of the present technology is constructed and arranged to be able to provide a supply of air at a positive pressure of at least 6 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 5.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.5 Forehead Region

In one form, the seal-forming structure that forms a seal in use on a forehead region of the patient's face. In such a form, the plenum chamber may cover the eyes in use.

### 5.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 5.3.3 Positioning and Stabilising Structure

The seal-forming structure 3100 of the patient interface 3000 of the present technology may be held in sealing position in use by the positioning and stabilising structure 3300.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

### 5.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 5.3.5 Decoupling Structure(s)

In one form the patient interface 3000 includes at least one decoupling structure, for example, a swivel or a ball and socket.

### 5.3.6 Connection Port

Connection port 3600 allows for connection to the air circuit 4170.

### 5.3.7 Forehead Support

In one form, the patient interface 3000 includes a forehead support 3700.

### 5.3.8 Anti-Asphyxia Valve

In one form, the patient interface 3000 includes an anti-asphyxia valve.

### 5.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.3.10 Sensing Kit

In some forms of the present technology, as shown in FIGS. 8A-11B, a sensing kit 8000 is provided. In some aspects, the sensing kit 8000 may include a patient interface 8010 and one or more sensors for measuring aspects of a device, such as the RPT device 4000, the patient interface 8010, a therapy administered via patient interface 8010, the patient's psychological or sleep data, and/or an environment near or at the patient. The sensing kit 8000 also may include one or more circuit boards for mounting sensors, transmitting signals, receiving signals, or processing signals. Although exemplary sensors are discussed herein with respect to the sensing kit 8000, the sensors described are non-limiting examples. The sensing kit 8000 may utilize any type of desired sensor, and sensors may be combined in various combinations.

The sensing kit 8000 may include sensors configured to, for example, evaluate breathing comfort of a patient. Sensors may include, but not be limited to, CO₂ sensor(s), pressure sensor(s), temperature sensor(s), humidity sensor(s), and/or accelerometer(s). Parameters measured by the sensors may be correlated with subjective reports of a patient's breathing comfort. For example, if a patient reports stuffiness while using the RPT device 4000 and the patient interface 8010, then the sensors of the sensing kit 8000 may measure values for CO₂ level, temperature, and/or humidity that correspond to stuffy conditions. In another example, if a patent reports that breathing feels out of sync, then the sensors may measure value(s) for pressure, and the RPT device 4000 may reflect values for flow generation that correspond to out of sync breathing. Various types of patient feedback regarding breathing comfort may be correlated with various sensor outputs/measurements. The sensor reporting system 12000, discussed below, may provide information regarding the sensor outputs/measurements, as well as other information.

A user may use the patient's feedback (e.g., the patient's subjective feedback) along with the measurements from the sensors in order to determine ideal or satisfactory operating parameters for the RPT device 4000 and/or the patient interface 8010. Such ideal or satisfactory operating parameters may be individualized for a particular patient or may apply to populations or sub-populations of patients. As discussed in further detail below, in an example of the present disclosure, the data obtained from the sensing kit 8000 may be used to calibrate the RPT device 4000 and/or the patient interface 8010 for a patient. For example, during setup of the RPT device 4000 and/or the patient interface 8010 (or during use of the RPT device 4000 and/or the patient interface 8010), the patient may report a subjective or objective comfort or discomfort level or another assessment. In examples, the patient may make such reports via an app on a computer or mobile device, via an interface (e.g., a button, touchscreen, knob, switch, or other actuator) of the RPT device 4000 or the patient interface 8010, via a remote device, orally to an automated system or to personnel, or via other mechanisms. Based on the patient's reporting/feedback, the RPT device 4000 and/or the patient interface 8010 may be automatically or manually adjusted. For example, a flow rate, flow pressure, flow volume, flow timing, temperature, venting, humidity, and/or other aspect may be adjusted.

In another example, reporting by a patient (e.g., according to the mechanisms described above) may be utilized to calibrate and/or adjust the RPT device 4000 or the patient interface 8010 of other patients. For example, if one patient reports discomfort or increased comfort, information obtained from sensors of the sensing kit 8000 for that patient may be used in order to optimize the RPT devices 4000 or patient interfaces 8010 of other patients. Adjustments may be made automatically or manually to the other patients' RPT devices 4000 or patient interfaces 8010 (e.g., through a software update), and/or the patients (or their healthcare providers) may be instructed (e.g., automatically instructed) regarding adjustments to make to their RPT devices 4000 or patient interfaces 8010. Additionally or alternatively, an alternative RPT device or patient interface may be recommended to the patient or a patient's healthcare provider. In a further example, measurements made by the sensing kit 8000 may be used to develop new RPT devices 4000 and/or patient interfaces 8010 that create ideal or satisfactory breathing comfort. It may be determined that certain properties of the RPT devices 4000 and/or the patient interfaces 8010 may be resulting in sensor measurements from the sensing kit 8000 that correspond to high or low levels of patient comfort, and new devices and/or patient interfaces may be developed in order to increase or retain patient comfort.

All or portions of the sensing kit 8000 may optionally be coupled to the patient interface 8010. The patient interface 8010 may be similar to the patient interface 3000 described above (see e.g. FIG. 3A), and only some similarities and differences may be described. The patient interface 8010 is merely exemplary, and other types of patient interfaces (e.g., the patient interface 3800) may be utilized with appropriate modifications to the sensing kit 8000 and/or patient interface. The patient interface 8010 may include a plenum chamber 8012 and a seal-forming structure 8014. The plenum chamber 8012 may have any of the properties of the plenum chamber 3200, and the seal-forming structure 8014 may have any of the properties of the seal-forming structure 3100. The patient interface 8010 may include a positioning and stabilising structure 8020 having at least one strap. The positioning and stabilizing structure 8020 may have any of the properties of the positioning and stabilizing structure 3300. As shown in FIG. 8A, the positioning and stabilising structure 8020 may include a pair of first, upper straps 8022 and a pair of second, lower straps 8024. Although only one of each pair of straps 8022, 8024 is depicted, it will be appreciated that straps 8022, 8024 may be symmetrical on either side of the plenum chamber 8012. The positioning and stabilizing structure 8020 may include additional straps or fewer straps and may have various configurations.

The sensing kit 8000 may include a first circuit board assembly 8030, a second circuit board assembly 9000, and a connector 9050 connecting the first circuit board assembly 8030 and the second circuit board assembly 9000. In aspects of the present disclosure, the first circuit board assembly 8030 and the second circuit board assembly 9000 may be coupled (either fixedly or removably) to the patient interface 8010. As depicted in FIG. 8A, the first circuit board assembly 8030 may be removably affixed to the positioning and stabilizing structure 8020. For example, the first circuit board assembly 8030 may be coupled to the first strap 8022, outside of the plenum chamber 8012. Features of the first circuit board assembly 8030 that facilitate such coupling are described below. In alternatives, the first circuit board assembly 8030 may be attached to another portion of the positioning and stabilizing structure 8020 (e.g., to the second strap 8024). In further alternatives, aspects of the sensing kit 8000, such as the first circuit board assembly 8030 or the second circuit board assembly 9000, may be located on other types of patient interfaces, to the RPT device 4000, in a room where the patient is located (e.g., a bedroom), or elsewhere.

### 5.3.10.1 First Circuit Board Assembly

With reference to FIGS. 8B and 8C, the first circuit board assembly 8030 may include a first circuit board 8032. The first circuit board 8032 may have any suitable size and shape, and may be flexible or rigid. In some examples, the first circuit board 8032 may be approximately 60 mm by approximately 20 mm. The circuit board 8032 may have a shape that is approximately rectangular with a rounded end. For example, the first circuit board 8032 may include a rectangular-shaped portion with a rounded (e.g., approximately semicircular) portion extending from one side (e.g., a shorter side) of the rectangular-shaped portion, such that the first circuit board 8032 may include three substantially straight sides and one curved side. However, such dimensions and shape are merely exemplary, and the first circuit board 8032 may have any suitable size and shape. Although one first circuit board 8032 is depicted, it will be appreciated that multiple circuit boards may be used to house components of the first circuit board 8032.

The first circuit board 8032 may function as, for example, a motherboard. The first circuit board 8032 may include the logic for operating sensing kit 8000. The first circuit board may receive data from sensors of the second circuit board assembly 9000 and may control the sensors of the second circuit board assembly 9000. For example, the first circuit board 8032 may include (e.g., may have mounted thereon), a control element, such as a microcontroller (or microprocessor, integrated circuit, or the like), a charging circuit, communication modules (e.g., Bluetooth module, universal serial bus (“USB”) communications, connectors/interfaces for connecting to second circuit board assembly 9000, or modules for connecting to WiFi or other networks). The first circuit board 8032 may include memory mounted thereon, either as a component of the microcontroller or as a separate element. The first circuit board 8032 also may include sensors, such as an accelerometer, electroencephalography (“EEG”) sensor(s), or other types of sensor(s). The first circuit board 8032 may store data gathered from sensors of the sensing kit 8000. The first circuit board assembly 8030 also may include a battery 8034. The battery 8034 may be separate from (and may be connected to) the first circuit board 8032, as shown, or may be mounted on the first circuit board 8032. Wires or cables 8038 may extend between the battery 8034 and the first circuit board 8032. Elements of the first circuit board 8032 may be arranged in any suitable manner. Furthermore, first circuit board 8032 may include elements additional to those exemplary elements discussed herein.

The first circuit board assembly 8030 may include a housing 8040, details of which are depicted in FIGS. 8D-8F. The housing 8040 may include features configured to receive and retain the first circuit board 8032. The housing 8040 may have a shape that is approximately complementary to a shape of the first circuit board 8032 (e.g., three substantially straight sides and one curved side). The housing 8040 may include a first piece 8042 and a second piece 8044. When assembled together, the first piece 8042 and the second piece 8044 may together form the housing 8040. Each of the first piece 8042 and the second piece 8044 may have a shape that is approximately complementary to a shape of the first circuit board 8032. The housing 8040 may include any suitable material (e.g., plastic or metal) and may be manufactured using any suitable method (e.g., three-dimensional (“3D”) printing).

The first piece 8042 may receive the first circuit board 8032. The first piece 8042 may include a substantially flat surface 8046 and a rim 8048 about a perimeter of the first piece 8042. A retaining strap 8050 may extend along one side of the first piece 8042 (e.g., a straight side of the first piece opposite a curved side of the first piece 8042). The strap 8050 may extend approximately parallel to the substantially flat surface 8046, with a channel being defined between the substantially flat surface 8046 and the strap 8050. The first piece 8042, including substantially flat surface 8046, rim 8048, and strap 8050, may be formed from a single, monolithic piece of material. The first circuit board 8032 may be positioned in/on the first piece 8042, such that an end of the first circuit board 8032 (e.g., a flat end of the first circuit board 8032 opposite a curved end of the first circuit board 8032) is received within the channel defined by the strap 8050 and the substantially flat surface 8046. The strap 8050 may include a retaining feature (e.g., a protrusion) for retaining the first circuit board 8032 and fixing the first circuit board 8032 relative to the housing 8040. The strap 8050 and/or the housing 8040 may define one or more openings through which the connector 9050 may pass.

The second piece 8044 of the housing 8040 may be a lid for the first piece 8042 and may have a shape approximately complementary to a shape of the first piece 8042 (e.g., the second piece 8044 may include three straight sides and one curved side). The second piece 8044 may, in some forms of housing 8040, receive the battery 8034. The second piece 8044 may include a flat portion 8052 and straps 8054. For example, as depicted, the second piece 8044 may include two straps 8054. Alternatively, the second piece 8044 may include alternative numbers of straps 8054 or may lack straps 8054. The battery 8034 may be received between the flat portion 8052 and the straps 8054 to restrain the battery 8034.

The second piece 8044 also may include one or more retaining features 8056. As shown, the second piece 8044 may include two retaining features 8056, which may be protrusions. A first retaining feature 8056 may extend from or near one of straps 8054 (e.g., from or near a strap 8054 closer to a straight side of the second piece 8044 that is opposite to the curved side of the second piece 8044). A second retaining feature 8056 may extend from a side of the second piece 8044 (e.g., from the curved side of second piece 8044). The retaining features 8056 may engage with features of first piece 8042 in order to secure the second piece 8044 to the first piece 8042. For example, the first retaining feature 8056 may engage with the strap 8050 of the first piece 8042. The second retaining feature 8056 may engage with the rim 8048 of the first piece 8042. For example, the rim 8048 may include a notch 8058 or other feature for engaging with the retaining feature 8056. As depicted, the notch 8050 may be present on the first piece 8042. In another example, the notch 8058 may be present on the second piece 8044 while the opposing retaining feature 8056 may be present on the first piece 8042.

An outer surface of the housing 8040 (e.g., an outer surface of the second piece 8044) may include a frame 8060. The frame 8060 may extend outward from the second piece 8044 and may define an opening 8062. The frame 8060 may be sized and shaped so that a securing strap 8064 may be received by the opening 8062. The securing strap 8064 may include, for example, hook-and-loop fastening material or another type of fastener (e.g., button, snap, rivet, buckle). The securing strap 8064 may be affixed to, for example, the positioning and stabilizing structure 8020. As shown, the securing strap 8064 may be wrapped around and affixed to the first strap 8022. Alternatively, the securing strap 8064 may be affixed to another portion of the positioning and stabilizing structure 8020, such as the second strap 8024, a tube, or another portion of patient interface 8010 or RPT device 4000.

### 5.3.10.2 Docking Station for First Circuit Board Assembly

As shown in FIGS. 8G-8J, the sensing kit 8000 also may include a docking station 8070 for docking with and transferring information to and/or from the first circuit board assembly 8030. The docking station 8070 may include a docking board 8072 and a housing 8074. The docking board 8072 may include one or more circuit boards that may include circuitry for performing various functions. For example, the docking board 8072 may include a charging circuit, a power source, an external communication circuit (e.g., via USB), and one or more lights (e.g., LEDs).

As best shown in FIGS. 8E, the first piece 8042 of the housing 8040 may include a plurality of openings 8066 formed therein. For example, the housing 8040 may include four openings 8066. The openings 8066 may be configured to allow passage of pogo pins 8076 of the docking board 8072 to pass therethrough in order to form an electrical communication with the first circuit board 8032. A number of pogo pins 8076 may correspond to a number of openings of the housing 8040 and an interface of the first circuit board 8032. While the first circuit board assembly 8030 is docked to the docking station 8070, a variety of functions may be performed. For example, a charging circuit of the docking station 8070 may cooperate with circuitry of the first circuit board 8032 (e.g., charging circuitry of the first circuit board 8032) to charge the battery 8034, or the charging circuit of the docking station 8070 may directly charge the battery 8034. Charging may occur via an external power source or an on-board power source of the docking station 8070. Additionally or alternatively, information (e.g., sensor data, settings, updates, etc.) may be transferred between the first circuit board 8032 and the docking board 8072. When the first circuit board assembly 8030 is docked to the docking station 8070, the lights of the docking board 8072 may illuminate to reflect the connection. In an example, one or more lights may illuminate a first color when the battery 8034 is charging and one or more lights may illuminate a second color when the battery 8036 is fully charged.

The docking board 8072 may include circuitry for communicating with external devices (e.g., computers, tablets, phones, other mobile devices, etc., either directly or via a network). For example, the docking board 8072 may include USB circuitry, Bluetooth circuitry, and/or circuitry for connecting to wired or wireless networks. Via such circuitry, the docking board 8072 may share information (e.g., information obtained from first circuit board 8032) with other devices.

The docking board 8072 may be received within the housing 8074. The housing 8074 may serve to secure the first circuit assembly 8030 with respect to the docking board 8072 to ensure connection between the first circuit board 8032 and the docking board 8072. Elements of housing 8074 may be formed of any suitable material (e.g., plastic or metal) and formed according to any suitable manufacturing method (e.g., 3D printing). The housing 8074 may include a cradle 8078, which may define a recessed portion 8080 having a complementary shape to portions of the first circuit assembly 8030 (e.g., a complementary shape to the straight sides of the housing 8040 extending between the opposing curved and straight sides of the housing 8040). The recessed portion 8080 may be configured to receive the first circuit assembly 8030 when the first circuit assembly 8030 is docked to the docking station 8070. The cradle 8078 may define one or more openings 8082. Although three openings 8082 are depicted, any suitable number of openings 8082 may be used. The pogo pins 8076 may extend through one of the openings 8082, in a position configured to align with the openings 8066 of the housing 8040 of the first circuit board assembly 8030. As shown in FIG. 8J, an end portion 8084 of the recessed portion 8080 may extend beyond an end of the first circuit board assembly 8030 to allow a user to grip and position/remove the first circuit assembly 8030 from the docking station 8070.

The housing 8074 also may include a base 8086. The cradle 8078 may be coupled to the base 8086, such that the cradle 8078 and the base 8086 extend approximately parallel to one another. A slot 8088 may extend between the cradle 8078 and the base 8086 for receiving the docking board 8072. The docking board 8072 may be coupled to the housing 8074 via, for example, mating features, adhesive, friction fit, or other mechanisms. The housing 8074 may further include a clamp 8090. The clamp 8090 may be slidably coupled to the housing 8074. The clamp 8090 may include, for example two legs 8092 and a crossbar 8094 extending therebetween. Each leg 8092 may include a protrusion 8096 extending inwardly, toward the other of the legs 8092. As shown particularly in FIG. 8J, sides of the cradle 8078 may define slots 8098 for receiving the protrusions 8096. The protrusions 8096 may be slidably received within the slots 8098, such that the clamp 8090 may move along the cradle 8078. When the first circuit assembly 8030 is placed within the cradle 8078, the clamp 8090 may be slid over the first circuit assembly 8030 to securely hold the first circuit assembly 8030 within the housing 8074 to ensure a robust, reliable connection between the first circuit assembly 8030 and the docking station 8070.

### 5.3.10.3 Second Circuit Board Assembly

In some forms, the sensing kit 8000 also may include the second circuit board assembly 9000, depicted in FIGS. 9A-9G. The second circuit board assembly 9000 may be coupled to the first circuit board assembly 8030 via the connector 9050, discussed below. The second circuit board assembly 9000 may be coupled to, for example, the patient interface 8010. For example, the second circuit board assembly 9000 may be coupled to a frame 8016 of the plenum chamber 8012 and/or the seal-forming structure 8014. Alternatively, the second circuit board assembly 9000 may be coupled to other types of patient interfaces, to the RPT device 4000, in a room where the patient is located (e.g., a bedroom), or elsewhere.

The second circuit board assembly 9000 may include one or more sensors, including, but not limited to any combination of a CO₂ sensor, a pressure sensor, a temperature sensor, a humidity sensor, an accelerometer, a flow rate sensor, an infrared sensor, a photoplethysmogram (PPG) sensor, an electrocardiogram (ECG) sensor, an electroencephalography (EEG) sensor, a capacitive sensor, a force sensor, a strain gauge sensor, an electromyography (EMG) sensor, an oxygen sensor, an analyte sensor, a moisture sensor, a light detection and ranging (LiDAR) sensor, an electrooculography (EOG) sensor, a peripheral oxygen saturation (SpCk) sensor, or a galvanic skin response (GSR) sensor. Additionally or alternatively, the second circuit board assembly 9000 may include radiofrequency (“RF”) sensors, such as near field communications (“NFC”) sensors, for identification of the second circuit board assembly 9000 and/or the patient interface 8010. In one form, the second circuit board assembly 9000 may include a pressure sensor, a CO₂ sensor, and a combined temperature and humidity sensor. The sensor(s) of the second circuit board assembly 9000 may measure data pertinent to a patient's breathing comfort, among other data. Some sensors of the second circuit board assembly 9000 may require access to both an environment within the plenum chamber 8012 and an environment outside of the plenum chamber 8012. For example, a pressure sensor may require access to an environment outside of the plenum chamber 8012, as well as access to the plenum chamber 8012. Others of the sensors may require access only to an interior of the plenum chamber 8012 or only to an exterior of the plenum chamber 8012.

The second circuit board assembly 9000 include a second circuit board 9010. The second circuit board 9010 may be rigid or flexible (e.g., a rigid or flexible printed circuit board). In some forms, the second circuit board 9010 may be approximately circular. In alternatives, the second circuit board 9010 may have other shapes. For example, the second circuit board 9010 may be flexible and may have an approximately rectangular shape with sensors mounted in a row thereon. In configurations in which the second circuit board 9010 is flexible, it may conform to a shape of the plenum chamber 8012 (e.g., a complex shape defined by frame 8016 or seal-forming structure 8014). A width (e.g., a diameter) of the second circuit board 9010 may be approximately 7.0 mm to approximately 19 mm, approximately 10.0 mm to approximately 16 mm, or approximately 13 mm. Sensors may be mounted to the second circuit board 9010. For example, as depicted in FIGS. 9D and 9E, a pressure sensor 9012, a CO₂ sensor 9014, and a combined temperature and humidity sensor 9016 may be mounted to the second circuit board 9010. However, the dimension of the second circuit board 9010, an arrangement and choice of the sensors of the second circuit board 9010 are merely exemplary. Other sensor(s) may be utilized in any combination. In some examples, the sensors of the second circuit board 9010 may be modular, such that one or more sensors may be replaced with other sensor(s). Additionally or alternatively, the first circuit board 8010 may be utilized with various different second circuit boards 9010, having different combinations and/or arrangements of sensor(s). In some examples, an opening may be formed in the frame 8016 and/or the seal-forming structure 8014 for receiving the second circuit board assembly 9000. Such an opening may be formed using a drill, a punch (e.g. a 10 mm drill or punch or another size drill or punch that is slightly smaller than a diameter of the grommet 9020) or via an alternative method. In one form, the opening may extend through the frame 8016 and a flexible seal-forming structure 8014 (e.g., a cushion), such as a liquid silicone rubber (“LSR”) cushion. In such a form, as shown in FIGS. 9A-9C, a grommet 9020, which may be relatively rigid, may hold the second circuit board 9010. The grommet 9020 may include, for example, plastic or metal and may be formed by, for example 3D printing. In some forms, the grommet 9020 may have an approximately circular cross-section, sized so as to receive the second circuit board 9010 and to be received within the opening of the frame 8016 and seal-forming structure 8014.

In an alternative, a grommet or other structure to securely receive the second circuit board 9010, may be integrally formed with any suitable portion of patient interface 8010, including for example, the frame 8016 and/or the seal-forming structure 8014. In this form, a subsequent manufacturing step to form an opening to receive the second circuit board assembly 9000 is not needed.

As shown in FIG. 9B, the grommet 9020 may have an approximately spool shape, with a first flange 9022 at a first end of grommet 9020 and a second flange 9024 at a second end of grommet 9020. A central lumen 9026 may extend through the first flange 9022, the second flange 9024, and a spool-like portion of grommet 9020 extending therebetween. The first flange 9022 may include a plurality of protrusions 9028 extending therefrom, in a direction away from the second flange 9024. The protrusions 9028 may define a seat for the second circuit board 9010, and hold the second circuit board firmly onto the first flange 9022. The protrusions 9028 may contain small extensions that clamp the second circuit board onto the gasket 9030 located on the first flange 9022.

The second circuit board assembly 9000 also may include a gasket 9030, which may be formed from, for example, silicone. The gasket 9030 may be flexible and may be shaped similarly to a washer. When the second circuit board assembly 9000 is assembled, the gasket 9030 may be positioned on the first flange 9022, surrounded by the protrusions 9028. The second circuit board 9010 may be positioned on the gasket 9030. Thus, the gasket 9030 may be between the second circuit board 9010 and the first flange 9022. As shown in FIG. 9A, the protrusions 9028 may be configured to exert a force on the second circuit board 9010 and/or the gasket 9030, to retain the second circuit board 9010 relative to the grommet 9020. For example, the protrusions 9028 may include portions extending radially inward toward the lumen 9026, which may exert forces on the second circuit board 9010 in a direction toward the first flange 9022. In some forms, the gasket 9030 may be compressed when the circuit board assembly 9000 is assembled. Alternatively or additionally, the second circuit board 9010 may be coupled to the grommet 9020 via adhesive or via other coupling components (not shown).

As shown in FIG. 9C, the grommet 9020 may be installed on the frame 8016 and the seal-forming structure 8014. When disposed in the opening of the plenum chamber 8012, the grommet 9020 may form a seal with the plenum chamber 8012. For example, materials of the grommet 9020, the frame 8016, and the seal-forming structure 8014 may facilitate the forming of such a seal between the grommet 9020 and the plenum chamber 9012. The first flange 9022 may be disposed on an inner surface of the seal-forming structure 8014, facing an interior of the plenum chamber 8012. The second flange 9024 may be disposed on an outer surface of frame 8016. Materials of the grommet 9020, the frame 8016 and the seal-forming structure 8014 may be such that a seal forms about the grommet 9020. In some forms, the grommet 9020 is retained within the frame 8016 and the seal-forming structure 8014 without adhesive, for example, via a friction fit. In alternatives, adhesive may be utilized to retain the grommet 9020. In some forms, a surface of the second circuit board 9010 may be exposed within an interior of plenum chamber 8012. Alternatively, the second circuit board 9010 may be covered (e.g., overmolded) or potted. In configurations in which the second circuit board 9010 is covered/potted, some or all of the sensors of circuit board 9010 (e.g., a pressure sensor or a CO₂ sensor) may be exposed as necessary for operation of the sensor(s). One or more sensors 9032 of the second circuit board 9010 may be disposed on a portion of second circuit board 9010 facing the lumen 9026 of the grommet 9020, such that the sensor 9032 is exposed to the atmosphere outside of the plenum chamber 8012. For example, the sensor 9032 may be partially disposed within the lumen 9026, and the lumen 9026 may be in communication with the atmosphere outside of the plenum chamber 9012. For example, the sensor 9032 may include a pressure sensor that includes a portion that is exposed to the interior of the plenum chamber 8012 and a portion that is exposed to the atmosphere outside of the plenum chamber 8012.

In an alternative, an opening may extend through the frame 8016 and/or a rigid (e.g., polycarbonate) seal-forming structure 8014, as shown in FIG. 9F. In such an example configuration, the second circuit board assembly 9000 may include a grommet 9040, shown in FIG. 9G, that is relatively flexible. The grommet 9040 may be formed (e.g., molded) from a material such as LSR. The grommet 9040 may include any of the structures of grommet 9020, discussed above. For example, the grommet 9040 may include a first flange 9042 and a second flange 9044. The first flange 9042 may include a rim 9046 extending therefrom for securing the second circuit board 9010 to the first flange 9042. A lumen 9048 may extend through the first flange 9042, the second flange 9044, and a spool-like portion of the grommet 9040 extending therebetween. As with the grommet 9020, the grommet 9040 may be positioned such that an outer surface of the first flange 9042 faces an interior of the plenum chamber 8012, and an outer surface of the second flange 9044 faces the environment external to the plenum chamber 8012. The grommet 9040 may form a seal with the frame 8016 and/or the seal-forming structure 8014 when disposed in the opening of plenum chamber 8012.

In another form, as shown in FIG. 10, an alternative patient interface 10000 (having any of the properties of the patient interfaces 3000, 8010) may include a plenum chamber 10012 (having any of the properties of the plenum chambers 8012, 3200), a seal-forming structure 10014 (having any of the properties of the seal-forming structures 3100, 8014) and a frame 10016 (having any of the properties of the frame 8016). The frame 10016 may include a vent 10018, having any of the features of the vent 3400. As shown, the vent 10018 may include a plurality of openings between plenum chamber 10012 and an external atmosphere.

A second circuit board assembly 10020 (which may be a component of the sensing kit 8000) may be at least partially mounted on an external surface of plenum chamber 10012/frame 10016. For example, a second circuit board 10022 (having any of the properties of second circuit board 9010) may be adhered to a front of patient interface 10000. An outer surface of the second circuit board 10022 may be encapsulated and sealed. One or more sensors may be disposed/mounted within plenum chamber 10012. The sensors may include, for example, a pressure sensor, a CO₂ sensor, and/or a humidity sensor. A wire/cable 10024 may extend from the sensor(s) to the second circuit board 10022, through an opening of the vent 10018, providing electrical connections (e.g., signal communications and/or power) between the second circuit board 10022 and the sensors. The second circuit board assembly 10020 may be equipped to communicate wirelessly with the first circuit board assembly 8030 or may include the functionality of the first circuit board assembly 8030. For example, the second circuit board assembly 10020 may include a microprocessor or microcontroller with wireless capabilities. The second circuit board assembly 10020 also may include connections for charging a battery of the second circuit board assembly 10020.

In another form, as shown in FIGS. 11A-11B, an alternative patient interface 11000 (having any of the properties of patient interfaces 3000, 8010, 10000) may include a plenum chamber 11012 (having any of the properties of plenum chambers 3200, 8012, 10012), a seal-forming structure 11014 (having any of the properties of seal-forming structures 3100, 8014, 10014) and a frame 11016 (having any of the properties of frames 8016, 10014). The frame 11016 may include a vent 11018, having any of the features of vents 3400, 10018. As shown, vent 11018 may include a plurality of openings between plenum chamber 11012 and an external atmosphere.

A second circuit board assembly 11020 (which may be a component of the sensing kit 8000) may be at least partially mounted on an internal surface 11034 of the plenum chamber 11012/frame 11016. For example, a second circuit board 11022 (having any of the properties of the second circuit boards 9010, 10022) may be adhered to an internal surface of the frame 11016. At least portions of a surface of the second circuit board 11022 may optionally be encapsulated and sealed.

One or more sensors may be disposed/mounted on the second circuit board 11022, as discussed above with respect to the second circuit board 9010. The sensors may include, for example, a pressure sensor, a CO₂ sensor, and/or a humidity sensor. As shown in FIG. 11B, which depicts a cross-sectional view of the patient interface 11000 where the second circuit board 11022 is mounted to the frame 11016, the second circuit board 11022 may be affixed to the frame 11016 using adhesive 11024. A sensor 11026 (e.g., a pressure sensor) may be mounted to the second circuit board 11022. The second circuit board 11022 may include an opening 11028, which may be at least partially aligned with an opening 11030 of the vent 11018. Via the opening 11028 of the second circuit board 11022 and the opening 11030 of the vent 11018, the sensor 11026 may be able to access and measure an atmosphere/environment outside of plenum chamber 11012 (an area to the right of the frame 11016 in FIG. 11B). The sensor 11026 also may be able to access and measure an environment within plenum chamber 11012 (an area to the left of the frame 11016 in FIG. 11B).

The second circuit board assembly 11020 may be equipped to communicate wirelessly with the first circuit board assembly 8030 or may include the functionality of the first circuit board assembly 8030. For example, the second circuit board assembly 11020 may include a microprocessor or microcontroller with wireless capabilities. The second circuit board assembly 11020 also may include connections for charging a battery of the second circuit board assembly 11020. One or more barbs 11032 may extend from the second circuit board 11022, through the opening 11030 of the vent 11018, and to an external surface 11036 of the frame 11016 in order to further secure the second circuit board 11022 to the frame 11016.

### 5.3.10.4 Connector

A connector (e.g., wire, cable, etc.) 9050 may extend between the first circuit board assembly 8030 and the second circuit board assembly 9000 (or 10020, 11020, in some forms) to electrically connect the first circuit board assembly 8030 and the second circuit board assembly 9000. The connector 9050 may include, for example, four wires, which may be separate wires or may be bundled together in a cable. A number of wires is merely exemplary, and alternative numbers of wires may be used, depending on the components of the first circuit board assembly 8030 and/or the second circuit board assembly 9000, 10020, 11020. The first circuit board 8032 and the second circuit board 9010, 10022, 11022 each may include an interface for connecting to connector 9050. In some examples, the connector 9050 may include an I²C bus. The connector 9050 may be compatible with various sensors and may allow addition of any I²C-capable sensors to the second circuit board 9010, 10022, 11022. For example, an entirety of the second circuit board 9010, 10022, 11022 may be removed and replaced with a different circuit board, or sensors of the second circuit board 9010, 10022, 11022 may be removable and replaceable with alternative sensors. The connector 9050 may transmit control signals and/or power from first circuit board assembly 8030 to the second circuit board assembly 9010, 10022, 11022, and transmit data from the second circuit board assembly 9010, 10022, 11022 to the first circuit board assembly 8030.

A particular configuration (e.g., length, width, etc.) of connector 9050 may be chosen based on the configurations of the first circuit board assembly 8030 and the second circuit board assembly 9000, 10020, 11020. For example, a length of connector 9050 may depend upon locations of the first circuit board assembly 8030 and the second circuit board assembly 9000, 10020, 11020 on the patient interface 8010, 10000, 11000. A length may be sufficient so as to extend between these assemblies without having so much slack that the slack interferes with the patient or is prone to snagging and/or unplugging.

### 5.4 RPT DEVICE

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH2O, or at least 10 cmH2O, or at least 20 cmH2O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 5.4.1 RPT Device Mechanical & Pneumatic Components

An RPT device may comprise one or more of the following components in an integral unit. In an alternative form, one or more of the following components may be located as respective separate units.

### 5.4.1.1 Air Filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface 3000 or 3800.

### 5.4.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000 or 3800.

### 5.4.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller 4240.

In other forms, a pressure generator 4140 may be a piston-driven pump, a pressure regulator connected to a high pressure source (e.g. compressed air reservoir), or a bellows.

### 5.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800.

In one form, a signal from a transducer 4270 may be filtered, such as by low-pass, high-pass or band-pass filtering.

### 5.4.1.4.1 Flow Rate Sensor

A flow rate sensor 4274 in accordance with the present technology may be based on a differential pressure transducer, for example, an SDP600 Series differential pressure transducer from SENSIRION.

In one form, a signal generated by the flow rate sensor 4274 and representing a flow rate is received by the central controller 4230.

### 5.4.1.4.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 4272 and representing a pressure is received by the central controller 4230.

### 5.4.1.4.3 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.4.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.4.2 RPT Device Electrical Components

### 5.4.2.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.4.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.4.2.3 Central Controller

In one form of the present technology, the central controller 4230 is one or a plurality of processors suitable to control an RPT device 4000.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and the humidifier 5000.

The central controller 4230 may be configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300 which may be implemented with processor-control instructions, expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.2.4 Clock

The RPT device 4000 may include a clock 4232 that is connected to the central controller 4230.

### 5.4.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 5.4.2.6 Protection Circuits

The one or more protection circuits 4250 in accordance with the present technology may comprise an electrical protection circuit, a temperature and/or pressure safety circuit.

### 5.4.2.7 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally, or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.4.2.8 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 5.4.2.9 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.4.2.9.1 Display Driver

A display driver 4292 receives as an input the characters, symbols, or images intended for display on the display 4294, and converts them to commands that cause the display 4294 to display those characters, symbols, or images.

### 5.4.2.9.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 5.4.3 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.4.3.1 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the vent flow rate Qv, the respiratory flow rate Qr, and the leak flow rate Ql.

In various forms of the present technology, the pre-processing module 4310 comprises one or more of the following algorithms: interface pressure estimation 4312, vent flow rate estimation 4314, leak flow rate estimation 4316, and respiratory flow rate estimation 4318.

### 5.4.3.1.1 Interface Pressure Estimation

In one form of the present technology, an interface pressure estimation algorithm 4312 receives as inputs a signal from the pressure sensor 4272 indicative of the pressure in the pneumatic path proximal to an outlet of the pneumatic block (the device pressure Pd) and a signal from the flow rate sensor 4274 representative of the flow rate of the airflow leaving the RPT device 4000 (the device flow rate Qd). The device flow rate Qd, absent any supplementary gas 4180, may be used as the total flow rate Qt. The interface pressure algorithm 4312 estimates the pressure drop ΔP through the air circuit 4170. The dependence of the pressure drop ΔP on the total flow rate Qt may be modelled for the particular air circuit 4170 by a pressure drop characteristic ΔP(Q). The interface pressure estimation algorithm, 4312 then provides as an output an estimated pressure, Pm, in the patient interface 3000 or 3800. The pressure, Pm, in the patient interface 3000 or 3800 may be estimated as the device pressure Pd minus the air circuit pressure drop ΔP.

### 5.4.3.1.2 Vent Flow Rate Estimation

In one form of the present technology, a vent flow rate estimation algorithm 4314 receives as an input an estimated pressure, Pm, in the patient interface 3000 or 3800 from the interface pressure estimation algorithm 4312 and estimates a vent flow rate of air, Qv, from a vent 3400 in a patient interface 3000 or 3800. The dependence of the vent flow rate Qv on the interface pressure Pm for the particular vent 3400 in use may be modelled by a vent characteristic Qv(Pm).

### 5.4.3.1.3 Leak Flow Rate Estimation

In one form of the present technology, a leak flow rate estimation algorithm 4316 receives as an input a total flow rate, Qt, and a vent flow rate Qv, and provides as an output an estimate of the leak flow rate Ql. In one form, the leak flow rate estimation algorithm estimates the leak flow rate Ql by calculating an average of the difference between total flow rate Qt and vent flow rate Qv over a period sufficiently long to include several breathing cycles, e.g. about 10 seconds.

In one form, the leak flow rate estimation algorithm 4316 receives as an input a total flow rate Qt, a vent flow rate Qv, and an estimated pressure, Pm, in the patient interface 3000 or 3800, and provides as an output a leak flow rate Ql, by calculating a leak conductance, and determining a leak flow rate Ql to be a function of leak conductance and pressure, Pm. Leak conductance is calculated as the quotient of low pass filtered non-vent flow rate equal to the difference between total flow rate Qt and vent flow rate Qv, and low pass filtered square root of pressure Pm, where the low pass filter time constant has a value sufficiently long to include several breathing cycles, e.g. about 10 seconds. The leak flow rate Ql may be estimated as the product of leak conductance and a function of pressure, Pm.

### 5.4.3.1.4 Respiratory Flow Rate Estimation

In one form of the present technology, a respiratory flow rate estimation algorithm 4318 receives as an input a total flow rate, Qt, a vent flow rate, Qv, and a leak flow rate, Ql, and estimates a respiratory flow rate of air, Or, to the patient, by subtracting the vent flow rate Qv and the leak flow rate Ql from the total flow rate Qt.

### 5.4.3.2 Therapy Engine Module

In one form of the present technology, a therapy engine module 4320 receives as inputs one or more of a pressure, Pm, in a patient interface 3000 or 3800, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea/hypopnea determination 4325, snore determination 4326, airway patency determination 4327, target ventilation determination 4328, and therapy parameter determination 4329.

### 5.4.3.2.1 Phase Determination

In one form of the present technology, the RPT device 4000 does not determine phase.

In one form of the present technology, a phase determination algorithm 4321 receives as an input a signal indicative of respiratory flow rate, Or, and provides as an output a phase Φ of a current breathing cycle of a patient 1000.

In some forms, known as discrete phase determination, the phase output Φ is a discrete variable. One implementation of discrete phase determination provides a bi-valued phase output Φ with values of either inhalation or exhalation, for example represented as values of 0 and 0.5 revolutions respectively, upon detecting the start of spontaneous inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output Φ is determined to have a discrete value of 0 (thereby “triggering” the RPT device 4000) when the respiratory flow rate Qr has a value that exceeds a positive threshold, and a discrete value of 0.5 revolutions (thereby “cycling” the RPT device 4000) when a respiratory flow rate Qr has a value that is more negative than a negative threshold. The inhalation time Ti and the exhalation time Te may be estimated as typical values over many respiratory cycles of the time spent with phase Φ equal to 0 (indicating inspiration) and 0.5 (indicating expiration) respectively.

Another implementation of discrete phase determination provides a tri-valued phase output Φ with a value of one of inhalation, mid-inspiratory pause, and exhalation.

In other forms, known as continuous phase determination, the phase output Φ is a continuous variable, for example varying from 0 to 1 revolutions, or 0 to 2π radians. RPT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:
1. If Qr is zero and increasing fast then Φ is 0 revolutions.
2. If Qr is large positive and steady then Φ is 0.25 revolutions.
3. If Qr is zero and falling fast, then Φ is 0.5 revolutions.
4. If Or is large negative and steady then Φ is 0.75 revolutions.
5. If Qr is zero and steady and the 5-second low-pass filtered absolute value of Qr is large then Φ is 0.9 revolutions.
6. If Qr is positive and the phase is expiratory, then Φ is 0 revolutions.
7. If Qr is negative and the phase is inspiratory, then Φ is 0.5 revolutions.
8. If the 5-second low-pass filtered absolute value of Qr is large, Φ is increasing at a steady rate equal to the patient's breathing rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the phase Φ is first discretely estimated from the respiratory flow rate Qr as described above, as are the inhalation time Ti and the exhalation time Te. The continuous phase Φ at any instant may be determined as the half the proportion of the inhalation time Ti that has elapsed since the previous trigger instant, or 0.5 revolutions plus half the proportion of the exhalation time Te that has elapsed since the previous cycle instant (whichever instant was more recent).

### 5.4.3.2.2 Waveform Determination

In one form of the present technology, the therapy parameter determination algorithm 4329 provides an approximately constant treatment pressure throughout a respiratory cycle of a patient.

In other forms of the present technology, the therapy control module 4330 controls the pressure generator 4140 to provide a treatment pressure Pt that varies as a function of phase Φ of a respiratory cycle of a patient according to a waveform template Π(Φ).

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template Π(Φ) is a square-wave template, having a value of 1 for values of phase up to and including 0.5 revolutions, and a value of 0 for values of phase above 0.5 revolutions. In one form, suitable for continuously-valued phase, the waveform template Π(Φ) comprises two smoothly curved portions, namely a smoothly curved (e.g. raised cosine) rise from 0 to 1 for values of phase up to 0.5 revolutions, and a smoothly curved (e.g. exponential) decay from 1 to 0 for values of phase above 0.5 revolutions. In one form, suitable for continuously-valued phase, the waveform template Π(Φ) is based on a square wave, but with a smooth rise from 0 to 1 for values of phase up to a “rise time” that is less than 0.5 revolutions, and a smooth fall from 1 to 0 for values of phase within a “fall time” after 0.5 revolutions, with a “fall time” that is less than 0.5 revolutions.

In some forms of the present technology, the waveform determination algorithm 4322 selects a waveform template Π(Φ) from a library of waveform templates, dependent on a setting of the RPT device. Each waveform template Π(Φ) in the library may be provided as a lookup table of values Π against phase values Φ. In other forms, the waveform determination algorithm 4322 computes a waveform template Π(Φ) “on the fly” using a predetermined functional form, possibly parametrised by one or more parameters (e.g. time constant of an exponentially curved portion). The parameters of the functional form may be predetermined or dependent on a current state of the patient 1000.

In some forms of the present technology, suitable for discrete bi-valued phase of either inhalation (Φ=0 revolutions) or exhalation (Φ=0.5 revolutions), the waveform determination algorithm 4322 computes a waveform template Π “on the fly” as a function of both discrete phase Φ and time t measured since the most recent trigger instant. In one such form, the waveform determination algorithm 4322 computes the waveform template Π(Φ, t) in two portions (inspiratory and expiratory) as follows:

$$Π ( Φ , t ) = \{ \begin{matrix}{Π}_{i} ( t ) , & Φ = 0 \\ {Π}_{e} ( t - {T}_{i} ) , & Φ = 0.5\end{matrix}$$

where Πᵢ(t) and Π_{c}(t) are inspiratory and expiratory portions of the waveform template Π(Φ, t). In one such form, the inspiratory portion Πᵢ(t) of the waveform template is a smooth rise from 0 to 1 parametrised by a rise time, and the expiratory portion Πₑ(t) of the waveform template is a smooth fall from 1 to 0 parametrised by a fall time.

### 5.4.3.2.3 Ventilation Determination

In one form of the present technology, a ventilation determination algorithm 4323 receives an input a respiratory flow rate Qr, and determines a measure indicative of current patient ventilation, Vent.

In some implementations, the ventilation determination algorithm 4323 determines a measure of ventilation Vent that is an estimate of actual patient ventilation. One such implementation is to take half the absolute value of respiratory flow rate, Or, optionally filtered by low-pass filter such as a second order Bessel low-pass filter with a corner frequency of 0.11 Hz.

In other implementations, the ventilation determination algorithm 4323 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate shape is constant.

### 5.4.3.2.4 Determination of Inspiratory Flow Limitation

In one form of the present technology, the central controller 4230 executes an inspiratory flow limitation determination algorithm 4324 for the determination of the extent of inspiratory flow limitation.

In one form, the inspiratory flow limitation determination algorithm 4324 receives as an input a respiratory flow rate signal Qr and provides as an output a metric of the extent to which the inspiratory portion of the breath exhibits inspiratory flow limitation.

In one form of the present technology, the inspiratory portion of each breath is identified by a zero-crossing detector. A number of evenly spaced points (for example, sixty-five), representing points in time, are interpolated by an interpolator along the inspiratory flow rate-time curve for each breath. The curve described by the points is then scaled by a scalar to have unity length (duration/period) and unity area to remove the effects of changing breathing rate and depth. The scaled breaths are then compared in a comparator with a pre-stored template representing a normal unobstructed breath, similar to the inspiratory portion of the breath shown in FIG. 6A. Breaths deviating by more than a specified threshold (typically 1 scaled unit) at any time during the inspiration from this template, such as those due to coughs, sighs, swallows and hiccups, as determined by a test element, are rejected. For non-rejected data, a moving average of the first such scaled point is calculated by the central controller 4230 for the preceding several inspiratory events. This is repeated over the same inspiratory events for the second such point, and so on. Thus, for example, sixty-five scaled data points are generated by the central controller 4230, and represent a moving average of the preceding several inspiratory events, e.g., three events. The moving average of continuously updated values of the (e.g., sixty-five) points are hereinafter called the “scaled flow rate”, designated as Qs(t). Alternatively, a single inspiratory event can be utilised rather than a moving average.

From the scaled flow rate, two shape factors relating to the determination of partial obstruction may be calculated.

Shape factor 1 is the ratio of the mean of the middle (e.g. thirty-two) scaled flow rate points to the mean overall (e.g. sixty-five) scaled flow rate points. Where this ratio is in excess of unity, the breath will be taken to be normal. Where the ratio is unity or less, the breath will be taken to be obstructed. A ratio of about 1.17 is taken as a threshold between partially obstructed and unobstructed breathing, and equates to a degree of obstruction that would permit maintenance of adequate oxygenation in a typical patient.

Shape factor 2 is calculated as the RMS deviation from unit scaled flow rate, taken over the middle (e.g. thirty-two) points. An RMS deviation of about 0.2 units is taken to be normal. An RMS deviation of zero is taken to be a totally flow-limited breath. The closer the RMS deviation to zero, the breath will be taken to be more flow limited.

Shape factors 1 and 2 may be used as alternatives, or in combination. In other forms of the present technology, the number of sampled points, breaths and middle points may differ from those described above. Furthermore, the threshold values can be other than those described.

### 5.4.3.2.5 Determination of Apneas and Hypopneas

In one form of the present technology, the central controller 4230 executes an apnea/hypopnea determination algorithm 4325 for the determination of the presence of apneas and/or hypopneas.

In one form, the apnea/hypopnea determination algorithm 4325 receives as an input a respiratory flow rate signal Qr and provides as an output a flag that indicates that an apnea or a hypopnea has been detected.

In one form, an apnea will be said to have been detected when a function of respiratory flow rate Or falls below a flow rate threshold for a predetermined period of time. The function may determine a peak flow rate, a relatively short-term mean flow rate, or a flow rate intermediate of relatively short-term mean and peak flow rate, for example an RMS flow rate. The flow rate threshold may be a relatively long-term measure of flow rate.

In one form, a hypopnea will be said to have been detected when a function of respiratory flow rate Qr falls below a second flow rate threshold for a predetermined period of time. The function may determine a peak flow, a relatively short-term mean flow rate, or a flow rate intermediate of relatively short-term mean and peak flow rate, for example an RMS flow rate. The second flow rate threshold may be a relatively long-term measure of flow rate. The second flow rate threshold is greater than the flow rate threshold used to detect apneas.

### 5.4.3.2.6 Determination of Snore

In one form of the present technology, the central controller 4230 executes one or more snore determination algorithms 4326 for the determination of the extent of snore.

In one form, the snore determination algorithm 4326 receives as an input a respiratory flow rate signal Or and provides as an output a metric of the extent to which snoring is present.

The snore determination algorithm 4326 may comprise the step of determining the intensity of the flow rate signal in the range of 30-300 Hz. Further, the snore determination algorithm 4326 may comprise a step of filtering the respiratory flow rate signal Or to reduce background noise, e.g., the sound of airflow in the system from the blower.

### 5.4.3.2.7 Determination of Airway Patency

In one form of the present technology, the central controller 4230 executes one or more airway patency determination algorithms 4327 for the determination of the extent of airway patency.

In one form, the airway patency determination algorithm 4327 receives as an input a respiratory flow rate signal Qr, and determines the power of the signal in the frequency range of about 0.75 Hz and about 3 Hz. The presence of a peak in this frequency range is taken to indicate an open airway. The absence of a peak is taken to be an indication of a closed airway.

In one form, the frequency range within which the peak is sought is the frequency of a small forced oscillation in the treatment pressure Pt. In one implementation, the forced oscillation is of frequency 2 Hz with amplitude about 1 cmH₂O.

In one form, airway patency determination algorithm 4327 receives as an input a respiratory flow rate signal Qr, and determines the presence or absence of a cardiogenic signal. The absence of a cardiogenic signal is taken to be an indication of a closed airway.

### 5.4.3.2.8 Determination of Target Ventilation

In one form of the present technology, the central controller 4230 takes as input the measure of current ventilation, Vent, and executes one or more target ventilation determination algorithms 4328 for the determination of a target value Vtgt for the measure of ventilation.

In some forms of the present technology, there is no target ventilation determination algorithm 4328, and the target value Vtgt is predetermined, for example by hard-coding during configuration of the RPT device 4000 or by manual entry through the input device 4220.

In other forms of the present technology, such as adaptive servo-ventilation (ASV), the target ventilation determination algorithm 4328 computes a target value Vtgt from a value Vtyp indicative of the typical recent ventilation of the patient.

In some forms of adaptive servo-ventilation, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4328, the recent history is of the order of several minutes, but in any case should be longer than the timescale of Cheyne-Stokes waxing and waning cycles. The target ventilation determination algorithm 4328 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 5.4.3.2.9 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt using the equation

$$P t = A Π ( Φ , t ) + {P}_{0} \qquad ( 1 )$$

where:
A is the amplitude,
Π(Φ, t) is the waveform template value (in the range 0 to 1) at the current value Φ of phase and t of time, and
P₀ is a base pressure.

If the waveform determination algorithm 4322 provides the waveform template Π(Φ, t) as a lookup table of values Π indexed by phase Φ, the therapy parameter determination algorithm 4329 applies equation (1) by locating the nearest lookup table entry to the current value Φ of phase returned by the phase determination algorithm 4321, or by interpolation between the two entries straddling the current value Φ of phase.

The values of the amplitude A and the base pressure P₀ may be set by the therapy parameter determination algorithm 4329 depending on the chosen respiratory pressure therapy mode in the manner described below.

### 5.4.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface 3000 or 3800 is equal to the treatment pressure Pt.

### 5.4.3.4 Detection of Fault Conditions

In one form of the present technology, the central controller 4230 executes one or more methods 4340 for the detection of fault conditions. The fault conditions detected by the one or more methods 4340 may include at least one of the following:
Power failure (no power, or insufficient power)
Transducer fault detection
Failure to detect the presence of a component
Operating parameters outside recommended ranges (e.g. pressure, flow rate, temperature, PaO2)
Failure of a test alarm to generate a detectable alarm signal.

Upon detection of the fault condition, the corresponding algorithm 4340 signals the presence of the fault by one or more of the following:

Initiation of an audible, visual &/or kinetic (e.g. vibrating) alarm
Sending a message to an external device
Logging of the incident

### 5.5 AIR CIRCUIT

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 5.5.1 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 5.6 HUMIDIFIER

### 5.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.6.2 Humidifier Components

### 5.6.2.1 Water Reservoir

According to one arrangement, the humidifier 5000 may comprise a water reservoir 5110 configured to hold, or retain, a volume of liquid (e.g. water) to be evaporated for humidification of the flow of air. The water reservoir 5110 may be configured to hold a predetermined maximum volume of water in order to provide adequate humidification for at least the duration of a respiratory therapy session, such as one evening of sleep. Typically, the reservoir 5110 is configured to hold several hundred millilitres of water, e.g. 300 millilitres (ml), 325 ml, 350 ml or 400 ml. In other forms, the humidifier 5000 may be configured to receive a supply of water from an external water source such as a building's water supply system.

According to one aspect, the water reservoir 5110 is configured to add humidity to a flow of air from the RPT device 4000 as the flow of air travels therethrough. In one form, the water reservoir 5110 may be configured to encourage the flow of air to travel in a tortuous path through the reservoir 5110 while in contact with the volume of water therein.

According to one form, the reservoir 5110 may be removable from the humidifier 5000, for example in a lateral direction as shown in FIG. 5A and FIG. 5B.

The reservoir 5110 may also be configured to discourage egress of liquid therefrom, such as when the reservoir 5110 is displaced and/or rotated from its normal, working orientation, such as through any apertures and/or in between its sub-components. As the flow of air to be humidified by the humidifier 5000 is typically pressurised, the reservoir 5110 may also be configured to prevent losses in pneumatic pressure through leak and/or flow impedance.

### 5.6.2.2 Conductive Portion

According to one arrangement, the reservoir 5110 comprises a conductive portion 5120 configured to allow efficient transfer of heat from the heating element 5240 to the volume of liquid in the reservoir 5110. In one form, the conductive portion 5120 may be arranged as a plate, although other shapes may also be suitable. All or a part of the conductive portion 5120 may be made of a thermally conductive material such as aluminium (e.g. approximately 2 mm thick, such as 1 mm, 1.5 mm, 2.5 mm or 3 mm), another heat conducting metal or some plastics. In some cases, suitable heat conductivity may be achieved with less conductive materials of suitable geometry.

### 5.6.2.3 Humidifier Reservoir Dock

In one form, the humidifier 5000 may comprise a humidifier reservoir dock 5130 (as shown in FIG. 5B) configured to receive the humidifier reservoir 5110. In some arrangements, the humidifier reservoir dock 5130 may comprise a locking feature such as a locking lever 5135 configured to retain the reservoir 5110 in the humidifier reservoir dock 5130.

### 5.6.2.4 Water Level Indicator

The humidifier reservoir 5110 may comprise a water level indicator 5150 as shown in FIG. 5A-5B. In some forms, the water level indicator 5150 may provide one or more indications to a user such as the patient 1000 or a care giver regarding a quantity of the volume of water in the humidifier reservoir 5110. The one or more indications provided by the water level indicator 5150 may include an indication of a maximum, predetermined volume of water, any portions thereof, such as 25%, 50% or 75% or volumes such as 200 ml, 300 ml or 400 ml.

### 5.6.2.5 Humidifier Transducer(s)

The humidifier 5000 may comprise one or more humidifier transducers (sensors) 5210 instead of, or in addition to, transducers 4270 described above. Humidifier transducers 5210 may include one or more of an air pressure sensor 5212, an air flow rate transducer 5214, a temperature sensor 5216, or a humidity sensor 5218 as shown in FIG. 5C. A humidifier transducer 5210 may produce one or more output signals which may be communicated to a controller such as the central controller 4230 and/or the humidifier controller 5250. In some forms, a humidifier transducer may be located externally to the humidifier 5000 (such as in the air circuit 4170) while communicating the output signal to the controller.

### 5.6.2.5.1 Pressure Transducer

One or more pressure transducers 5212 may be provided to the humidifier 5000 in addition to, or instead of, a pressure sensor 4272 provided in the RPT device 4000.

### 5.6.2.5.2 Flow Rate Transducer

One or more flow rate transducers 5214 may be provided to the humidifier 5000 in addition to, or instead of, a flow rate sensor 4274 provided in the RPT device 4000.

5.6.2.5.3 Temperature transducer

The humidifier 5000 may comprise one or more temperature transducers 5216. The one or more temperature transducers 5216 may be configured to measure one or more temperatures such as of the heating element 5240 and/or of the flow of air downstream of the humidifier outlet 5004. In some forms, the humidifier 5000 may further comprise a temperature sensor 5216 to detect the temperature of the ambient air.

### 5.6.2.5.4 Humidity Transducer

In one form, the humidifier 5000 may comprise one or more humidity sensors 5218 to detect a humidity of a gas, such as the ambient air. The humidity sensor 5218 may be placed towards the humidifier outlet 5004 in some forms to measure a humidity of the gas delivered from the humidifier 5000. The humidity sensor may be an absolute humidity sensor or a relative humidity sensor.

### 5.6.2.6 Heating Element

A heating element 5240 may be provided to the humidifier 5000 in some cases to provide a heat input to one or more of the volume of water in the humidifier reservoir 5110 and/or to the flow of air. The heating element 5240 may comprise a heat generating component such as an electrically resistive heating track. One suitable example of a heating element 5240 is a layered heating element such as one described in the PCT Patent Application Publication No. WO 2012/171072, which is incorporated herewith by reference in its entirety.

In some forms, the heating element 5240 may be provided in the humidifier base 5006 where heat may be provided to the humidifier reservoir 5110 primarily by conduction as shown in FIG. 5B.

### 5.6.2.7 Humidifier Controller

According to one arrangement of the present technology, a humidifier 5000 may comprise a humidifier controller 5250 as shown in FIG. 5C. In one form, the humidifier controller 5250 may be a part of the central controller 4230. In another form, the humidifier controller 5250 may be a separate controller, which may be in communication with the central controller 4230.

In one form, the humidifier controller 5250 may receive as inputs measures of properties (such as temperature, humidity, pressure and/or flow rate), for example of the flow of air, the water in the reservoir 5110 and/or the humidifier 5000. The humidifier controller 5250 may also be configured to execute or implement humidifier algorithms and/or deliver one or more output signals.

As shown in FIG. 5C, the humidifier controller 5250 may comprise one or more controllers, such as a central humidifier controller 5251, a heated air circuit controller 5254 configured to control the temperature of a heated air circuit 4171 and/or a heating element controller 5252 configured to control the temperature of a heating element 5240.

### 5.7 BREATHING WAVEFORMS

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

FIG. 6B shows selected polysomnography channels (pulse oximetry, flow rate, thoracic movement, and abdominal movement) of a patient during non-REM sleep breathing normally over a period of about ninety seconds, with about 34 breaths, being treated with automatic PAP therapy, and the interface pressure being about 11 cmH2O. The top channel shows pulse oximetry (oxygen saturation or SpO2), the scale having a range of saturation from 90 to 99% in the vertical direction. The patient maintained a saturation of about 95% throughout the period shown. The second channel shows quantitative respiratory flow rate, and the scale ranges from −1 to +1 LPS in a vertical direction, and with inspiration positive. Thoracic and abdominal movement are shown in the third and fourth channels.

FIG. 6C shows polysomnography of a patient before treatment. There are eleven signal channels from top to bottom with a 6-minute horizontal span. The top two channels are both EEG (electroencephalogram) from different scalp locations. Periodic spikes in the second EEG represent cortical arousal and related activity. The third channel down is submental EMG (electromyogram). Increasing activity around the time of arousals represents genioglossus recruitment. The fourth & fifth channels are EOG (electro-oculogram). The sixth channel is an electrocardiogram. The seventh channel shows pulse oximetry (SpO2) with repetitive desaturations to below 70% from about 90%. The eighth channel is respiratory flow rate using a nasal cannula connected to a differential pressure transducer. Repetitive apneas of 25 to 35 seconds alternate with 10 to 15 second bursts of recovery breathing coinciding with EEG arousal and increased EMG activity. The ninth channel shows movement of chest and the tenth shows movement of abdomen. The abdomen shows a crescendo of movement over the length of the apnea leading to the arousal. Both become untidy during the arousal due to gross body movement during recovery hyperpnea. The apneas are therefore obstructive, and the condition is severe. The lowest channel is posture, and in this example it does not show change.

FIG. 6D shows patient flow rate data where the patient is experiencing a series of total obstructive apneas. The duration of the recording is approximately 160 seconds. Flow rates range from about +1 L/s to about −1.5 L/s. Each apnea lasts approximately 10-15 s.

FIG. 6E shows a scaled inspiratory portion of a breath where the patient is experiencing low frequency inspiratory snore.

FIG. 6F shows a scaled inspiratory portion of a breath where the patient is experiencing an example of flattened inspiratory flow limitation.

FIG. 6G shows a scaled inspiratory portion of a breath where the patient is experiencing an example of “mesa” flattened inspiratory flow limitation.

FIG. 6H shows a scaled inspiratory portion of a breath where the patient is experiencing an example of “panda ears” inspiratory flow limitation.

FIG. 6I shows a scaled inspiratory portion of a breath where the patient is experiencing an example of “chair” inspiratory flow limitation.

FIG. 6J shows a scaled inspiratory portion of a breath where the patient is experiencing an example of “reverse chair” inspiratory flow limitation.

FIG. 6K shows a scaled inspiratory portion of a breath where the patient is experiencing an example of “M-shaped” inspiratory flow limitation.

FIG. 6L shows a scaled inspiratory portion of a breath where the patient is experiencing an example of severely “M-shaped” inspiratory flow limitation.

FIG. 6M shows patient data from a patient with Cheyne-Stokes respiration. There are three channels: pulse oximetry (SpO2); a signal indicative of flow rate; and thoracic movement. The data span six minutes. The signal representative of flow rate was measured using a pressure sensor connected to a nasal cannula. The patient exhibits apneas of about 22 seconds and hyperpneas of about 38 seconds. The higher frequency low amplitude oscillation during apnea is cardiogenic.

FIG. 6N shows patient data from a patient with another example of Cheyne-Stokes respiration, using the same three channels as in FIG. 6M. The data span ten minutes. The patient exhibits hyperpneas of about 30 seconds and hypopneas of about 30 seconds.

### 5.8 SCREENING, DIAGNOSIS, MONITORING SYSTEMS

### 5.8.1 Polysomnography

FIG. 7A shows a patient 1000 undergoing polysomnography (PSG). A PSG system comprises a headbox 2000 which receives and records signals from the following sensors: an EOG electrode 2015; an EEG electrode 2020; an ECG electrode 2025; a submental EMG electrode 2030; a snore sensor 2035; a respiratory inductance plethysmogram (respiratory effort sensor) 2040 on a chest band; a respiratory inductance plethysmogram (respiratory effort sensor) 2045 on an abdominal band; an oro-nasal cannula 2050 with oral thermistor; a photoplethysmograph (pulse oximeter) 2055; and a body position sensor 2060. The electrical signals are referred to a ground electrode (ISOG) 2010 positioned in the centre of the forehead.

### 5.8.2 Non-Obtrusive Monitoring System

One example of a monitoring apparatus 7100 for monitoring the respiration of a sleeping patient 1000 is illustrated in FIG. 7B. The monitoring apparatus 7100 contains a contactless motion sensor generally directed toward the patient 1000. The motion sensor is configured to generate one or more signals representing bodily movement of the patient 1000, from which may be obtained a signal representing respiratory movement of the patient.

### 5.8.3 Respiratory Polygraphy

Respiratory polygraphy (RPG) is a term for a simplified form of PSG without the electrical signals (EOG, EEG, EMG), snore, or body position sensors. RPG comprises at least a thoracic movement signal from a respiratory inductance plethysmogram (movement sensor) on a chest band, e.g. the movement sensor 2040, a nasal pressure signal sensed via a nasal cannula, and an oxygen saturation signal from a pulse oximeter, e.g. the pulse oximeter 2055. The three RPG signals, or channels, are received by an RPG headbox, similar to the PSG headbox 2000.

In certain configurations, a nasal pressure signal is a satisfactory proxy for a nasal flow rate signal generated by a flow rate transducer in-line with a sealed nasal mask, in that the nasal pressure signal is comparable in shape to the nasal flow rate signal. The nasal flow rate in turn is equal to the respiratory flow rate if the patient's mouth is kept closed, i.e. in the absence of mouth leaks.

FIG. 7C is a block diagram illustrating a screening/diagnosis/monitoring device 7200 that may be used to implement an RPG headbox in an RPG screening/diagnosis/monitoring system. The screening/diagnosis/monitoring device 7200 receives the three RPG channels mentioned above (a signal indicative of thoracic movement, a signal indicative of nasal flow rate, and a signal indicative of oxygen saturation) at a data input interface 7260. The screening/diagnosis/monitoring device 7200 also contains a processor 7210 configured to carry out encoded instructions. The screening/diagnosis/monitoring device 7200 also contains a non-transitory computer readable memory/storage medium 7230.

Memory 7230 may be the screening/diagnosis/monitoring device 7200's internal memory, such as RAM, flash memory or ROM. In some implementations, memory 7230 may also be a removable or external memory linked to screening/diagnosis/monitoring device 7200, such as an SD card, server, USB flash drive or optical disc, for example. In other implementations, memory 7230 can be a combination of external and internal memory. Memory 7230 includes stored data 7240 and processor control instructions (code) 7250 adapted to configure the processor 7210 to perform certain tasks. Stored data 7240 can include RPG channel data received by data input interface 7260, and other data that is provided as a component part of an application. Processor control instructions 7250 can also be provided as a component part of an application program. The processor 7210 is configured to read the code 7250 from the memory 7230 and execute the encoded instructions. In particular, the code 7250 may contain instructions adapted to configure the processor 7210 to carry out methods of processing the RPG channel data provided by the interface 7260. One such method may be to store the RPG channel data as data 7240 in the memory 7230. Another such method may be to analyse the stored RPG data to extract features. The processor 7210 may store the results of such analysis as data 7240 in the memory 7230.

The screening/diagnosis/monitoring device 7200 may also contain a communication interface 7220. The code 7250 may contain instructions configured to allow the processor 7210 to communicate with an external computing device (not shown) via the communication interface 7220. The mode of communication may be wired or wireless. In one such implementation, the processor 7210 may transmit the stored RPG channel data from the data 7240 to the remote computing device. In such an implementation, the remote computing device may be configured to analyse the received RPG data to extract features. In another such implementation, the processor 7210 may transmit the analysis results from the data 7240 to the remote computing device.

Alternatively, if the memory 7230 is removable from the screening/diagnosis/monitoring device 7200, the remote computing device may be configured to be connected to the removable memory 7230. In such an implementation, the remote computing device may be configured to analyse the RPG data retrieved from the removable memory 7230 to extract the features.

### 5.8.4 Systems for Analysing Sensing Kit Data

The first circuit board assembly 8030 or the second circuit board assembly 9000, 10020, 11020 may include a communication module, such as a Bluetooth or other type of wired or wireless module, for communicating with an external device, such as a mobile device (e.g., phone or tablet) or a computer (e.g., a laptop). Although the below discussion references the sensing kit 8000, the first circuit board assembly 8030, and the second circuit board assembly 9000, it will be appreciated that the below discussion applies equally to the second circuit board assemblies 10020, 11020.

As shown in FIG. 12A, the sensing kit 8000 may be in wireless communication with an external device 12002 in a reporting system 12000. Although the sensing kit 8000 is shown in Bluetooth communication with the external device 12002, it will be appreciated that sensing kit 8000 may communicate with the external device 12002 by other mechanisms, including, for example, wired or wireless network connections. Although the external device 12000 is depicted as a laptop computer, it will be appreciated that any type of external device may be utilized.

The external device 12002 may have software installed thereon that is configured to interact with the sensing kit 8000. For example, the external device 12002 may include a memory having instructions stored thereon for execution by a processor. Additionally or alternatively, the external device 12002 may be configured to access remotely-stored software (e.g., via a network). The software may provide a user interface 12010 to allow a user to interact with the sensing kit 8000. Using the user interface 12010, the user may, for example, turn elements of the sensing kit 8000 on/off, check a battery life of the sensing kit 8000, confirm whether the components of the sensing kit 8000 are connected, visualize data from the sensing kit 8000 (e.g., live data), and manage files (e.g., files to be stored on external device 12002 of data from the sensing data 8000).

The external device 12002 also may be configured to interface with other types of devices, such as, for example, the RPT device 4000, the humidifier 5000, or other types of sensors (e.g., sensors of the patient interface 3000, 8010, 10000, 11000) or sensors in patches applied to the patient. Examples of such external sensors may be found in, for example, PCT Application No. PCT/IB2021/052557, filed Mar. 27, 2021, Australian Provisional Application No. 2022900799, filed Mar. 30, 2022, and PCT Application No. PCT/US2020/044632, filed on Jul. 31, 2022, each of which is incorporated herein in its entirety. The user interface 12010 may be configured to host such other devices, and may synchronize data from such devices with data from sensing kit 8000. Thus, a user may visualize and understand various aspects of a patient's breathing experience while using the RPT device 4000 and the patient interface 3000, 8010, 10000, 11000. In aspects, the software of the external device 12002 may align time-stamped data from the RPT device 4000 (e.g., flow, set pressure, and/or device settings), device from other external sensors (e.g., data reflecting patient breathing effort), and in-mask conditions or other parameters measured by the sensing kit 8000. Data received from sensing kit 8000, the RPT device 4000, other types of sensors, or any other source may be stored locally on a particular external device 12002 or may be stored, for example, in a cloud-based system (e.g., on a server), so that multiple external devices 12002 can access the stored data.

A first screen 12020 of the user interface (FIG. 12B) may include a pane 12022 for indicating connection statuses of various devices. Although the pane 12022 is shown on the left-hand side of the first screen 12020, it will be appreciated that the pane 12022 may be positioned anywhere on the user interface 12010. The pane 12022 may identify devices (e.g., devices that have been selected by the user, devices that are available for connection, or a set of devices otherwise generated). For example, as shown in FIG. 12B, devices may include a flow generator such as the RPT device 4000, a patch sensor, and sensing kit 8000. The pane 12022 may identify a connection status of a device (e.g., connected or disconnected), a serial number of the device, a battery status of the device, a status (e.g., a memory status) of the device, and/or a memory usage/data usage of the device. The user pane 12022 may include various buttons for interacting with the user interface 12010 (e.g., a “connect” button for connecting to a disconnected device, a “live view” button for viewing a live data stream from a device, a “file manager” button for managing data files, a “reset” button for resetting one or more parameters of a device, and a “disconnect” button for disconnecting from a connected device. The buttons described above are merely exemplary, and any type of button may be utilized. Data of the pane 12022 may be obtained automatically by the user interface 12010 upon connection/syncing with a device. The sensors (e.g., patch sensors or sensing kit 8000) may be configured to automatically turn on when connected to the external device 12002 and to turn off when disconnected from the external device 12002.

The first screen 12020 of the user interface 12010 may include a setup test pane 12024. The setup test pane 12024 may include fields for entering a test name, a participant ID, another ID, and selections for types of user interfaces, tubing, ramp settings for the RPT device 4000, an expiratory pressure relief (“EPR”) setting of the RPT device 4000, or any other settings of the RPT device 4000 (e.g., pressure). Such settings of the RPT device 4000 may be input directly to the RPT device 4000 and entered into or detected by the user interface 12010. Alternatively or additionally, the user interface 12010 may be used to control parameters of the RPT device 4000. For example, a setting may be entered into the user interface 12010, and the external device 12002 may control the RPT device 4000. The user interface 12010 also may allow saving of “favorite” configurations of the RPT device 4000 for easy switching between various settings.

The setup test pane 12024 or another portion of the user interface 12010 also may include a button to start recording of the connected sensors (e.g., sensors of sensing kit 8000) or to manage files relating to the test. Data may begin to be collected when a first button (e.g., a “start test” or “start recording”) is activated and may be halted when a second button (e.g., a “stop test” or “stop recording”) button is activated or when the first button is deactivated. As discussed above, data may be live streamed as it is recorded. A time limit may apply to how long the live stream may be viewed (e.g., for up to five minutes). Data obtained from the sensing kit 8000 may be stored on, for example, first circuit board 8032. Data from the RPT device 4000 may be stored on the RPT device 4000 or on the external device 12002. Data from other types of external sensors (e.g., a patch sensor) may be stored on the external sensor. Additionally or alternatively, data may from sensing kit 8000 or another sensor may be stored on, for example, the external device 12002 or remotely (e.g., in a cloud computing environment) if the sensing kit 8000 or another sensor is connected (e.g., via a wireless or wired connection) to the external device 12002 in such a way that such data may be transferred.

In some aspects, after the data is collected, the sensing kit 8000 may be connected to the docking station 8070. The docking station 8070 may be connected to the external device 12002 to transfer the data to the external device 12002. Additionally or alternatively, the sensing kit 8000 may be connected to the external device 12002, via, for example a cable, such as a USB cable in order to transfer data from the sensing kit 8000 to the external device 12002. Other sensors (e.g., patch sensors) may be connected to the docking station 8070 or to another docking station, or may otherwise transfer data stored thereon. When the data is uploaded from the sensing kit 8000 and/or other sensors, the data may be named and timestamped to permit coordination of data from multiple sources (e.g., from the RPT device 4000, the sensing kit 8000, and other external sensors). For example, files from the different devices may be grouped together in the user interface 12010.

The user interface 12010 may provide a dashboard for viewing and analysing data from the various sources. For example, high-resolution data captured can be rendered in a range of customisable graphs, charts and plots, depending on the data to be analyzed. The user interface 12010 may provide for fields where subjective opinion can be manually added. For instance, pressure within the plenum chamber 8012 (or other values measured by sensors of sensing kit 8000) may be compared to data from the RPT device 4000 (e.g., flow generation data) and a measure of respiratory effort obtained from a sensor (e.g., on a patch) on the patient, over time, within a single graph. Factors such as scale, time period, labels, colors, etc. may be customisable, allowing for unique analysis of each dataset. Any type of plot, table, or graph may be rendered, based on a type and amount of data collected.

A screen 12030 (FIG. 12C) of user interface 12010 may provide live data or an overview of stored data from sensing kit 8000. For example, a plot 12032 may show information about measurements of CO₂ obtained from a CO₂ sensor of sensing kit 8000. A plot 12034 may show information about temperature measurements obtained from a temperature sensor of sensing kit 8000. A plot 12036 may display pressure measurements obtained from a pressure sensor of sensing kit 8000. A plot 12038 may present relative humidity measurements obtained from a humidity sensor of sensing kit 8000.

Other screens 12040 (FIG. 12D), 12050 (FIG. 12E), 12060 (FIG. 12F), 12070 (FIG. 12G), 12080 (FIG. 12H), and 12090 (FIG. 12I) of the user interface 12010 may depict other representations of data obtained from sensing kit 8000, the RPT device 4000, or other sensors (e.g., sensors from a patch applied to a patient). The graphs, plots, or other data representations may depict data from one or more sources, which may be overlaid or otherwise presented to synthesize the sources of data. The data representations described below are merely exemplary, and reporting system 12000 may generate any suitable type of data representation, report, or analysis.

The screen 12040, depicted in FIG. 12D, may plot pressure versus time on a graph 12048. For example, the pressure sensor 9012 of the second circuit board assembly 9000 may measure the pressure plotted on the graph 12048. The screen 12040 may include a data selection pane 12042, with which a user may select, for example, a dataset to analyze, a type of mask, and a time range for which to display data. The screen 12040 may include a flow selection interface 12044, with which a user may select a total flow, a patient flow, or a vent flow. The flows may be measured by the RPT device 4000 in some examples. A total flow may include the flow measured by RPT device 4000, which may include a patient flow and a vent flow. Patient flow may be obtained from the output of the RPT device 4000 or via high-pass filtering of the total flow using, for example, a cut-off frequency of 0.5 Hz.

As shown in FIG. 12D, total flow is selected. The screen 12040 also may include a details pane 12046, on which statistics such as maximum pressure, minimum pressure, and pressure swing (a difference between the maximum pressure and minimum pressure) may be displayed. The details pane 12046 also may include a key indicating certain data points of the graph 12048, such as a first dot of the graph 12048 indicating the maximum pressure, a second dot of the graph 12048 indicating the minimum pressure, and a dotted line of the graph 12048 depicting the extent of the pressure swing.

The screen 12050 (FIG. 12E) may depict various graphs 12052, 12054, 12056 showing patient flow. In some examples, the screen 12040 and the screen 12050 may be portions of the same screen, with the screen 12040 depicting a top portion of the screen and the screen 12050 depicting a bottom portion of the screen, after scrolling down from the screen 12040. The data selections described with respect to screen 12040 also may apply to the screen 12050. The graph 12052 may depict patient flow versus time. As shown in a key 12053, different colored dots of the graph 12052 may indicate inspiration start and expiration start. The graph 12052 may be used to determine minute respiratory rate, tidal volume (integral of flow with respect to time), and/or minute ventilation. The graph 12052 and the values derived therefrom may reflect how steadily a patient is breathing which may be an indicator of, for example, stress, insomnia, or sleep onset. The graph 12052 also may be analyzed to find if any other respiratory conditions are present. The graphs 12054 and 12056 may depict patient flow breath by breath versus time. The graph 12054 may overlay breaths of the graph 12052 (e.g., periods between inspiration starts) upon one another. For example, the graph 12054 may depict 14 breaths, depicted in different colors, as indicated by a key 12055. The graph 12054 may be analyzed to compare breaths for consistency and to visualize metrics such as minute respiratory rate, tidal volume, minute ventilation, etc. The graph 12056 may depict respiration rate (breaths per minute), minute ventilation (breaths*liters/minute), and total volume (liters), as shown in a key 12057. The graph 12056 may facilitate comparison of the mean, standard deviation, and normal distribution of some or all of the parameters shown (e.g., respiration rate, tidal volume, minute ventilation, and pressure swings) across multiple patients or tests for a particular window of time. The graph 12056 may assist with understanding the influence of each of these factors on one another and identifying the key contributing factors to breathing comfort. The graph 12056 also may help to identify which independent variables (e.g., factors relating to flow, such as respiration rate, tidal volume, minute ventilation, or others) have the largest impacts on the dependent variables (e.g., pressure swings).

The screen 12060 (FIG. 12F) may depict graphs 12062, 12064 showing statistics of interest. The screen 12060 may include a data selection pane 12066, with which a user may select, for example, a dataset to analyze, a type of mask, and a time range for which to display data. As shown on screen 12060, two datasets are selected. These datasets may be the test data that is analyzed and shown in the user interface 12010. The data may be for a single test (1 entry) or across multiple entries/patient test data. The graph 12062 may depict a distribution plot of respiratory rate. As shown in key 12063, a first of the selected datasets may be analyzed in a first color, and a second of the selected datasets may be analyzed in a second color. A bar graph may be displayed for each of the datasets of graph 12062, along with a fit curve. A plot 12066 may include a hash for each measured respiratory rate (with the respiratory rates on the X-axis). The data of the plot 12066 may underlie the distribution plot of the graph 12062. The graph 12064 may depict a distribution plot of tidal volume. As shown in a key 12065, a first of the selected datasets may be analyzed in a first color, and a second of the selected datasets may be analyzed in a second color. A bar graph may be displayed for each of the datasets of the graph 12064, along with a fit curve. A plot 12068 may include a hash for each measured tidal volume (with the tidal volume on the X-axis).

The screen 12070 (FIG. 12G) may depict a graph 12072 of pressure vs flow, with exhale flow being on the negative x-axis, and inhale flow being on the positive x-axis. In some examples, the screen 12060 and the screen 12070 may be portions of the same screen, with the screen 12060 depicting a top portion of the screen and the screen 12070 depicting a bottom portion of the screen, after scrolling down from the screen 12060. The data selections described with respect to the screen 12060 also may apply to screen 12070. As shown in key 12074, one color may depict an inspiration and another color may depict a smoothed inspiration. The key may also indicate an inspiration time and a smoothed inspiration time. The shaded area of the graph 12072 may represent the work of breathing or the power of breathing (measured in Joules/seconds) and may provide a manner with which to visualise the breathing effort of a patient on pressure.

The screen 12080 (FIG. 12H) may include a graph 12082 that depicts pressure, smoothed pressure, patient flow, and smoothed patient flow at two times, as shown in key 12083. The patient flow may be obtained from the RPT device 4000 and pressure may be measured by the pressure sensor 9012 of the second circuit board assembly 9000. The smoothed pressure may obtained using a Savitzky-Golay filter on the mask pressure data from the pressure sensor 9012 and may be used to reduce some of the noise on the pressure signal to improve visualisation of, for example, the graph 12072. The times may be selected using a selection pane 12084. A summary pane 12086 may provide a summary of information depicted in graph 12082 for each of the two selected times. For example, the summary pane may provide information regarding pressure swing, flow swing, vent flow, respiration rate, total volume, and minute ventilation.

The screens discussed above are merely exemplary and other screens having other data representations also may be generated. For example, the user interface 12010 may include representations such as the following. Measurements of CO₂ levels from, for example, the CO₂ sensor 9014 of second circuit board assembly 9000 may be overlaid graphs of flows vs. time. Such a representation may enable measurement of end-tidal CO₂ values per breath and enable recording changes over time in a statistics tab. Additionally or alternatively, a waveform of CO₂ values (e.g., from the CO₂ sensor 9014) may be displayed and may facilitate determining signs of other potential respiratory illnesses. For example, the CO₂ sensor 9014 may provide breath-by-breath data on which to perform breath-by-breath analysis. For example, relationships may exist between values measured by the CO₂ sensor 9014 and the values of CO₂ in the patient airway.

In other examples, temperature and/or humidity (e.g., measured by the temperature and humidity sensor 9016) may be plotted and observed over time. Exhaled breath temperature may provide information useful to detecting and monitoring pathological process within the respiratory system. Relative humidity within the mask (e.g., measured by the temperature and humidity sensor 9016) may be indicative of breathing comfort because, for example, humidified air prevents the airways from drying out. The relationship between flow and relative humidity may be analysed to provide an indicator of how well the particular patient interface 8010, 10000, 11000 is retaining exhaled or device delivered humidified air.

In further examples, data from an accelerometer (e.g., an accelerometer of the first circuit board assembly 8030) may be utilized. For example, a gravity unit vector may be obtained from normalised accelerometer data in order to determine a patient's sleeping position at any given time. Such information may allow analysis of whether positional sleep apnea is occurring and analysis of whether any changes in CO₂ (e.g., measured by the CO₂ sensor 9014), patient flow, and/or pressure metrics inside of the plenum chamber 8012, 10012, 110012 (e.g., pressure accuracy and swings measured by the pressure sensor 9012) result from sleeping position.

For example, the screen 12090 (FIG. 12I) includes a graph 12091 of pressure (e.g., measured by the pressure sensor 9012) versus time, a graph 12092 of relative humidity (e.g., measured by the temperature and humidity sensor 9016) versus time, a graph 12093 of temperature (e.g., measured by the temperature and humidity sensor 9016) versus time, and a graph 12094 of CO₂ levels (percentages) (e.g., measured by the CO₂ sensor 9014) versus time. One or more graphs 12095 may depict various outputs (e.g., acceleration data in X, Y, and Z directions, and gyroscopic data in X, Y, and Z directions) of an accelerometer (e.g., an accelerometer of the first circuit board assembly 8030). A graph 12096 may depict a transformed gravity vector obtained from, for example, the accelerometer.

As discussed above, data from the sensing kit 8000 may be utilized in order to analyze, optimize, and/or adjust parameters of the RPT device 4000 and/or the patient interfaces 8010, 10000, 11000. Data from other data sources, such as the RPT device 4000 or other sensors (e.g., a sensor patch) also may be used to analyze, optimize, and/or adjust parameters of the RPT device 4000 and/or the patient interfaces 8010, 10000, 11000. Data from different sources (e.g., the data sensing kit 8000, the RPT device 4000, or other sensors, may be combined in any suitable fashion for such analysis, optimization, and/or adjustments. For example, data or analyses of the screens 12040 (FIG. 12D), 12050 (FIG. 12E), 12060 (FIG. 12F), 12070 (FIG. 12G), and 12080 (FIG. 12H) of user interface 12010 may be utilized.

In an exemplary method, a patient may put on the patient interface 8010, 10000, 11000 and utilize the RPT device 4000. Data may be collected by the sensing kit 8000, by the RPT device 4000, and/or by other sensors. The data may be transmitted to the external device 12002, as described above. In aspects, the external device 12002 also may receive from the patient inputs relating to breathing comfort or other parameters, as discussed above. In some examples, the patient may utilize the external device 12002 in order to input information. In other examples, the patient may utilize another device, which may transmit the information to the external device 12002.

Based on the data received from the data sensing kit 8000, the RPT device 4000, other sensors, and/or patient inputs, analysis, optimization, and/or adjustment steps may be taken. In some examples, an operator of a system such as the reporting system 12000 may analyze the data (e.g., using data representations of the screens described above) in order to determine measurements that are associated with patient reports of breathing comfort or discomfort. The analysis may be utilized to develop new devices or to develop settings for existing devices which produce improved breathing comfort.

Additionally or alternatively, the received data may be used in order to automatically or manually adjust the patient interface 8010, 10000, 11000 and/or the RPT device 4000. The reporting system 12000 may analyze the received data in order to determine appropriate changes to settings or devices. In some examples, an adjustment may be automatically made in real time. For example, a flow rate, pressure, temperature, humidity, or other parameter may be adjusted in order to increase breathing comfort of the patient. In other examples, information may be provided to the patient regarding settings to input in order to increase breathing comfort. In still other examples, recommendations may be made to the patient as to alternative types of the patient interfaces 8010, 10000, 11000 or the RPT devices 4000.

FIG. 13 depicts an example system 13000 that may execute techniques presented herein. FIG. 13 is a simplified functional block diagram of a computer that may be configured to execute techniques described herein, according to exemplary forms of the present disclosure. Specifically, the computer (or “platform” as it may not be a single physical computer infrastructure) may include a data communication interface 13060 for packet data communication. The platform may also include a central processing unit (“CPU”) 13020, in the form of one or more processors, for executing program instructions. The platform may include an internal communication bus 13010, and the platform may also include a program storage and/or a data storage for various data files to be processed and/or communicated by the platform such as a ROM 13030 and a RAM 13040, although the system 13000 may receive programming and data via network communications. The system 13000 also may include input and output ports 13050 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one form, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

### 5.9 PORTABLE OXYGEN CONCENTRATOR (POC)

Portable oxygen concentrators may take advantage of pressure swing adsorption (PSA). Pressure swing adsorption may involve using one or more compressors to increase gas pressure inside a canister that contains particles of a gas separation adsorbent arranged in a “sieve bed”. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The gas separation adsorbent may be regenerated by reducing the pressure, which reverses the adsorption of molecules from the adsorbent. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Ambient air usually includes approximately 78% nitrogen and 21% oxygen with the balance comprised of argon, carbon dioxide, water vapor and other trace gases. If a gas mixture such as air, for example, is passed under pressure through a canister containing a gas separation adsorbent bed that attracts nitrogen more strongly than it does oxygen, part or all of the nitrogen will stay in the bed, and the gas coming out of the canister will be enriched in oxygen. When the bed reaches the end of its capacity to adsorb nitrogen, it can be regenerated by reducing the pressure, thereby releasing the adsorbed nitrogen. It is then ready for another cycle of producing oxygen enriched air. By alternating canisters in a two-canister system, one canister can be separating oxygen while the other canister is being purged (resulting in a continuous separation of the oxygen from the nitrogen). In this manner, oxygen enriched air can be accumulated, such as in a storage container or other pressurizable vessel or conduit coupled to the canisters, for a variety of uses including providing supplemental oxygen to patients.

### 5.10 RESPIRATORY THERAPY MODES

Various respiratory therapy modes may be implemented by the disclosed respiratory therapy system.

In some implementations of respiratory pressure therapy, the central controller 4230 sets the treatment pressure Pt according to the treatment pressure equation (1) as part of the therapy parameter determination algorithm 4329. In one such implementation, the amplitude A is identically zero, so the treatment pressure Pt (which represents a target value to be achieved by the interface pressure Pm at the current instant of time) is identically equal to the base pressure P₀ throughout the respiratory cycle. Such implementations are generally grouped under the heading of CPAP therapy. In such implementations, there is no need for the therapy engine module 4320 to determine phase Φ or the waveform template Π(Φ).

In CPAP therapy, the base pressure P₀ may be a constant value that is hard-coded or manually entered to the RPT device 4000. Alternatively, the central controller 4230 may repeatedly compute the base pressure P₀ as a function of indices or measures of sleep disordered breathing returned by the respective algorithms in the therapy engine module 4320, such as one or more of flow limitation, apnea, hypopnea, patency, and snore. This alternative is sometimes referred to as APAP therapy

FIG. 4E is a flow chart illustrating a method 4500 carried out by the central controller 4230 to continuously compute the base pressure P₀ as part of an APAP therapy implementation of the therapy parameter determination algorithm 4329, when the pressure support A is identically zero.

The method 4500 starts at step 4520, at which the central controller 4230 compares the measure of the presence of apnea/hypopnea with a first threshold, and determines whether the measure of the presence of apnea/hypopnea has exceeded the first threshold for a predetermined period of time, indicating an apnea/hypopnea is occurring. If so, the method 4500 proceeds to step 4540; otherwise, the method 4500 proceeds to step 4530. At step 4540, the central controller 4230 compares the measure of airway patency with a second threshold. If the measure of airway patency exceeds the second threshold, indicating the airway is patent, the detected apnea/hypopnea is deemed central, and the method 4500 proceeds to step 4560; otherwise, the apnea/hypopnea is deemed obstructive, and the method 4500 proceeds to step 4550.

At step 4530, the central controller 4230 compares the measure of flow limitation with a third threshold. If the measure of flow limitation exceeds the third threshold, indicating inspiratory flow is limited, the method 4500 proceeds to step 4550; otherwise, the method 4500 proceeds to step 4560.

At step 4550, the central controller 4230 increases the base pressure P₀ by a predetermined pressure increment ΔP, provided the resulting treatment pressure Pt would not exceed a maximum treatment pressure Pmax. In one implementation, the predetermined pressure increment ΔP and maximum treatment pressure Pmax are 1 cmH2O and 25 cmH2O respectively. In other implementations, the pressure increment ΔP can be as low as 0.1 cmH2O and as high as 3 cmH2O, or as low as 0.5 cmH2O and as high as 2 cmH2O. In other implementations, the maximum treatment pressure Pmax can be as low as 15 cmH2O and as high as 35 cmH2O, or as low as 20 cmH2O and as high as 30 cmH2O. The method 4500 then returns to step 4520.

At step 4560, the central controller 4230 decreases the base pressure P₀ by a decrement, provided the decreased base pressure P₀ would not fall below a minimum treatment pressure Pmin. The method 4500 then returns to step 4520. In one implementation, the decrement is proportional to the value of P₀−Pmin, so that the decrease in P₀ to the minimum treatment pressure Pmin in the absence of any detected events is exponential. In one implementation, the constant of proportionality is set such that the time constant τ of the exponential decrease of P₀ is 60 minutes, and the minimum treatment pressure Pmin is 4 cmH2O. In other implementations, the time constant r could be as low as 1 minute and as high as 300 minutes, or as low as 5 minutes and as high as 180 minutes. In other implementations, the minimum treatment pressure Pmin can be as low as 0 cmH2O and as high as 8 cmH2O, or as low as 2 cmH2O and as high as 6 cmH2O. Alternatively, the decrement in P₀ could be predetermined, so the decrease in P₀ to the minimum treatment pressure Pmin in the absence of any detected events is linear.

### 5.10.1 Bi-Level Therapy

In other implementations of this form of the present technology, the value of amplitude A in equation (1) may be positive. Such implementations are known as bi-level therapy, because in determining the treatment pressure Pt using equation (1) with positive amplitude A, the therapy parameter determination algorithm 4329 oscillates the treatment pressure Pt between two values or levels in synchrony with the spontaneous respiratory effort of the patient 1000. That is, based on the typical waveform templates Π(Φ,t) described above, the therapy parameter determination algorithm 4329 increases the treatment pressure Pt to P₀+A (known as the IPAP) at the start of, or during, or inspiration and decreases the treatment pressure Pt to the base pressure P₀ (known as the EPAP) at the start of, or during, expiration.

In some forms of bi-level therapy, the IPAP is a treatment pressure that has the same purpose as the treatment pressure in CPAP therapy modes, and the EPAP is the IPAP minus the amplitude A, which has a “small” value (a few cmH2O) sometimes referred to as the Expiratory Pressure Relief (EPR). Such forms are sometimes referred to as CPAP therapy with EPR, which is generally thought to be more comfortable than straight CPAP therapy. In CPAP therapy with EPR, either or both of the IPAP and the EPAP may be constant values that are hard-coded or manually entered to the RPT device 4000. Alternatively, the therapy parameter determination algorithm 4329 may repeatedly compute the IPAP and/or the EPAP during CPAP with EPR. In this alternative, the therapy parameter determination algorithm 4329 repeatedly computes the EPAP and/or the IPAP as a function of indices or measures of sleep disordered breathing returned by the respective algorithms in the therapy engine module 4320 in analogous fashion to the computation of the base pressure P₀ in APAP therapy described above.

In other forms of bi-level therapy, the amplitude A is large enough that the RPT device 4000 does some or all of the work of breathing of the patient 1000. In such forms, known as pressure support ventilation therapy, the amplitude A is referred to as the pressure support, or swing. In pressure support ventilation therapy, the IPAP is the base pressure P₀ plus the pressure support A, and the EPAP is the base pressure P₀.

In some forms of pressure support ventilation therapy, known as fixed pressure support ventilation therapy, the pressure support A is fixed at a predetermined value, e.g. 10 cmH2O. The predetermined pressure support value is a setting of the RPT device 4000, and may be set for example by hard-coding during configuration of the RPT device 4000 or by manual entry through the input device 4220.

In other forms of pressure support ventilation therapy, broadly known as servo-ventilation, the therapy parameter determination algorithm 4329 takes as input some currently measured or estimated parameter of the respiratory cycle (e.g. the current measure Vent of ventilation) and a target value of that respiratory parameter (e.g. a target value Vtgt of ventilation) and repeatedly adjusts the parameters of equation (1) to bring the current measure of the respiratory parameter towards the target value. In a form of servo-ventilation known as adaptive servo-ventilation (ASV), which has been used to treat CSR, the respiratory parameter is ventilation, and the target ventilation value Vtgt is computed by the target ventilation determination algorithm 4328 from the typical recent ventilation Vtyp, as described above.

In some forms of servo-ventilation, the therapy parameter determination algorithm 4329 applies a control methodology to repeatedly compute the pressure support A so as to bring the current measure of the respiratory parameter towards the target value. One such control methodology is Proportional-Integral (PI) control. In one implementation of PI control, suitable for ASV modes in which a target ventilation Vtgt is set to slightly less than the typical recent ventilation Vtyp, the pressure support A is repeatedly computed as:

$$A = G \int ( Vent - Vtgt ) dt \qquad ( 2 )$$

where G is the gain of the PI control. Larger values of gain G can result in positive feedback in the therapy engine module 4320. Smaller values of gain G may permit some residual untreated CSR or central sleep apnea. In some implementations, the gain G is fixed at a predetermined value, such as −0.4 cmH2O/(L/min)/sec. Alternatively, the gain G may be varied between therapy sessions, starting small and increasing from session to session until a value that substantially eliminates CSR is reached. Conventional means for retrospectively analysing the parameters of a therapy session to assess the severity of CSR during the therapy session may be employed in such implementations. In yet other implementations, the gain G may vary depending on the difference between the current measure Vent of ventilation and the target ventilation Vtgt.

Other servo-ventilation control methodologies that may be applied by the therapy parameter determination algorithm 4329 include proportional (P), proportional-differential (PD), and proportional-integral-differential (PID).

The value of the pressure support A computed via equation (2) may be clipped to a range defined as [Amin, Amax]. In this implementation, the pressure support A sits by default at the minimum pressure support Amin until the measure of current ventilation Vent falls below the target ventilation Vtgt, at which point A starts increasing, only falling back to Amin when Vent exceeds Vtgt once again.

The pressure support limits Amin and Amax are settings of the RPT device 4000, set for example by hard-coding during configuration of the RPT device 4000 or by manual entry through the input device 4220.

In pressure support ventilation therapy modes, the EPAP is the base pressure P₀. As with the base pressure P₀ in CPAP therapy, the EPAP may be a constant value that is prescribed or determined during titration. Such a constant EPAP may be set for example by hard-coding during configuration of the RPT device 4000 or by manual entry through the input device 4220. This alternative is sometimes referred to as fixed-EPAP pressure support ventilation therapy. Titration of the EPAP for a given patient may be performed by a clinician during a titration session with the aid of PSG, with the aim of preventing obstructive apneas, thereby maintaining an open airway for the pressure support ventilation therapy, in similar fashion to titration of the base pressure P₀ in constant CPAP therapy.

Alternatively, the therapy parameter determination algorithm 4329 may repeatedly compute the base pressure P₀ during pressure support ventilation therapy. In such implementations, the therapy parameter determination algorithm 4329 repeatedly computes the EPAP as a function of indices or measures of sleep disordered breathing returned by the respective algorithms in the therapy engine module 4320, such as one or more of flow limitation, apnea, hypopnea, patency, and snore. Because the continuous computation of the EPAP resembles the manual adjustment of the EPAP by a clinician during titration of the EPAP, this process is also sometimes referred to as auto-titration of the EPAP, and the therapy mode is known as auto-titrating EPAP pressure support ventilation therapy, or auto-EPAP pressure support ventilation therapy.

### 5.10.2 High Flow Therapy

In other forms of respiratory therapy, the pressure of the flow of air is not controlled as it is for respiratory pressure therapy. Rather, the central controller 4230 controls the pressure generator 4140 to deliver a flow of air whose device flow rate Qd is controlled to a treatment or target flow rate Qtgt that is typically positive throughout the patient's breathing cycle. Such forms are generally grouped under the heading of flow therapy. In flow therapy, the treatment flow rate Qtgt may be a constant value that is hard-coded or manually entered to the RPT device 4000. If the treatment flow rate Qtgt is sufficient to exceed the patient's peak inspiratory flow rate, the therapy is generally referred to as high flow therapy (HFT). Alternatively, the treatment flow rate may be a profile Qtgt(t) that varies over the respiratory cycle.

### 5.11 GLOSSARY

For the purposes of the present technology disclosure, in certain forms of the present technology, one or more of the following definitions may apply. In other forms of the present technology, alternative definitions may apply.

### 5.11.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H2O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH2O, g-f/cm2 and hectopascal. 1 cmH2O is equal to 1 g-f/cm2 and is approximately 0.98 hectopascal (1 hectopascal=100 Pa=100 N/m2=1 millibar˜0.001 atm). In this specification, unless otherwise stated, pressure is given in units of cmH2O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.11.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.11.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 5.11.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr):

These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Ti): The duration of the inspiratory portion of the respiratory flow rate waveform.

Exhalation Time (Te): The duration of the expiratory portion of the respiratory flow rate waveform.

Total Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.11.3 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts, pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator, or other respiratory therapy device such as an RPT device or portable oxygen concentrator, delivers a volume of breathable gas to a spontaneously breathing patient, it is said to be triggered to do so. Triggering usually takes place at or near the initiation of the respiratory portion of the breathing cycle by the patient's efforts.

### 5.11.4 Anatomy

### 5.11.4.1 Anatomy of the Face

Ala: the external outer wall or “wing” of each nostril (plural: alar)

### Alar Angle:

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius):

Lip, upper (labrale superius):

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.11.4.2 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.11.5 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.11.6 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 5.11.6.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 5.11.6.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical-topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 5.11.6.3 Space Curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 3O and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S

With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 5.11.6.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 5.12 OTHER REMARKS

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.13 REFERENCE SIGNS LIST

| | |
|---|---|
| patient | 1000 |
| bed partner | 1100 |
| patient interface | 3000 |
| seal forming structure | 3100 |
| plenum chamber | 3200 |
| chord | 3210 |
| superior point | 3220 |
| inferior point | 3230 |
| positioning and stabilising structure | 3300 |
| vent | 3400 |
| connection port | 3600 |
| forehead support | 3700 |
| rpt device | 4000 |
| external housing | 4010 |
| upper portion | 4012 |
| lower portion | 4014 |
| panel | 4015 |
| chassis | 4016 |
| handle | 4018 |
| pneumatic block | 4020 |
| air filter | 4110 |
| inlet air filter | 4112 |
| muffler | 4120 |
| outlet muffler | 4124 |
| pressure generator | 4140 |
| blower | 4142 |
| motor | 4144 |
| anti-spillback valve | 4160 |
| air circuit | 4170 |
| tube | 4175 |
| supplementary gas | 4180 |
| electrical components | 4200 |
| pcba | 4202 |
| electrical power supply | 4210 |
| input device | 4220 |
| transducers | 4270 |
| humidifier | 5000 |
| humidifier inlet | 5002 |
| humidifier outlet | 5004 |
| humidifier base | 5006 |
| humidifier reservoir | 5110 |
| conductive portion | 5120 |
| reservoir dock | 5130 |
| locking lever | 5135 |
| water level indicator | 5150 |
| heating element | 5240 |
| monitoring apparatus | 7100 |
| monitoring device | 7200 |
| processor | 7210 |
| storage medium | 7230 |
| stored data | 7240 |
| processor control instructions | 7250 |
| data input interface | 7260 |
| sensing kit | 8000 |
| patient interface | 8010 |
| plenum chamber | 8012 |
| seal-forming structure | 8014 |
| frame | 8016 |
| positioning and stabilizing structure | 8020 |
| first strap | 8022 |
| second strap | 8024 |
| first circuit board assembly | 8030 |
| first circuit board | 8032 |
| battery | 8034 |
| USB communication module | 8036 |
| wires | 8038 |
| housing | 8040 |
| first piece | 8042 |
| second piece | 8044 |
| flat surface | 8046 |
| rim | 8048 |
| strap | 8050 |
| flat portion | 8052 |
| straps | 8054 |
| retaining features | 8056 |
| notch | 8058 |
| frame | 8060 |
| opening | 8062 |
| securing strap | 8064 |
| openings | 8066 |
| docking station | 8070 |
| docking board | 8072 |
| housing | 8074 |
| pogo pins | 8076 |
| cradle | 8078 |
| recessed portion | 8080 |
| openings | 8082 |
| end portion | 8084 |
| base | 8086 |
| slot | 8088 |
| clamp | 8090 |
| leg | 8092 |
| crossbar | 8094 |
| protrusion | 8096 |
| slot | 8098 |
| second circuit board assembly | 9000 |
| second circuit board | 9010 |
| pressure sensor | 9012 |
| CO₂ sensor | 9014 |
| temperature and humidity sensor | 9016 |
| grommet | 9020 |
| first flange | 9022 |
| second flange | 9024 |
| lumen | 9026 |
| protrusion | 9028 |
| gasket | 9030 |
| sensor | 9032 |
| grommet | 9040 |
| first flange | 9042 |
| second flange | 9044 |
| rim | 9046 |
| connector | 9050 |
| patient interface | 10000 |
| plenum chamber | 10012 |
| seal-forming structure | 10014 |
| frame | 10016 |
| vent | 10018 |
| second circuit board assembly | 10020 |
| second circuit board | 10022 |
| cable | 10024 |
| patient interface | 11000 |
| plenum chamber | 11012 |
| seal-forming structure | 11014 |
| frame | 11016 |
| vent | 11018 |
| second circuit board assembly | 11020 |
| second circuit board | 11022 |
| adhesive | 11024 |
| sensor | 11026 |
| opening | 11028 |
| opening | 11030 |
| barb | 11032 |
| inner surface | 11034 |
| external surface | 11036 |
| reporting system | 12000 |
| external device | 12002 |
| user interface | 12010 |
| screen | 12020 |
| pane | 12022 |
| pane | 12024 |
| screen | 12030 |
| plot | 12032 |
| plot | 12034 |
| plot | 12036 |
| plot | 12038 |
| screen | 12040 |
| selection pane | 12042 |
| selection interface | 12044 |
| details pane | 12046 |
| graph | 12048 |
| screen | 12050 |
| graph | 12052 |
| key | 12053 |
| graph | 12054 |
| key | 12055 |
| graph | 12056 |
| key | 12057 |
| screen | 12060 |
| graph | 12062 |
| key | 12063 |
| graph | 12064 |
| key | 12065 |
| plot | 12066 |
| plot | 12068 |
| screen | 12070 |
| graph | 12072 |
| key | 12074 |
| screen | 12080 |
| graph | 12082 |
| key | 12083 |
| selection pane | 12084 |
| detail pane | 12086 |
| system | 13000 |
| bus | 13010 |
| CPU | 13020 |
| ROM | 13030 |
| RAM | 13040 |
| input and output ports | 13050 |

1. A system for sensing parameters associated with a respiratory therapy (“RPT”) system, the system comprising:
a first housing that includes a first circuit board, wherein the first circuit board includes at least one control element;
a second housing, which is separate from the first housing, that is removably attachable to/from a patient interface of the RPT system, the second housing including a second circuit board with a plurality of sensors disposed thereon, wherein at least one of the plurality of sensors is configured to sense, when the second housing is attached to the patient interface, at least one physical phenomena within a plenum chamber of the patient interface; and
a connector configured to electrically connect the first circuit board to the second circuit board.

2. The system of claim 1, wherein another one of the plurality of sensors is configured to sense, when the second housing is attached to the patient interface, at least one physical phenomena an atmosphere outside of the plenum chamber.

3. The system of claim 1, wherein the plurality of sensors include at least two of a pressure sensor, a humidity sensor, a temperature sensor, and a CO₂ sensor.

4. The system of claim 1, wherein a grommet houses the second circuit board.

5. The system of claim 4, wherein a portion of the patient interface defining the plenum chamber includes an opening in communication with the plenum chamber and accommodating the grommet.

6. The system of claim 5, wherein the grommet forms a seal with the plenum chamber when disposed in the opening.

7. The system of claim 5, wherein the grommet defines a lumen.

8. The system of claim 7, wherein, in a configuration in which the grommet is disposed within the opening, the lumen is in communication with an atmosphere outside of the plenum chamber.

9. The system of claim 7, wherein at least one of the plurality of sensors is at least partially disposed within the lumen.

10. The system of claim 1, wherein the first housing is configured to be attached to a strap of the patient interface.

11. The system of claim 1, wherein, when the second housing is coupled to the patient interface, the first housing is located outside of the plenum chamber.

12. The system of claim 1, further including a docking station for removably receiving the first circuit board.

13. The system of claim 12, wherein the docking station includes circuitry for charging a battery of the first circuit board.

14. The system of claim 12, wherein the docking station includes circuitry for communicating with an external device.

15. The system of claim 14, wherein the circuitry for communicating with an external device includes a USB module.

16. The system of claim 1, wherein at least one aperture is defined in the first housing.

17. The system of claim 16, wherein the aperture in the first housing is configured to receive a pin that electrically contacts the first circuit board.

18. The system of claim 1, wherein the connector includes an I2C bus.

19. The system of claim 1, wherein the connector is removably connectable to a third circuit board contained in a third housing, the third circuit board having disposed thereon a second plurality of sensors, wherein the second plurality of sensors is a different arrangement of sensors from the plurality of sensors disposed with the second circuit board.

20. The system of claim 1, wherein at least one of the first circuit board or the second circuit board includes a wireless transceiver.

21. The system of claim 1,
wherein at least one sensor of the plurality of sensors is mounted on a circuit board, wherein the circuit board is configured to be coupled to a patient interface of the RPT system, such that the at least one sensor is configured to sense a parameter within a plenum chamber of the patient interface and a parameter of an atmosphere outside of the plenum chamber.

22. The system of claim 21, wherein a portion of the patient interface defining the plenum chamber includes an opening in communication with the plenum chamber, wherein the circuit board is coupled to a grommet, and wherein the opening is configured to accommodate the grommet.

23. The system of claim 21, wherein the circuit board is configured to be mounted within the plenum chamber, and wherein the sensor is configured to sense the parameter of the atmosphere via an opening of a vent in fluid communication with the plenum chamber.

24. The system of claim 4,
a sensor mounted to the second circuit board, wherein a portion of the patient interface defining a plenum chamber includes an opening in communication with the plenum chamber and accommodating the grommet.

25. The system of claim 1, further comprising:
the respiratory the respiratory therapy (“RPT”) system for providing a positive pressure flow of gas to an airway of a patient, the RPT system including:
a patient interface configured to interface with at least one airway of the patient, and
respiratory equipment configured to supply the positive pressure flow at flow parameters to the patient interface; and
wherein at least one of the first and second circuit boards includes at least one sensor, the at least one of the first and second circuit boards coupled to the patient interface such that the at least one sensor is configured to sense a parameter within a plenum chamber of the patient interface,
wherein at least one of the flow parameters is adjusted based on the parameter sensed by the sensor.

26. The system of claim 1, wherein the first circuit board includes an accelerometer.
